(19) United States
(12) Patent Application Publication
(10) Pub. No.: US 2022/0170025 A1
(43) Pub. Date: Jun. 2, 2022

(54) COMPOSITIONS AND METHODS FOR INHIBITING GENE EXPRESSION IN THE CENTRAL NERVOUS SYSTEM

(71) Applicant: Dicerna Pharmaceuticals Inc., Lexington, MA (US)

(72) Inventors: Bob Dale BROWN, Littleton, MA (US); Maire OSBORN, Lincoln, MA (US); Weimin WANG, Waltham, MA (US)

(21) Appl. No.: 17/594,094

(22) PCT Filed: Apr. 3, 2020
(86) PCT No.: PCT/US2020/026717

§ 371 Date: Oct. 1, 2021

Related U.S. Application Data
(60) Provisional application No. 62/829,595, filed on Apr. 4, 2019.

(51) Int. Cl. C12N 15/113

(52) U.S. Cl. CPC C12N 15/1137; C12N 2310/14; C12N 2310/531; C12N 2310/321; C12N 2310/352; C12N 2310/351; C12N 2320/32; C12N 2310/33; C12N 2310/315; C12N 2310/322

(57) ABSTRACT

This disclosure relates to the use of RNA oligonucleotides, compositions and methods useful for reducing ALDH2 or other target gene expression, in the central nervous system. In some embodiments, the oligonucleotide is used in methods of treating neurological diseases. Stable oligonucleotide derivatives that have enhanced activity in the central nervous system are provided.

### CROSS-REFERENCE TO RELATED APPLICATIONS

This application claims the benefit under 35 U.S.C. § 119(e) of U.S. Provisional Application No. 62/829,595, filed Apr. 4, 2019, the entire contents of which are incorporated herein by reference.

### FIELD OF THE INVENTION

The present application relates to the use of RNA interference oligonucleotides for the degradation of specific target mRNA's, particularly uses relating to the treatment of neurological conditions.

### REFERENCE TO THE SEQUENCE LISTING

The present application is being filed along with a Sequence Listing in electronic format. The Sequence Listing is provided as a file entitled 400930-021WO_ST25.txt created on Apr. 3, 2020 and is 128 kilobytes in size. The information in electronic format of the Sequence Listing is incorporated herein by reference in its entirety.

### BACKGROUND OF THE INVENTION

RNA interference (RNAi) is an innate cellular process that involves multiple RNA-protein interactions. Its gene silencing activity is activated when a double-stranded RNA (dsRNA) molecule of greater than 19 duplex nucleotides enters the cells, causing degradation of both the dsRNA and single stranded RNA (endogenous mRNA) of identical sequences.

More specifically, the RNA interference (RNAi) mechanism inhibits or activates gene expression at the stage of translation or by hindering the transcription of specific genes. RNAi targets include RNA from viruses and transposons, and RNAi inhibition of expression also plays a role in regulating development and genome maintenance. The RNAi pathway is initiated by the enzyme dicer, which cleaves long, double-stranded RNA (dsRNA) molecules into short fragments of 20-25 base pairs. One of the two strands of each fragment, known as the guide strand, is then incorporated into the RNA-induced silencing complex (RISC). The RISC is a multiprotein complex, specifically a ribonucleoprotein, which incorporates one strand of a single-stranded RNA the “antisense strand” or “guide strand” (ssRNA) fragment to guide RISC to a complementary mRNA for subsequent endonucleolytic cleavage. Once found, one of the proteins in RISC, called Argonaute, activates and cleaves the mRNA.

In general, difficulties in the use of RNAi technology in the past have included off-target effects related to the use of guide strands insufficiently tailored to affect specific genes, delivery to multiple organ systems where gene expression of the target gene may be desirable and having the capability to target oligonucleotides to organ systems other than the liver where the characteristics of hepatocytes assist in the uptake and effectiveness of RNAi technology.

In terms of pathologies of the Central Nervous System (“CNS”) most pharmacotherapies currently being used for treatment of neurodegenerative or inflammatory CNS disorders target molecules that are localized downstream in the pathogenic cascade. Therefore, their effects are often not specific and are moderate or simply ineffective with regard to disease modulation. Other approaches that may add to the medical arsenal are those that focus on different methods of modulating or controlling a disease. Among these innovative therapeutic strategies is the ‘silencing’ of genes that cause or directly contribute to disease phenotypes using RNAi technologies. The difficulties in using this therapeutic avenue have been identifying specific candidate genes, specific targeting to the CNS, durability of therapeutic effect and the exit from the CNS of RNAi modalities that could affect other tissues.

The aldehyde dehydrogenase-2 (ALDH2) gene encodes an important biologically active enzyme, ALDH2. ALDH2 participates in the metabolism and detoxification of aldehyde and metabolizes short-chain aliphatic aldehydes and converted acetaldehyde into acetate it is active in the human liver. ALDH2 has been shown involved in the metabolism of other biogenic aldehydes, such as 4-hydroxynonenal, 3,4-dihydroxyphenylacetaldehyde, and 3,4-dihydroxyphenylglycoaldehyde. Recent studies have indicated that ALDH2 is also expressed in the CNS where it exerts protective effects on the cardio-cerebral vascular system and central nervous system. Single nucleotide polymorphisms (SNPs) of the ALDH2 gene have been reported to be associated with the risks for several neurological diseases, such as neurodegenerative diseases, cognitive disorders, and anxiety disorders. Removing or inhibiting the ALDH2 gene in the CNS prevents or limits the biological activity of the active enzyme and is relatively easily measured.

### BRIEF SUMMARY OF THE INVENTION

Aspects of the disclosure relate to oligonucleotides and related methods for treating a neurological disease in a subject. In some embodiments, potent RNAi oligonucleotides are provided for their selective activity in the CNS. In the present invention the oligonucleotides administered into the CNS are effective at delivering an ALDH2 targeting guide strand that loads into the RISC complex and that thereafter is effective in the inhibition of ALDH2 expression in the central nervous system of a subject via the cleavage of ALDH2 mRNAs. In some embodiments, RNAi oligonucleotides provided herein target key regions of ALDH2 mRNA (referred to as hotspots) that are particularly amenable to targeting using such oligonucleotide-based approaches (see Table 5). In some embodiments, RNAi oligonucleotides provided herein incorporate modified phosphates, nicked tetraloop structures, and/or other modifications that improve activity, bioavailability and/or minimize the extent of enzymatic degradation after in vivo administration to the central nervous system. The ALDH2 gene targeting sequence, according to the present invention, could be replaced with a guide strand directed to a gene sequence of interest in a fashion that would allow the specific degradation of mRNA in the CNS and thereby degrade or inhibit the production of a protein of interest. Where this protein is a contributor to gain of function pathology—the negative aspects of the pathology are reduced or eliminated while the RISC complex remains active in cleaving the target mRNA. Other oligonucleotides of the current invention can also be put into to the CNS to modulate or inhibit the expression of specific target genes in a therapeutically meaningful way.

Some aspects of the present disclosure provide methods of reducing expression of ALDH2 in a subject, the method comprising administering to the cerebrospinal fluid of the subject an oligonucleotide comprising an antisense strand of 15 to 30 nucleotides in length, wherein the antisense strand has a region of complementarity to a target sequence of ALDH2 as set forth in any one of SEQ ID NOs: 601-607, wherein the region of complementarity is at least 12 contiguous nucleotides in length. In some embodiments, the region of complementarity is fully complementary to the target sequence of ALDH2. In some embodiments, the antisense strand is 19 to 27 nucleotides in length.

In some embodiments, the oligonucleotide further comprises a sense strand of 15 to 40 nucleotides in length, wherein the sense strand forms a duplex region with the antisense strand. In some embodiments, the sense strand is 19 to 40 nucleotides in length.

In some embodiments, the duplex region is at least 12 nucleotides in length. In some embodiments, the region of complementarity to ALDH2 is at least 13 contiguous nucleotides in length.

In some embodiments, the antisense strand comprises a sequence as set forth in any one of SEQ ID NOs: 591-600. In some embodiments, the sense strand comprises a sequence as set forth in any one of SEQ ID NOs: 581-590, 608, and 609. In some embodiments, the sense strand consists of a sequence as set forth in any one of SEQ ID NOs: 591-600. In some embodiments, the antisense strand consists of a sequence as set forth in any one of SEQ ID NOs: 581-590, 608, and 609.

In some embodiments, the oligonucleotide comprises at least one modified nucleotide. In some embodiments, the modified nucleotide comprises a 2′-modification. In some embodiments, the 2′-modification is a modification selected from: 2′-aminoethyl, 2′-fluoro, 2′-O-methyl, 2′-O-methoxyethyl, 2′-aminodiethoxymethanol, 2′-adem, and 2′-deoxy-2′-fluoro-β-d-arabinonucleic acid. In some embodiments, all of the nucleotides of the oligonucleotide are modified.

In some embodiments, the oligonucleotide comprises at least one modified internucleotide linkage. In some embodiments, the at least one modified internucleotide linkage is a phosphorothioate linkage.

In some embodiments, the oligonucleotide comprises a phosphorothioate linkage between one or more of: positions 1 and 2 of the sense strand, positions 1 and 2 of the antisense strand, positions 2 and 3 of the antisense strand, positions 3 and 4 of the antisense strand, positions 20 and 21 of the antisense strand, and/or positions 21 and 22 of the antisense strand. In some embodiments, the oligonucleotide has a phosphorothioate linkage between each of: positions 1 and 2 of the sense strand, positions 1 and 2 of the antisense strand, positions 2 and 3 of the antisense strand, positions 20 and 21 of the antisense strand, and positions 21 and 22 of the antisense strand.

In some embodiments, the 4′-carbon of the sugar of the 5′-nucleotide of the antisense strand comprises a phosphate analog. In some embodiments, the phosphate analog is oxymethylphosphonate, vinylphosphonate, or malonylphosphonate.

In some embodiments, a uridine present at the first position of an antisense strand comprises a phosphate analog. In some embodiments, the oligonucleotide comprises the following structure at position 1 of the antisense strand:

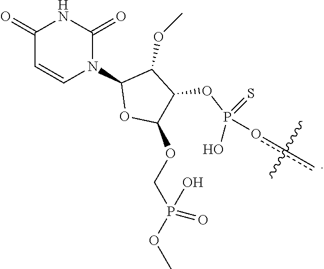

In some embodiments, the sense strand comprises at its 3′-end a stem-loop set forth as: S₁-L-S₂, wherein S₁ is complementary to S₂, and wherein L forms a loop between S₁ and S₂ of 3 to 5 nucleotides in length. In some embodiments, L is a tetraloop. In some embodiments, L is 4 nucleotides in length. In some embodiments, L comprises a sequence set forth as GAAA.

In some embodiments, one or more of the nucleotides of the GAAA sequence at positions 27-30 on the sense strand is conjugated to a monovalent GalNAc moiety. In some embodiments, each of the nucleotides of the GAAA sequence at positions 27-30 on the sense strand is conjugated to a monovalent GalNAc moiety. In some embodiments, each of A of the GAAA sequence (at positions 28-30) on the sense strand is conjugated to a monovalent GalNAc moiety. In some embodiments, an oligonucleotide herein comprises a monovalent GalNAc attached to a Guanidine nucleotide, referred to as [ademG-GalNAc] or 2′-aminodiethoxymethanol-Guanidine-GalNAc, as depicted below:

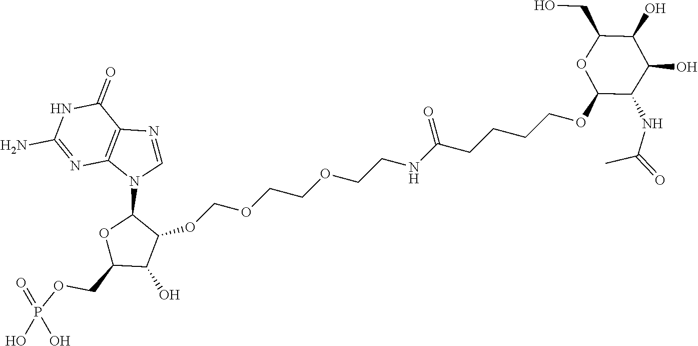 [SMILES: CC(=O)N[C@@H]1[C@@H](OCCCCC(=O)NCCOCCOCO[C@@H]2[C@H](O)[C@@H](COP(=O)(O)O)O[C@H]2n2cnc3c(=O)[nH]c(N)nc32)O[C@@H](CO)[C@@H](O)[C@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below.

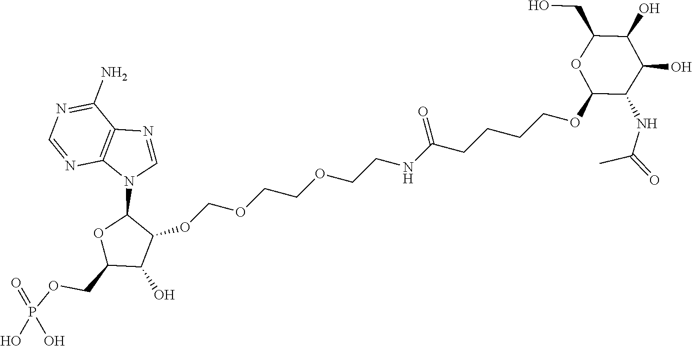 [SMILES: CC(=O)N[C@@H]1[C@@H](OCCCCC(=O)NCCOCCOCO[C@@H]2[C@H](O)[C@@H](COP(=O)(O)O)O[C@H]2n2cnc3c(N)ncnc32)O[C@@H](CO)[C@@H](O)[C@H]1O]

In some embodiments, the GAAA motif at positions 27-30 on the sense strand comprises the structure:

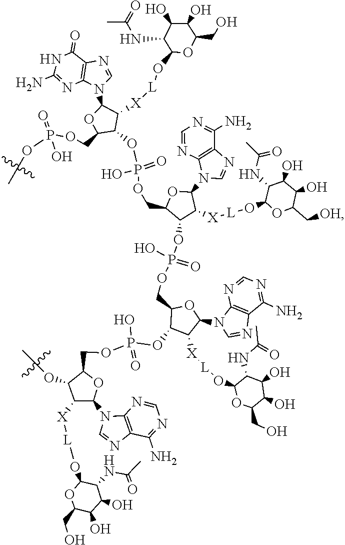

wherein:

L represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and X is O, S, or N.

In some embodiments, L is an acetal linker. In some embodiments, X is O.

In some embodiments, the GAAA sequence at positions 27-30 on the sense strand comprises the structure:

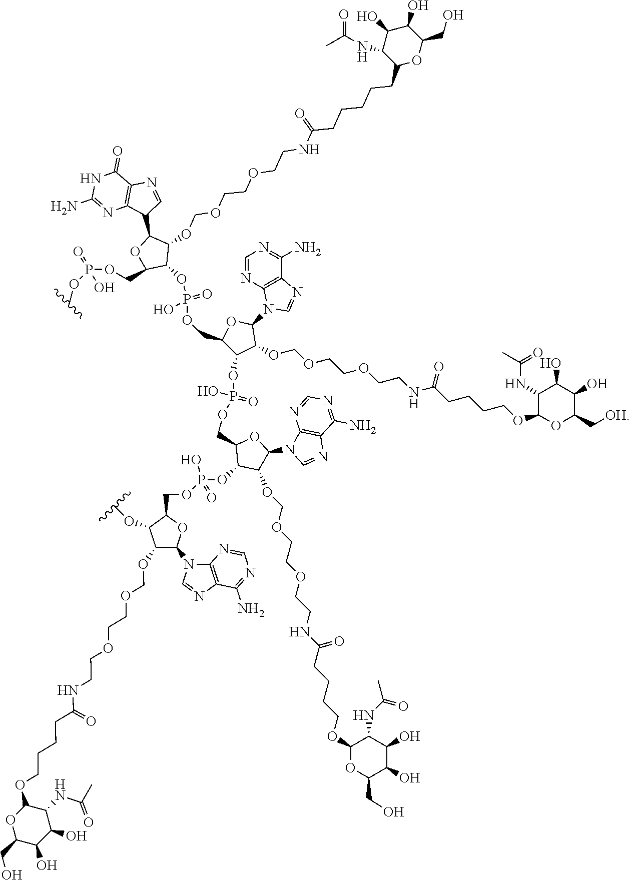

In some embodiments, each of the A in the GAAA sequence is conjugated to a GalNAc moiety (e.g., at positions 28-30 on the sense strand). In some embodiments, the GalNAc moiety conjugated to each of A has the structure illustrated above, except that G is unmodified or has a 2′ modification on the sugar moiety. In some embodiments, the G in the GAAA sequence comprises a 2′-O-methyl modification (e.g., 2′-O-methyl or 2′-O-methoxyethyl), and each of A in the GAAA sequence is conjugated to a GalNAc moiety, such as in portions of the structures illustrated above.

In some embodiments, the G in the GAAA sequence comprises a 2′-OH. In some embodiments, each of the nucleotides in the GAAA sequence comprises a 2′-O-methyl modification. In some embodiments, each of the A in the GAAA sequence comprises a 2′-OH and the G in the GAAA sequence comprises a 2′-O-methyl modification. In some embodiments, each of the A in the GAAA sequence comprises a 2′-O-methoxyethyl modification and the G in the GAAA sequence comprises a 2′-O-methyl modification. In some embodiments, each of the A in the GAAA sequence comprises a 2′-adem modification and the G in the GAAA sequence comprises a 2′-O-methyl modification.

In some embodiments, the antisense strand and the sense strand are not covalently linked.

In some embodiments, the oligonucleotide is administered intrathecally, intraventricularly, intracavitary, or interstitially. In some embodiments, the oligonucleotide is administered via injection or infusion.

In some embodiments, the subject has a neurological disorder. In some embodiments, the neurological disorder is selected from: neurodegenerative diseases, cognitive disorders, and anxiety disorders.

In some embodiments, the method of reducing expression of ALDH2 in a subject comprises administering to the cerebrospinal fluid of the subject an oligonucleotide comprising an antisense strand and a sense strand,

wherein the antisense strand is 21 to 27 nucleotides in length and has a region of complementarity to ALDH2,

wherein the sense strand comprises at its 3′-end a stem-loop set forth as: S₁-L-S₂, wherein S₁ is complementary to S₂, and wherein L forms a loop between S₁ and S₂ of 3 to 5 nucleotides in length,

and wherein the antisense strand and the sense strand form a duplex structure of at least 12 nucleotides in length but are not covalently linked.

In some embodiments, the method of reducing expression of ALDH2 in a subject comprises administering to the cerebrospinal fluid of the subject an oligonucleotide comprising an antisense strand and a sense strand that are not covalently linked,

wherein the antisense strand comprises a sequence as set forth in SEQ ID NO: 595 and the sense strand comprises a sequence as set forth in SEQ ID NO: 585,

wherein the sense strand comprises at its 3′-end a stem-loop set forth as: S₁-L-S₂, wherein S₁ is complementary to S₂, and wherein L is a tetraloop comprising a sequence set forth as GAAA, and wherein the GAAA sequence comprises a structure selected from the group consisting of:

(i) each of the A in GAAA sequence is conjugated to a GalNAc moiety, and the G in the GAAA sequence comprises a 2′-O-methyl modification;

(ii) each of the A in GAAA sequence is conjugated to a GalNAc moiety, and the G in the GAAA sequence comprises a 2′-OH;

(iii) each of the nucleotide in the GAAA sequence comprises a 2′-O-methyl modification;

(iv) each of the A in the GAAA sequence comprises a 2′-OH and the G in the GAAA sequence comprises a 2′-O-methyl modification;

(v) each of the A in the GAAA sequence comprises a 2′-O-methoxyethyl modification and the G in the GAAA sequence comprises a 2′-O-methyl modification; and

(vi) each of the A in the GAAA sequence comprises a 2′-aminodiethoxymethanol modification and the G in the GAAA sequence comprises a 2′-O-methyl modification.

In some embodiments, the method of reducing expression of ALDH2 in a subject comprises administering to the cerebrospinal fluid of the subject an oligonucleotide comprising an antisense strand and a sense strand that are not covalently linked, wherein the antisense strand comprises a sequence as set forth in SEQ ID NO: 595 and the sense strand comprises a sequence as set forth in SEQ ID NO: 609.

In some embodiments, the oligonucleotide reduces expression detectable in somatosensory cortex, hippocampus, frontal cortex, striatum, hypothalamus, cerebellum, and/or spinal cord.

Other aspects of the present disclosure provide methods of reducing expression of a gene of interest in a subject, the method comprising administering to the cerebrospinal fluid of the subject an oligonucleotide comprising an antisense strand of 15 to 30 nucleotides in length, wherein the antisense strand has a region of complementarity to a target sequence of said gene of interest that expresses in the CNS, wherein the region of complementarity is at least 12 contiguous nucleotides in length.

In some embodiments, the gene of interest is selected from the group consisting of ALDH2, Ataxin-1, Ataxin-3, APP, BACE1, DYT1, and SOD1.

In some embodiments, the oligonucleotide reduces expression detectable in somatosensory cortex, hippocampus, frontal cortex, striatum, hypothalamus, cerebellum, and/or spinal cord.

In some embodiments, the oligonucleotide further comprising elements that are degraded by nucleases outside the CNS such that said nucleotide is no longer capable of reducing expression of a gene of interest in a subject in tissues outside the CNS.

In some embodiments, the oligonucleotide further comprises modifications such that it cannot easily exit the CNS.

Other aspects of the present disclosure provide methods of treating a neurological disorder, the method comprising administering to the cerebrospinal fluid of a subject in need thereof an oligonucleotide comprising an antisense strand of 15 to 30 nucleotides in length, wherein the antisense strand has a region of complementarity to a target sequence of ALDH2 as set forth in any one of SEQ ID NOs: 601-607, wherein the region of complementarity is at least 12 contiguous nucleotides in length.

In some embodiments, the method comprises administering to the cerebrospinal fluid of a subject in need thereof an oligonucleotide comprising an antisense strand and a sense strand,

wherein the antisense strand is 21 to 27 nucleotides in length and has a region of complementarity to ALDH2,

wherein the sense strand comprises at its 3′-end a stem-loop set forth as: S₁-L-S₂, wherein S₁ is complementary to S₂, and wherein L forms a loop between S₁ and S₂ of 3 to 5 nucleotides in length,

and wherein the antisense strand and the sense strand form a duplex structure of at least 12 nucleotides in length but are not covalently linked.

In some embodiments, the neurological disorder is a neurodegenerative disease. In some embodiments, the neurological disorder is an anxiety disorder.

In some embodiments, the oligonucleotide is administered intrathecally, intraventricularly, intracavitary, or interstitially. In some embodiments, the oligonucleotide is administered via injection or infusion.

In some embodiments, the oligonucleotide reduces expression detectable in somatosensory cortex, hippocampus, frontal cortex, striatum, hypothalamus, cerebellum, and/or spinal cord.

Other aspects of the present disclosure provide oligonucleotides comprising an antisense strand and a sense strand,

wherein the antisense strand is 21 to 27 nucleotides in length and has a region of complementarity to ALDH2,

wherein the sense strand comprises at its 3′-end a stem-loop set forth as: S₁-L-S₂, wherein S₁ is complementary to S₂, and wherein L is a tetraloop and comprises a sequence set forth as GAAA, wherein the GAAA sequence comprises a structure selected from the group consisting of:

(i) each of the A in GAAA sequence is conjugated to a GalNAc moiety, and the G in the GAAA sequence comprises a 2′-O-methyl modification;

(ii) each of the A in GAAA sequence is conjugated to a GalNAc moiety, and the G in the GAAA sequence comprises a 2′-OH;

(iii) each of the nucleotide in the GAAA sequence comprises a 2′-O-methyl modification;

(iv) each of the A in the GAAA sequence comprises a 2′-OH and the G in the GAAA sequence comprises a 2′-O-methyl modification;

(v) each of the A in the GAAA sequence comprises a 2′-O-methoxyethyl modification and the G in the GAAA sequence comprises a 2′-O-methyl modification; and

(vi) each of the A in the GAAA sequence comprises a 2′-adem modification and the G in the GAAA sequence comprises a 2′-O-methyl modification,

and wherein the antisense strand and the sense strand form a duplex structure of at least 12 nucleotides in length but are not covalently linked.

In some embodiments, the antisense strand comprises a sequence set forth in any one of SEQ ID NOs: 591-600. In some embodiments, the sense strand comprises a sequence set forth in any one of SEQ ID NOs: 581-590. Compositions comprising these oligonucleotides and an excipient are provided. In some embodiments, a method of reducing expression ALDH2 in a subject comprises administering the composition to the cerebrospinal fluid of the subject. In some embodiments, a method of treating a neurological disease in a subject in need thereof comprises administering the composition to the cerebrospinal fluid of the subject.

Other aspects of the present disclosure provide methods of reducing expression of a target gene in a subject, the method comprising administering an oligonucleotide to the cerebrospinal fluid of the subject, wherein the oligonucleotide comprises an antisense strand and a sense strand,

wherein the antisense strand is 21 to 27 nucleotides in length and has a region of complementarity to the target gene,

wherein the sense strand comprises at its 3′-end a stem-loop set forth as: S₁-L-S₂, wherein S₁ is complementary to S₂, and wherein L forms a loop between S₁ and S₂ of 3 to 5 nucleotides in length,

and wherein the antisense strand and the sense strand form a duplex structure of at least 12 nucleotides in length but are not covalently linked.

In some embodiments, Lis a tetraloop. In some embodiments, L is 4 nucleotides in length. In some embodiments, L comprises a sequence set forth as GAAA. In some embodiments, each of the A in GAAA sequence is conjugated to a GalNAc moiety. In some embodiments, the G in the GAAA sequence comprises a 2′-O-methyl modification. In some embodiments, the G in the GAAA sequence comprises a 2′-OH. In some embodiments, each of the nucleotide in the GAAA sequence comprises a 2′-O-methyl modification. In some embodiments, each of the A in the GAAA sequence comprises a 2′-OH and the G in the GAAA sequence comprises a 2′-O-methyl modification. In some embodiments, each of the A in the GAAA sequence comprises a 2′-O-methoxyethyl modification and the G in the GAAA sequence comprises a 2′-O-methyl modification. In some embodiments, each of the A in the GAAA sequence comprises a 2′-adem and the G in the GAAA sequence comprises a 2′-O-methyl modification.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 shows the regions of the brain for intraventricular (ICV) administration of RNAi oligonucleotides of interest to a CD-1 mouse (25 g female).

FIG. 2 shows the distribution of Fast Green dye throughout the ventricular system after direct injection of the dye into the right lateral ventricle. 10 μL of FastGreen dye (2.5% in sterile PBS) was delivered at 1 μL/s via 33G Neuros syringe to the right lateral ventricle of a female CD-1 mouse.

FIGS. 3A-3F show the brain injection site for the GalNAc conjugated ALDH2 oligonucleotides (FIG. 3A), and the activity of the oligonucleotides in reducing ALDH2 expression in the liver (FIG. 3B), the hippocampus (FIG. 3C), the somatosensory cortex (FIG. 3D), the striatum (FIG. 3E) and the cerebellum (FIG. 3F). The GalNAc conjugated ALDH2 oligonucleotides were administered via intraventricular administration (100 μg dose, equivalent to 4 mg/kg).

FIG. 4 shows that one single 100 μg dose of GalNAc-conjugated ALDH2 oligonucleotides administered to mice via ICV administration showed similar activities in reducing ALDH2 expression in the cerebellum, compared to a benchmark 900 μg dose (in rat) via intra administration for a different RNAi oligonucleotide (conjugated or unconjugated).

FIG. 5 shows the potency of GalNAc conjugated -ALDH2 oligonucleotides in reducing ALDH2 expression in different brain regions after ICV administration. The remaining ALDH2 mRNA levels were assessed in different brain regions after 5 days (for 100 μg dose) or after 7 days (for 250 μg or 500 μg doses).

FIG. 6 shows the dose response (250 μg or 500 μg) and time course (28 days post administration) of the activities of GalNAc-conjugated ALDH2 oligonucleotides in reducing ALDH2 mRNA expression in various brain regions. The data indicates sustained silencing throughout the brain following a single, ICV injection of the GalNAc-conjugated ALDH2 oligonucleotides.

FIG. 7 shows the dose response (250 μg or 500 μg) and time course (28 days post administration) of the activities of GalNAc-conjugated ALDH2 oligonucleotides in reducing ALDH2 mRNA expression throughout the spinal cord. The data indicates sustained silencing throughout the brain following a single, ICV injection of the GalNAc-conjugated ALDH2 oligonucleotides.

FIG. 8 shows the dose response (100 μg, 250 μg, or 500 μg) and time course (7 days post administration for 100 μg dose, 28 days post administration for 250 μg or 500 μg doses) of the activities of GalNAc-conjugated ALDH2 oligonucleotides in reducing ALDH2 mRNA expression in the liver. The data indicates sustained silencing in the liver following a single administration of the GalNAc-conjugated ALDH2 oligonucleotides.

FIG. 9 shows two-month (56 days) efficacy of GalNAc-conjugated ALDH2 oligonucleotides throughout distinct brain regions after a single, bolus ICV injection (250 μg or 500 μg).

FIG. 10 shows two-month (56 days) efficacy of GalNAc-conjugated ALDH2 oligonucleotides throughout the spinal cord after a single, bolus ICV injection (250 μg or 500 μg).

FIG. 11 show the results of a neurotoxicity study indicating that no glial fibrillary acidic protein (GFAP) upregulation is observed following administration of either 250 or 500 μg of the GalNAc conjugated ALDH2 oligonucleotides. The GalNAc conjugated ALDH2 oligonucleotides did not induce gliosis (a reactive change in glial cells in response to CNS injury).

FIG. 12 shows the activities of the ALDH2 RNAi oligonucleotide derivatives shown in FIG. 23 in reducing ALDH2 expression in the liver after a bolus ICV injection.

FIG. 13 shows activities of the ALDH2 RNAi oligonucleotide derivatives shown in FIG. 23 in reducing ALDH2 expression in various regions of the brain. The data indicates that GalNAc conjugation is not required for efficacy throughout the brain.

FIG. 14 shows the exposure to ALDH2 RNAi oligonucleotide derivatives and ALDH2 mRNA silencing in the frontal cortex following bolus ICV injection. The glia index (glial cell to neuronal cell ratio, also termed “GNR”) in frontal cortex is 1.25.

FIG. 15 shows the exposure to ALDH2 RNAi oligonucleotide derivatives and ALDH2 mRNA silencing in the striatum following bolus ICV injection. The glia index (glial cell to neuronal cell ratio, also termed “GNR”) in striatum varies.

FIG. 16 shows the exposure to ALDH2 RNAi oligonucleotide derivatives and ALDH2 mRNA silencing in the somatosensory cortex following bolus ICV injection. The glia index (glial cell to neuronal cell ratio, also termed “GNR”) in somatosensory cortex is 1.25.

FIG. 17 shows the exposure to ALDH2 RNAi oligonucleotide derivatives and ALDH2 mRNA silencing in the hippocampus following bolus ICV injection. The glia index (glial cell to neuronal cell ratio, also termed “GNR”) in hippocampus is 1.25.

FIG. 18 shows the exposure to ALDH2 RNAi oligonucleotide derivatives and ALDH2 mRNA silencing in hypothalamus following bolus ICV injection. The glia index (glial cell to neuronal cell ratio, also termed “GNR”) in hypothalamus is 1.25.

FIG. 19 shows the exposure to ALDH2 RNAi oligonucleotide derivatives and ALDH2 mRNA silencing in cerebellum following bolus ICV injection. The glia index (glial cell to neuronal cell ratio, also termed “GNR”) in cerebellum 0.25.

FIG. 20 shows a summary of relative exposure ALDH2 RNAi oligonucleotide derivatives across different brain regions.

FIG. 21 shows the exposure to ALDH2 RNAi oligonucleotide derivatives and ALDH2 mRNA silencing across the spinal cord following bolus ICV injection. The glia index (glial cell to neuronal cell ratio, also termed “GNR”) in spinal cord is about 5.

FIG. 22 shows the structures of the different linkers used in the tetraloop of the GalNAc-conjugated ALDH2 oligonucleotides.

FIG. 23 shows the exemplary structures of the oligonucleotide derivatives for use in the CNS. The oligonucleotides shown in the figure target ALDH2.

### DETAILED DESCRIPTION OF THE INVENTION

In some aspects, the disclosure provides oligonucleotides targeting ALDH2 mRNA that are effective for reducing ALDH2 expression in cells, particularly the CNS. The carrier oligonucleotide structure of the invention and the insertion into the CNS will allow the treatment of neurological diseases. Accordingly, in related aspects, the disclosure provides methods of treating neurological diseases by selectively reducing gene expression in the central nervous system. In certain embodiments, ALDH2 targeting oligonucleotides derivatives provided herein are designed for delivery to the cerebrospinal fluid for reducing ALDH2 expression in the central nervous system.

In some embodiments, it is provided herein that, different oligonucleotide size, multimerization and/or molecular weight changes affect the ability of the oligonucleotide to leave CNS. The oligonucleotides will selectively function in the nuclease-lite CNS. Though the oligonucleotides can eventually enter the lymphatic system from the CNS, they will be degraded as they enter a nuclease-rich environment, thus preventing off target effects outside of the CNS. This effectively allows the engineering of a “kill switch” that will allow activity in the CNS and prevent off-target effects in other tissues.

Further aspects of the disclosure, including a description of defined terms, are provided below.

### I. Definitions

ALDH2: As used herein, the term, “ALDH2” refers to the aldehyde dehydrogenase 2 family (mitochondrial) gene. ALDH2 encodes proteins that belong to the aldehyde dehydrogenase family of proteins and function as the second enzyme of the oxidative pathway of alcohol metabolism that synthesizes acetate (acetic acid) from ethanol. Homologs of ALDH2 are conserved across a range of species, including human, mouse, rat, non-human primate species, and others (see, e.g., NCBI HomoloGene:55480). ALDH2 also has homology to other aldehyde dehydrogenase encoding genes, including, for example, ALDH1A1. In humans, ALDH2 encodes at least two transcripts, namely NM_000690.3 (variant 1) and NM_001204889.1 (variant 2), each encoding a different isoform, NP_000681.2 (isoform 1) and NP_001191818.1 (isoform 2), respectively. Transcript variant 2 lacks an in-frame exon in the 5′ coding region, compared to transcript variant 1, and encodes a shorter isoform (2), compared to isoform 1. Polymorphisms in ALDH2 have been identified (see, e.g., Chang et al., “ALDH2 polymorphism and alcohol-related cancers in Asians: a public health perspective,” J Biomed Sci., 2017, 24(1):19. Review).

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Administering: As used herein, the terms “administering” or “administration” means to provide a substance (e.g., an oligonucleotide) to a subject in a manner that is pharmacologically useful (e.g., to treat a condition in the subject). In some embodiments, the oligonucleotides of the present disclosure are administered to the cerebrospinal fluid of a subject, e.g., via intraventricular, intracavitary, intrathecal, or interstitial injection or infusion. This is particularly true for neurodegenerative diseases like ALS, Huntington's Disease, Alzheimer's Disease or the like. The compounds can also be administered by transfection or infection using methods known in the art, including but not limited to the methods described in McCaffrey et al., Nature, 2002, 418(6893):38-9 (hydrodynamic transfection), or Xia et al., Nature Biotechnol., 2002, 20(10):1006-10 (viral-mediated delivery);

Cerebrospinal fluid: As used herein, the term “cerebrospinal fluid” refers to the fluid surrounding the brain and spinal cord. Cerebrospinal fluid generally occupies space between the arachnoid membrane and the pia mater. Additionally, cerebrospinal fluid is generally understood to be produced by ependymal cells in the choroid plexuses of the ventricles of the brain and absorbed in the arachnoid granulations.

Complementary: As used herein, the term “complementary” refers to a structural relationship between nucleotides (e.g., two nucleotide on opposing nucleic acids or on opposing regions of a single nucleic acid strand) that permits the nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have nucleotide sequences that are complementary to each other so as to form regions of complementarity, as described herein.

Deoxyribonucleotide: As used herein, the term “deoxyribonucleotide” refers to a nucleotide having a hydrogen at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

Double-stranded oligonucleotide: As used herein, the term “double-stranded oligonucleotide” refers to an oligonucleotide that is substantially in a duplex form. In some embodiments, complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from a single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed, e.g., having overhangs at one or both ends. In some embodiments, a double-stranded oligonucleotide comprises antiparallel sequences of nucleotides that are partially complementary, and thus, may have one or more mismatches, which may include internal mismatches or end mismatches.

Duplex: As used herein, the term “duplex,” in reference to nucleic acids (e.g., oligonucleotides), refers to a structure formed through complementary base-pairing of two antiparallel sequences of nucleotides.

Excipient: As used herein, the term “excipient” refers to a non-therapeutic agent that may be included in a composition, for example, to provide or contribute to a desired consistency or stabilizing effect.

Loop: As used herein, the term “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

Modified Internucleotide Linkage: As used herein, the term “modified internucleotide linkage” refers to an internucleotide linkage having one or more chemical modifications compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

Modified Nucleotide: As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modifications in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc. In certain embodiments, a modified nucleotide comprises a 2′-O-methyl or a 2′-F substitution at the 2′ position of the ribose ring.

Nicked Tetraloop Structure: A “nicked tetraloop structure” is a structure of a RNAi oligonucleotide characterized by the presence of separate sense (passenger) and antisense (guide) strands, in which the sense strand has a region of complementarity to the antisense strand such that the two strands form a duplex, and in which at least one of the strands, generally the sense strand, extends from the duplex in which the extension contains a tetraloop and two self-complementary sequences forming a stem region adjacent to the tetraloop, in which the tetraloop is configured to stabilize the adjacent stem region formed by the self-complementary sequences of the at least one strand.

Oligonucleotide: As used herein, the term “oligonucleotide” refers to a short nucleic acid, e.g., of less than 100 nucleotides in length. An oligonucleotide can comprise ribonucleotides, deoxyribonucleotides, and/or modified nucleotides including, for example, modified ribonucleotides. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

Overhang: As used herein, the term “overhang” refers to terminal non-base-pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotide.

Phosphate analog: As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., PCT publication WO2018045317, filed on Sep. 1, 2017, U.S. Provisional Application numbers 62/383,207, filed on Sep. 2, 2016, and 62/393,401, filed on Sep. 12, 2016, the contents of each of which relating to phosphate analogs are incorporated herein by reference. Other modifications have been developed for the 5′ end of oligonucleotides (see, e.g., WO 2011/133871; U.S. Pat. No. 8,927,513; and Prakash et al., Nucleic Acids Res., 2015, 43(6):2993-3011, the contents of each of which relating to phosphate analogs are incorporated herein by reference).

Reduced expression: As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with a double-stranded oligonucleotide (e.g., one having an antisense strand that is complementary to ALDH2 mRNA sequence) may result in a decrease in the amount of RNA transcript, protein and/or enzymatic activity (e.g., encoded by the ALDH2 gene) compared to a cell that is not treated with the double-stranded oligonucleotide. Similarly, “reducing expression” as used herein refers to an act that results in reduced expression of a gene (e.g., ALDH2).

Region of Complementarity: As used herein, the term “region of complementarity” refers to a sequence of nucleotides of a nucleic acid (e.g., a double-stranded oligonucleotide) that is sufficiently complementary to an antiparallel sequence of nucleotides (e.g., a target nucleotide sequence within an mRNA) to permit hybridization between the two sequences of nucleotides under appropriate hybridization conditions, e.g., in a phosphate buffer, in a cell, etc. A region of complementarity may be fully complementary to a nucleotide sequence (e.g., a target nucleotide sequence present within an mRNA or portion thereof). For example, a region of complementary that is fully complementary to a nucleotide sequence present in an mRNA has a contiguous sequence of nucleotides that is complementary, without any mismatches or gaps, to a corresponding sequence in the mRNA. Alternatively, a region of complementarity may be partially complementary to a nucleotide sequence (e.g., a nucleotide sequence present in an mRNA or portion thereof). For example, a region of complementary that is partially complementary to a nucleotide sequence present in an mRNA has a contiguous sequence of nucleotides that is complementary to a corresponding sequence in the mRNA but that contains one or more mismatches or gaps (e.g., 1, 2, 3, or more mismatches or gaps) compared with the corresponding sequence in the mRNA, provided that the region of complementarity remains capable of hybridizing with the mRNA under appropriate hybridization conditions.

Ribonucleotide: As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

RNAi Oligonucleotide: As used herein, the term “RNAi oligonucleotide” refers to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA.

Strand: As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

Subject: As used herein, the term “subject” means any mammal, including mice, rabbits, and humans. In one embodiment, the subject is a human or non-human primate. The terms “individual” or “patient” may be used interchangeably with “subject.”

Synthetic: As used herein, the term “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

Targeting ligand: As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

Tetraloop: As used herein, the term “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base-pairing, stacking interactions, hydrogen bonding, and contact interactions (Cheong et al., Nature, 1990, 346(6285):680-2; Heus and Pardi, Science, 1991, 253(5016):191-4). In some embodiments, a tetraloop comprises or consists of 3 to 6 nucleotides and is typically 4 to 5 nucleotides. In certain embodiments, a tetraloop comprises or consists of three, four, five, or six nucleotides, which may or may not be modified (e.g., which may or may not be conjugated to a targeting moiety). In one embodiment, a tetraloop consists of four nucleotides. Any nucleotide may be used in the tetraloop and standard IUPAC-IUB symbols for such nucleotides may be used as described in Cornish-Bowden, Nucl. Acids Res., 1985, 13:3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., Proc Natl Acad Sci USA., 1990, 87(21):8467-71; Antao et al., Nucleic Acids Res., 1991, 19(21):5901-5). Examples of DNA tetraloops include the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)). See, for example: Nakano et al., Biochemistry, 2002, 41 (48):14281-292; Shinji et al., Nippon Kagakkai Koen Yokoshu, 2000, 78(2):731, which are incorporated by reference herein for their relevant disclosures. In some embodiments, the tetraloop is contained within a nicked tetraloop structure.

Treat: As used herein, the term “treat” refers to the act of providing care to a subject in need thereof, e.g., through the administration a therapeutic agent (e.g., an oligonucleotide) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### II. Oligonucleotide-Based Inhibitors

i. ALDH2 Targeting Oligonucleotides

Oligonucleotides potent in the CNS are provided herein that were identified through examination of the ALDH2 mRNA, including mRNAs of multiple different species (human, cynomolgus monkey, and mouse), and in vitro and in vivo testing. As described herein, such oligonucleotides can be used to achieve therapeutic benefit for subjects having neurological diseases (e.g., neurodegenerative diseases, cognitive disorders, or anxiety disorders) by reducing gene activity (e.g., in the central nervous system), in this case the activity of ALDH2. Other genes that could be targeted with the methods and oligonucleotides of the current invention include those identified as causing: Spinocerebellar Ataxia Type 1 (Ataxin-1, and/or Ataxin-3); the β-amyloid precursor protein gene (APP or BACE1) or mutants thereof; Dystonia (DYT1); Amyotrophic Lateral Sclerosis “ALS” or Lou Gehrig's Disease (SOD1), and various genes that lead to tumors in the CNS. For example, potent RNAi oligonucleotides are provided herein that have a sense strand comprising, or consisting of, a sequence as set forth in any one of SEQ ID NO: 581-590, 608, and 609 and an antisense strand comprising, or consisting of, a complementary sequence selected from SEQ ID NO: 591-600, as is also arranged the table provided in Appendix A (e.g., a sense strand comprising a sequence as set forth in SEQ ID NO: 585 and an antisense strand comprising a sequence as set forth in SEQ ID NO: 595).

The sequences can be put into multiple different oligonucleotide structures (or formats). For example, in some embodiments, the sequences can be incorporated into oligonucleotides that comprise sense and antisense strands that are both in the range of 17 to 36 nucleotides in length. In some embodiments, oligonucleotides incorporating such sequences are provided that have a tetraloop structure within a 3′ extension of their sense strand, and two terminal overhang nucleotides at the 3′ end of its antisense strand. In some embodiments, the two terminal overhang nucleotides are GG. Typically, one or both of the two terminal GG nucleotides of the antisense strand is or are not complementary to the target.

In some embodiments, oligonucleotides incorporating such sequences are provided that have sense and antisense strands that are both in the range of 21 to 23 nucleotides in length. In some embodiments, a 3′ overhang is provided on the sense, antisense, or both sense and antisense strands that is 1 or 2 nucleotides in length. In some embodiments, an oligonucleotide has a guide strand of 23 nucleotides and a passenger strand of 21 nucleotides, in which the 3′-end of passenger strand and 5′-end of guide strand form a blunt end and where the guide strand has a two nucleotide 3′ overhang. In some embodiments, a 3′ overhang is provided on the antisense strand that is 9 nucleotides in length. For example, an oligonucleotide provided herein may have a guide strand of 22 nucleotides and a passenger strand of 29 nucleotides, wherein the passenger strand forms a tetraloop structure at the 3′ end and the guide strand has a 9 nucleotide 3′ overhang (herein termed “N-9”).

In some embodiments, it has been discovered that certain regions of ALDH2 mRNA are hotspots for targeting because they are more amenable than other regions to oligonucleotide-based inhibition. In some embodiments, a hotspot region of ALDH2 comprises, or consists of, a sequence as forth in any one of SEQ ID NOs: 601-607. These regions of ALDH2 mRNA may be targeted using oligonucleotides as discussed herein for purposes of inhibiting ALDH2 mRNA expression.

Accordingly, in some embodiments, oligonucleotides provided herein are designed to have regions of complementarity to ALDH2 mRNA (e.g., within a hotspot of ALDH2 mRNA) for purposes of targeting the mRNA in cells and inhibiting its expression. The region of complementarity is generally of a suitable length and base content to enable annealing of the oligonucleotide (or a strand thereof) to ALDH2 mRNA for purposes of inhibiting its expression.

In some embodiments, an oligonucleotide disclosed herein comprises a region of complementarity (e.g., on an antisense strand of a double-stranded oligonucleotide) that is at least partially complementary to a sequence of interest in a target gene. According to the current invention such sequences are as set forth in SEQ ID NOs: 1-14 and 17-290, which include sequences mapping to within hotspot regions of ALDH2 mRNA. In some embodiments, an oligonucleotide disclosed herein comprises a region of complementarity (e.g., on an antisense strand of a double-stranded oligonucleotide) that is fully complementary to a sequence as set forth in SEQ ID NOs: 1-14 and 17-290. In some embodiments, a region of complementarity of an oligonucleotide that is complementary to contiguous nucleotides of a sequence as set forth in SEQ ID NOs: 1-14 and 17-290 spans the entire length of an antisense strand. In some embodiments, a region of complementarity of an oligonucleotide that is complementary to contiguous nucleotides of a sequence as set forth in any one of SEQ ID NOs: 1-14 and 17-290 spans a portion of the entire length of an antisense strand (e.g., all but two nucleotides at the 3′ end of the antisense strand). In some embodiments, an oligonucleotide disclosed herein comprises a region of complementarity (e.g., on an antisense strand of a double-stranded oligonucleotide) that is at least partially (e.g., fully) complementary to a contiguous stretch of nucleotides spanning nucleotides 1-19 of a sequence as set forth in SEQ ID NOs: 581-590.

In some embodiments, the region of complementarity is at least 12, at least 13, at least 14, at least 15, at least 16, at least 17, at least 18, at least 19, at least 20, at least 21, at least 22, at least 23, at least 24, at least 25 nucleotides in length. In some embodiments, an oligonucleotide provided herein has a region of complementarity to ALDH2 that is in the range of 12 to 30 (e.g., 12 to 30, 12 to 22, 15 to 25, 17 to 21, 18 to 27, 19 to 27, or 15 to 30) nucleotides in length. In some embodiments, an oligonucleotide provided herein has a region of complementarity to ALDH2 that is 12, 13, 14, 15, 16, 17, 18, 19, 20, 21, 22, 23, 24, 25, 26, 27, 28, 29 or 30 nucleotides in length.

In some embodiments, a region of complementarity to ALDH2 may have one or more mismatches compared with a corresponding sequence of ALDH2 mRNA. A region of complementarity on an oligonucleotide may have up to 1, up to 2, up to 3, up to 4, up to 5, etc., mismatches provided that it maintains the ability to form complementary base pairs with ALDH2 mRNA under appropriate hybridization conditions. Alternatively, a region of complementarity on an oligonucleotide may have no more than 1, no more than 2, no more than 3, no more than 4, or no more than 5 mismatches provided that it maintains the ability to form complementary base pairs with ALDH2 mRNA under appropriate hybridization conditions. In some embodiments, if there are more than one mismatches in a region of complementarity, they may be positioned consecutively (e.g., 2, 3, 4, or more in a row), or interspersed throughout the region of complementarity provided that the oligonucleotide maintains the ability to form complementary base pairs with ALDH2 mRNA under appropriate hybridization conditions.

In some embodiments, double-stranded oligonucleotides provided herein comprise, or consist of, a sense strand having a sequence as set forth in any one of SEQ ID NO: 1-14 and 17-290 and an antisense strand comprising a complementary sequence selected from SEQ ID NO: 291-304 and 307-580, as is arranged in the table provided in Appendix A (e.g., a sense strand comprising a sequence as set forth in SEQ ID NO: 1 and an antisense strand comprising a sequence as set forth in SEQ ID NO: 291).

ii. Oligonucleotide Structures

There are a variety of structures of oligonucleotides that are useful for targeting ALDH2 in the methods of the present disclosure, including RNAi, miRNA, etc. Any of the structures described herein or elsewhere may be used as a framework to incorporate or target a sequence described herein (e.g., a hotpot sequence of ALDH2 such as those illustrated in SEQ ID NOs: 601-607). Double-stranded oligonucleotides for targeting ALDH2 expression (e.g., via the RNAi pathway) generally have a sense strand and an antisense strand that form a duplex with one another. In some embodiments, the sense and antisense strands are not covalently linked. However, in some embodiments, the sense and antisense strands are covalently linked.

In some embodiments, double-stranded oligonucleotides for reducing the expression of ALDH2 expression engage RNA interference (RNAi). For example, RNAi oligonucleotides have been developed with each strand having sizes of 19-25 nucleotides with at least one 3′ overhang of 1 to 5 nucleotides (see, e.g., U.S. Pat. No. 8,372,968). Longer oligonucleotides have also been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as WO2010033225, which are incorporated by reference herein for their disclosure of these oligonucleotides). Such structures may include single-stranded extensions (on one or both sides of the molecule) as well as double-stranded extensions.

In some embodiments, oligonucleotides may be in the range of 21 to 23 nucleotides in length. In some embodiments, oligonucleotides may have an overhang (e.g., of 1, 2, or 3 nucleotides in length) in the 3′ end of the sense and/or antisense strands. In some embodiments, oligonucleotides (e.g., siRNAs) may comprise a 21-nucleotide guide strand that is antisense to a target RNA and a complementary passenger strand, in which both strands anneal to form a 19-bp duplex and 2 nucleotide overhangs at either or both 3′ ends. In some embodiments, oligonucleotides (e.g., siRNAs) may comprise a 22-nucleotide guide strand that is antisense to a target RNA and a complementary passenger strand, in which both strands anneal to form a 13-bp duplex and 9 nucleotide overhangs at either or both 3′ ends. See, for example, U.S. Pat. Nos. 9,012,138; 9,012,621, and 9,193,753, the contents of each of which are incorporated herein for their relevant disclosures.

In some embodiments, an oligonucleotide of the invention has a 36-nucleotide sense strand that comprises a region extending beyond the antisense-sense duplex, where the extension region has a stem-tetraloop structure where the stem is a six base pair duplex and where the tetraloop has four nucleotides. In certain of those embodiments, three or four of the tetraloop nucleotides are each conjugated to a monovalent GalNac ligand. In certain of those embodiments, all of the tetraloop nucleotides are each conjugated to a monovalent GalNac ligand.

In some embodiments, an oligonucleotide of the invention comprises a 25-nucleotide sense strand and a 27-nucleotide antisense strand that when acted upon by a dicer enzyme results in an antisense strand that is incorporated into the mature RISC.

Other oligonucleotide designs for use with the compositions and methods are disclosed herein include: 16-mer siRNAs (see, e.g., Nucleic Acids in Chemistry and Biology. Blackburn (ed.), Royal Society of Chemistry, 2006), shRNAs (e.g., having 19 bp or shorter stems; see, e.g., Moore et al., Methods Mol. Biol., 2010, 629:141-158), blunt siRNAs (e.g., of 19 bps in length; see, e.g., Kraynack and Baker, R N A, 2006, 12:163-176), asymmetrical siRNAs (aiRNA; see, e.g., Sun et al., Nat. Biotechnol., 2008, 26:1379-1382), asymmetric shorter-duplex siRNA (see, e.g., Chang et al., Mol Ther., 2009, 17(4):725-32), fork siRNAs (see, e.g., Hohjoh, FEBS Letters, 2004, 557(1-3):193-198), single-stranded siRNAs (Elsner et al., Nature Biotechnology, 2012, 30:1063), dumbbell-shaped circular siRNAs (see, e.g., Abe et al., J Am Chem Soc., 2007, 129:15108-15109), and small internally segmented interfering RNA (sisiRNA; see, e.g., Bramsen et al., Nucleic Acids Res., 2007, 35(17):5886-5897). Each of the foregoing references is incorporated by reference in its entirety for the related disclosures therein. Further non-limiting examples of an oligonucleotide structures that may be used in some embodiments to reduce or inhibit the expression of ALDH2 are microRNA (miRNA), short hairpin RNA (shRNA), and short siRNA (see, e.g., Hamilton et al., EMBO J., 2002, 21(17):4671-4679; see also U.S. Application No. 20090099115).

a. Antisense Strands

In some embodiments, an oligonucleotide disclosed herein for targeting ALDH2 comprises an antisense strand comprising or consisting of a sequence as set forth in any one of SEQ ID NOs: 291-304, 307-580 and 591-600. In some embodiments, an oligonucleotide comprises an antisense strand comprising or consisting of at least 12 (e.g., at least 12, at least 13, at least 14, at least 15, at least 16, at least 17, at least 18, at least 19, at least 20, at least 21, at least 22, or at least 23) contiguous nucleotides of a sequence as set forth in any one of SEQ ID NOs: 291-304, 307-580 and 591-600.

In some embodiments, a double-stranded oligonucleotide may have an antisense strand of up to 40 nucleotides in length (e.g., up to 40, up to 35, up to 30, up to 27, up to 25, up to 21, up to 19, up to 17, or up to 12 nucleotides in length). In some embodiments, an oligonucleotide may have an antisense strand of at least 12 nucleotides in length (e.g., at least 12, at least 15, at least 19, at least 21, at least 25, at least 27, at least 30, at least 35, or at least 38 nucleotides in length). In some embodiments, an oligonucleotide may have an antisense strand in a range of 12 to 40 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40, or 32 to 40) nucleotides in length. In some embodiments, an oligonucleotide may have an antisense strand in a range of 19-27 (e.g., 19 to 27, 19-25, 19-23, 19-21, 21-27, 21-25, 21-23, 23-27, 23-25, or 25-27) nucleotides in length. In some embodiments, an oligonucleotide may have an antisense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, an antisense strand of an oligonucleotide may be referred to as a “guide strand.” For example, if an antisense strand can engage with RNA-induced silencing complex (RISC) and bind to an Argonaut protein, or engage with or bind to one or more similar factors, and direct silencing of a target gene, it may be referred to as a guide strand. In some embodiments, a sense strand complementary to a guide strand may be referred to as a “passenger strand.”

b. Sense Strands

In some embodiments, an oligonucleotide disclosed herein for targeting ALDH2 comprises or consists of a sense strand sequence as set forth in any one of SEQ ID NOs: 1-14, 17-290, 581-590, 608, and 609. In some embodiments, an oligonucleotide has a sense strand that comprises or consists of at least 12 (e.g., at least 13, at least 14, at least 15, at least 16, at least 17, at least 18, at least 19, at least 20, at least 21, at least 22, or at least 23) contiguous nucleotides of a sequence as set forth in any one of SEQ ID NOs: 1-14, 17-290, 581-590, 608, and 609.

In some embodiments, an oligonucleotide may have a sense strand (or passenger strand) of up to 40 nucleotides in length (e.g., up to 40, up to 35, up to 30, up to 27, up to 25, up to 21, up to 19, up to 17, or up to 12 nucleotides in length). In some embodiments, an oligonucleotide may have a sense strand of at least 12 nucleotides in length (e.g., at least 12, at least 15, at least 19, at least 21, at least 25, at least 27, at least 30, at least 35, or at least 38 nucleotides in length). In some embodiments, an oligonucleotide may have a sense strand in a range of 12 to 40 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40, or 32 to 40) nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, a sense strand comprises a stem-loop structure at its 3′-end. In some embodiments, a sense strand comprises a stem-loop structure at its 5′-end. In some embodiments, a stem is a duplex of 2, 3, 4, 5, 6, 7, 8, 9, 10, 11, 12, 13, or 14 nucleotides in length. In some embodiments, a stem-loop provides the molecule better protection against degradation (e.g., enzymatic degradation) and facilitates targeting characteristics for delivery to a target cell. For example, in some embodiments, a loop provides added nucleotides on which modification can be made without substantially affecting the gene expression inhibition activity of an oligonucleotide. In certain embodiments, an oligonucleotide is provided herein in which the sense strand comprises (e.g., at its 3′-end) a stem-loop set forth as: S₁-L-S₂, in which S₁ is complementary to S₂, and in which L forms a loop between S₁ and S₂ of up to 10 nucleotides in length (e.g., 3, 4, 5, 6, 7, 8, 9, or 10 nucleotides in length).

In some embodiments, a loop (L) of a stem-loop is a tetraloop (e.g., within a nicked tetraloop structure). A tetraloop may contain ribonucleotides, deoxyribonucleotides, modified nucleotides, and combinations thereof. Typically, a tetraloop has 4 to 5 nucleotides. In some embodiments, the loop (L) comprises a sequence set forth as GAAA.

c. Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 20, 21, 22, 23, 24, 25, 26, 27, 28, 29, or 30 nucleotides in length. In some embodiments a duplex formed between a sense and antisense strand does not span the entire length of the sense strand and/or antisense strand. In some embodiments, a duplex between a sense and antisense strand spans the entire length of either the sense or antisense strands. In certain embodiments, a duplex between a sense and antisense strand spans the entire length of both the sense strand and the antisense strand.

d. Oligonucleotide Ends

In some embodiments, an oligonucleotide provided herein comprises sense and antisense strands, such that there is a 3′-overhang on either the sense strand or the antisense strand, or both the sense and antisense strand. In some embodiments, oligonucleotides provided herein have one 5′ end that is thermodynamically less stable compared to the other 5′ end. In some embodiments, an asymmetric oligonucleotide is provided that includes a blunt end at the 3′ end of a sense strand and an overhang at the 3′ end of an antisense strand. In some embodiments, a 3′ overhang on an antisense strand is 1-8 nucleotides in length (e.g., 1, 2, 3, 4, 5, 6, 7 or 8 nucleotides in length).

Typically, an oligonucleotide for RNAi has a two-nucleotide overhang on the 3′ end of the antisense (guide) strand. However, other overhangs are possible. In some embodiments, an overhang is a 3′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides. However, in some embodiments, the overhang is a 5′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides.

In some embodiments, an oligonucleotide of the present disclosure has a nine nucleotide overhang on the 3′ end of the antisense (guide) strand (referred to herein as “N9”). An exemplary N9 oligonucleotide comprises a sense strand having a sequence set forth in SEQ ID NO: 608 and an antisense strand having a sequence set forth in SEQ ID NO: 595.

In some embodiments, one or more (e.g., 2, 3, 4) terminal nucleotides of the 3′ end or 5′ end of a sense and/or antisense strand are modified. For example, in some embodiments, one or two terminal nucleotides of the 3′ end of an antisense strand are modified. In some embodiments, the last nucleotide at the 3′ end of an antisense strand is modified, e.g., comprises 2′-modification, such as a 2′-O-methoxyethyl. In some embodiments, the last one or two terminal nucleotides at the 3′ end of an antisense strand are complementary to the target. In some embodiments, the last one or two nucleotides at the 3′ end of the antisense strand are not complementary to the target. In some embodiments, the 5′ end and/or the 3′ end of a sense or antisense strand has an inverted cap nucleotide.

e. Mismatches

In some embodiments, the oligonucleotide has one or more (e.g., 1, 2, 3, 4, 5) mismatches between a sense and antisense strand. If there is more than one mismatch between a sense and antisense strand, they may be positioned consecutively (e.g., 2, 3 or more in a row), or interspersed throughout the region of complementarity. In some embodiments, the 3′-terminus of the sense strand contains one or more mismatches. In one embodiment, two mismatches are incorporated at the 3′ terminus of the sense strand. In some embodiments, base mismatches or destabilization of segments at the 3′-end of the sense strand of the oligonucleotide improved the potency of synthetic duplexes in RNAi, possibly through facilitating processing by Dicer.

iii. Single-Stranded Oligonucleotides

In some embodiments, an oligonucleotide for reducing ALDH2 expression as described herein is single-stranded. Such structures may include but are not limited to single-stranded RNAi oligonucleotides. Recent efforts have demonstrated the activity of single-stranded RNAi oligonucleotides (see, e.g., Matsui et al., Molecular Therapy, 2016, 24(5):946-955). However, in some embodiments, oligonucleotides provided herein are antisense oligonucleotides (ASOs). An antisense oligonucleotide is a single-stranded oligonucleotide that has a nucleobase sequence which, when written in the 5′ to 3′ direction, comprises the reverse complement of a targeted segment of a particular nucleic acid and is suitably modified (e.g., as a gapmer) so as to induce RNaseH mediated cleavage of its target RNA in cells or (e.g., as a mixmer) so as to inhibit translation of the target mRNA in cells. Antisense oligonucleotides for use in the instant disclosure may be modified in any suitable manner known in the art including, for example, as shown in U.S. Pat. No. 9,567,587, which is incorporated by reference herein for its disclosure regarding modification of antisense oligonucleotides (including, e.g., length, sugar moieties of the nucleobase (pyrimidine, purine), and alterations of the heterocyclic portion of the nucleobase). Further, antisense molecules have been used for decades to reduce expression of specific target genes (see, e.g., Bennett et al., Pharmacology of Antisense Drugs, Annual Review of Pharmacology and Toxicology, 2017, 57:81-105).

iv. Oligonucleotide Modifications

Oligonucleotides may be modified in various ways to improve or control specificity, stability, delivery, bioavailability, resistance from nuclease degradation, immunogenicity, base-paring properties, RNA distribution and cellular uptake and other features relevant to therapeutic or research use. See, e.g., Bramsen et al., Nucleic Acids Res., 2009, 37:2867-2881; Bramsen and Kjems, Frontiers in Genetics, 2012, 3:1-22). Accordingly, in some embodiments, oligonucleotides of the present disclosure may include one or more suitable modifications. In some embodiments, a modified nucleotide has a modification in its base (or nucleobase), the sugar (e.g., ribose, deoxyribose), or the phosphate group.

The number of modifications on an oligonucleotide and the positions of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier (e.g., “naked delivery”), it may be advantageous for at least some of the nucleotides to be modified. Accordingly, in certain embodiments of any of the oligonucleotides provided herein, all or substantially all the nucleotides of an oligonucleotide are modified. In certain embodiments, more than half of the nucleotides are modified. In certain embodiments, less than half of the nucleotides are modified. Typically, with naked delivery, every sugar is modified at the 2′-position. These modifications may be reversible or irreversible. In some embodiments, an oligonucleotide as disclosed herein has a number and type of modified nucleotides sufficient to cause the desired characteristic (e.g., protection from enzymatic degradation, capacity to target a desired cell after in vivo administration, and/or thermodynamic stability).

a. Sugar Modifications

In some embodiments, a modified sugar (also referred to herein as a sugar analog) includes a modified deoxyribose or ribose moiety, e.g., in which one or more modifications occur at the 2′, 3′, 4′, and/or 5′ carbon position of the sugar. In some embodiments, a modified sugar may also include non-natural alternative carbon structures such as those present in locked nucleic acids (“LNA”) (see, e.g., Koshkin et al., Tetrahedron, 1998, 54:3607-3630), unlocked nucleic acids (“UNA”) (see, e.g., Snead et al., Molecular Therapy—Nucleic Acids, 2013, 2:e103), and bridged nucleic acids (“BNA”) (see, e.g., Imanishi and Obika, The Royal Society of Chemistry, Chem. Commun., 2002, 1653-1659); Koshkin et al., Snead et al., and Imanishi and Obika are incorporated by reference herein for their disclosures relating to sugar modifications.

In some embodiments, a nucleotide modification in a sugar comprises a 2′-modification. In certain embodiments, the 2′-modification may be 2′-aminoethyl, 2′-fluoro, 2′-O-methyl, 2′-O-methoxyethyl, or 2′-deoxy-2′-fluoro-β-d-arabinonucleic acid. Typically, the modification is 2′-fluoro, 2′-O-methyl, 2′-O-methoxyethyl, 2′-adem, or 2′-aminodiethoxymethanol. However, a large variety of 2′ position modifications that have been developed for use in oligonucleotides can be employed in oligonucleotides disclosed herein. See, e.g., Bramsen et al., Nucleic Acids Res., 2009, 37:2867-2881. In some embodiments, a modification in a sugar comprises a modification of the sugar ring, which may comprise modification of one or more carbons of the sugar ring. For example, a modification of a sugar of a nucleotide may comprise a linkage between the 2′-carbon and a 1′-carbon or 4′-carbon of the sugar. For example, the linkage may comprise an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the terminal 3′-end group (e.g., a 3′-hydroxyl) is a phosphate group or other group, which can be used, for example, to attach linkers, adapters or labels or for the direct ligation of an oligonucleotide to another nucleic acid.

b. 5′ Terminal Phosphates

5′-terminal phosphate groups of oligonucleotides may or in some circumstances enhance the interaction with Argonaut 2. However, oligonucleotides comprising a 5′-phosphate group may be susceptible to degradation via phosphatases or other enzymes, which can limit their bioavailability in vivo. In some embodiments, oligonucleotides include analogs of 5′ phosphates that are resistant to such degradation. In some embodiments, a phosphate analog may be oxymethylphosphonate, vinylphosphonate, or malonylphosphonate. In certain embodiments, the 5′ end of an oligonucleotide strand is attached to a chemical moiety that mimics the electrostatic and steric properties of a natural 5′-phosphate group (“phosphate mimic”) (see, e.g., Prakash et al., Nucleic Acids Res., 2015, 43(6):2993-3011, the contents of which relating to phosphate analogs are incorporated herein by reference). Many phosphate mimics have been developed that can be attached to the 5′ end (see, e.g., U.S. Pat. No. 8,927,513, the contents of which relating to phosphate analogs are incorporated herein by reference). Other modifications have been developed for the 5′ end of oligonucleotides (see, e.g., WO 2011/133871, the contents of which relating to phosphate analogs are incorporated herein by reference). In certain embodiments, a hydroxyl group is attached to the 5′ end of the oligonucleotide.

In some embodiments, an oligonucleotide has a phosphate analog at a 4′-carbon position of the sugar (referred to as a “4′-phosphate analog”). See, for example, International Patent publication WO2018045317; U.S. Provisional Application numbers 62/383,207, entitled 4′-Phosphate Analogs and Oligonucleotides Comprising the Same, filed on Sep. 2, 2016, and 62/393,401, filed on Sep. 12, 2016, entitled 4′-Phosphate Analogs and Oligonucleotides Comprising the Same, the contents of each of which relating to phosphate analogs are incorporated herein by reference. In some embodiments, an oligonucleotide provided herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the aminomethyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂S₁ (CH₃)₃, or a protecting group. In certain embodiments, the alkyl group is CH₂CH₃. More typically, R is independently selected from H, CH₃, or CH₂CH₃.

c. Modified Internucleoside Linkages

In some embodiments, the oligonucleotide may comprise a modified internucleoside linkage. In some embodiments, phosphate modifications or substitutions may result in an oligonucleotide that comprises at least one (e.g., at least 1, at least 2, at least 3 or at least 5) modified internucleotide linkage. In some embodiments, any one of the oligonucleotides disclosed herein comprises 1 to 12 (e.g., 1 to 12, 1 to 10, 2 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9, 10, 11, or 12 modified internucleotide linkages.

A modified internucleotide linkage may be a phosphorodithioate linkage, a phosphorothioate linkage, a phosphotriester linkage, a thionoalkylphosphonate linkage, a thionoalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, in the N9 oligonucleotides, each of the internucleoside linkage in the 9 nucleotide 3′ overhang is a modified internucleotide linkage (e.g., a phosphorothioate linkage).

d. Base Modifications

In some embodiments, oligonucleotides provided herein have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain a nitrogen atom. See, e.g., U.S. Published Patent Application No. 20080274462. In some embodiments, a modified nucleotide comprises a universal base. However, in certain embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution that, when present in a duplex, can be positioned opposite more than one type of base without substantially altering the structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include inosine, 1-β-D-ribofuranosyl-5-nitroindole, and/or 1-β-D-ribofuranosyl-3-nitropyrrole (US Pat. Appl. Publ. No. 20070254362 to Quay et al.; Van Aerschot et al., Nucleic Acids Res., 1995, 23(21):4363-70; Loakes et al., Nucleic Acids Res., 1995, 23(13):2361-6; Loakes and Brown, Nucleic Acids Res., 1994, 22(20):4039-43). Each of the foregoing is incorporated by reference herein for their disclosures relating to base modifications).

e. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See U.S. Published Application No. 2011/0294869 originally assigned to Traversa Therapeutics, Inc. (“Traversa”); PCT Publication No. WO 2015/188197 to Solstice Biologics, Ltd. (“Solstice”); Meade et al., Nature Biotechnology, 2014, 32:1256-1263; PCT Publication No. WO 2014/088920 to Merck Sharp & Dohme Corp.; each of which are incorporated by reference for their disclosures of such modifications. This reversible modification of the internucleotide diphosphate linkages is designed to be cleaved intracellularly by the reducing environment of the cytosol (e.g., glutathione). Earlier examples include neutralizing phosphotriester modifications that were reported to be cleavable inside cells (Dellinger et al., J. Am. Chem. Soc., 2003, 125:940-950).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione sensitive moieties, it is possible to introduce sterically larger chemical groups into the oligonucleotide of interest as compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of a sugar, particularly when the modified nucleotide is the 3′-terminal nucleotide of the oligonucleotide. In some embodiments, the glutathione-sensitive moiety comprises a sulfonyl group. See, e.g., PCT publication WO2018039364, and U.S. Provisional Application No. 62/378,635, entitled Compositions Comprising Reversibly Modified Oligonucleotides and Uses Thereof, filed on Aug. 23, 2016, the contents of which are incorporated by reference herein for its relevant disclosures.

v. Targeting Ligands

In some embodiments, it may be desirable to target the oligonucleotides of the disclosure to one or more cells or cell types of the CNS where reduction of mutant or toxic gene expression may provide clinical benefit. Such a strategy may help to avoid undesirable effects in other organs or cell types, or may avoid undue loss of the oligonucleotide to cells, tissue or organs that would not benefit from the inhibitory aspects of the oligonucleotide. Accordingly, in some embodiments, oligonucleotides disclosed herein may be modified to facilitate targeting of a particular tissue, cell or organ, e.g., to facilitate delivery of the oligonucleotide to the CNS. In some embodiments, an oligonucleotide comprises a nucleotide that is conjugated to one or more targeting ligands.

A targeting ligand may comprise a carbohydrate, amino sugar, cholesterol, peptide, polypeptide, protein or part of a protein (e.g., an antibody or antibody fragment) or lipid. In some embodiments, a targeting ligand is an aptamer. For example, a targeting ligand may be an RGD peptide that is used to target tumor vasculature or glioma cells, CREKA peptide to target tumor vasculature or stoma, transferrin, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand, as described, for example, in International Patent Application Publication WO 2016/100401, the relevant contents of which are incorporated herein by reference.

In some embodiments, it is desirable to target an oligonucleotide that reduces the expression of ALDH2 to the cell of the CNS of a subject. GalNAc is a high affinity ligand for asialoglycoprotein receptor (ASGPR), which is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). In some embodiments, conjugation (either indirect or direct) of GalNAc moieties to oligonucleotides of the instant disclosure may be used to target these oligonucleotides to the ASGPR expressed on these hepatocyte cells. However, in some embodiments, GalNAc moieties may be used with oligonucleotides that are delivered directly to the CNS.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3, or 4 monovalent GalNAc moieties, and is typically conjugated to 3 or 4 monovalent GalNAc moieties). In some embodiments, an oligonucleotide of the instant disclosure is conjugated to one or more bivalent GalNAc, trivalent GalNAc, or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of the loop (L) of the stem-loop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a GalNAc moiety. In some embodiments, GalNAc moieties are conjugated to a nucleotide of the sense strand. For example, four GalNAc moieties can be conjugated to nucleotides in the tetraloop of the sense strand, where each GalNAc moiety is conjugated to one nucleotide.

In some embodiments, an oligonucleotide herein comprises a monovalent GalNAc attached to a Guanidine nucleotide, referred to as [ademG-GalNAc] or 2′-aminodiethoxymethanol-Guanidine-GalNAc, as depicted below:

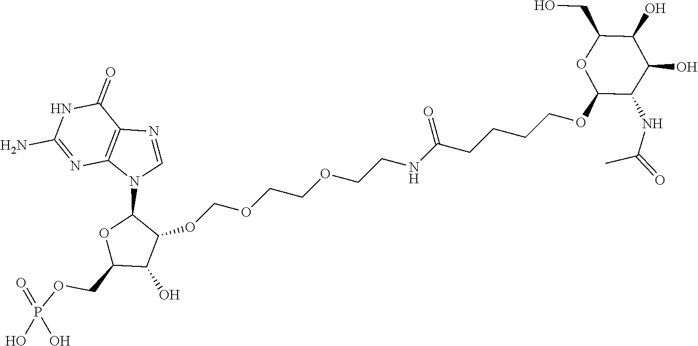 [SMILES: CC(=O)N[C@@H]1[C@@H](OCCCCC(=O)NCCOCCOCO[C@@H]2[C@H](O)[C@@H](COP(=O)(O)O)O[C@H]2n2cnc3c(=O)[nH]c(N)nc32)O[C@@H](CO)[C@@H](O)[C@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below.

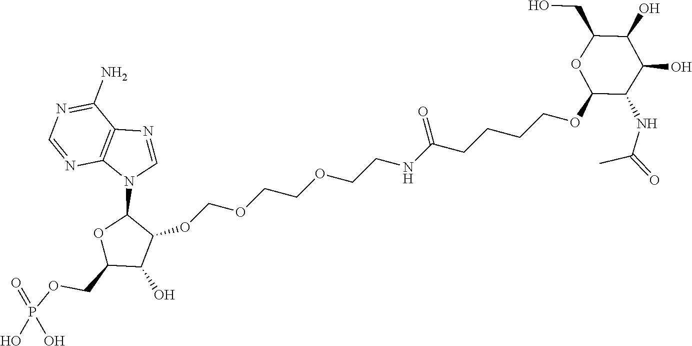

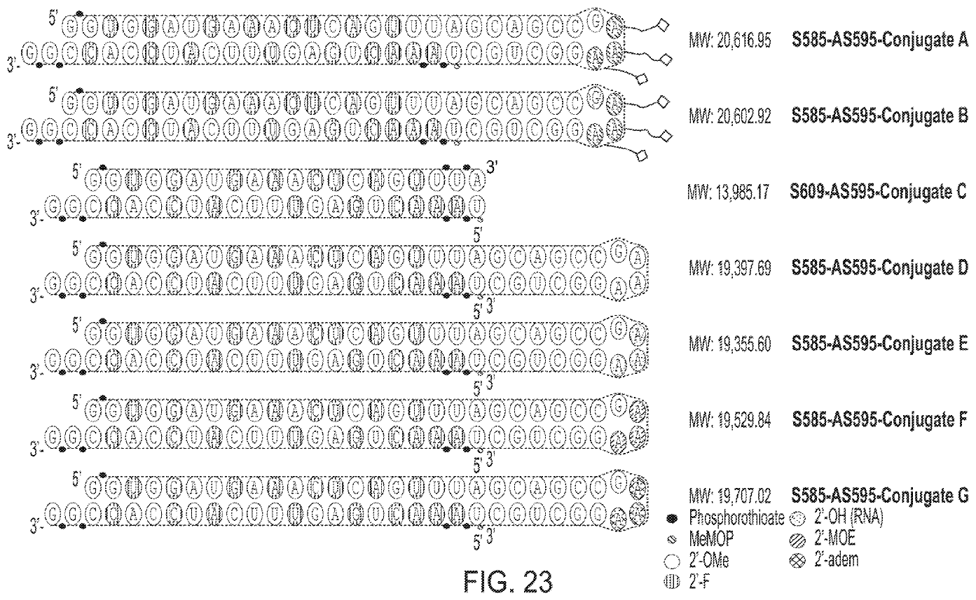

An example of such conjugation is shown below for a loop comprising from 5′ to 3′ the nucleotide sequence GAAA (L=linker, X=heteroatom) stem attachment points are shown. In some embodiments, such a loop may be present, for example, at positions 27-30 of sense strand oligonucleotides 36 nucleotides in length, such as presented in Appendix A and as illustrated in FIG. 23. In the chemical formula,

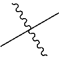

is used to describe an attachment point to the oligonucleotide strand.

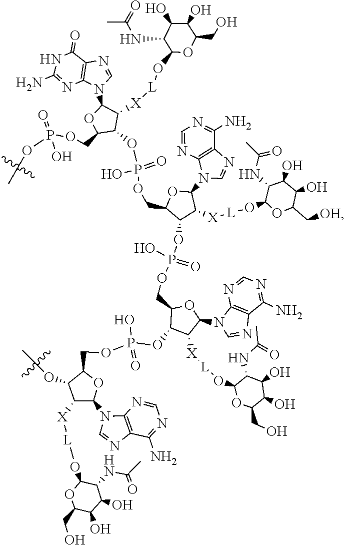

In some embodiments, L represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and X is O, S, or N. In some embodiments, L is an acetal linker. In some embodiments, X is O.

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in International patent publication WO2016100401, the contents of which relating to such linkers are incorporated herein by reference. In some embodiments, the linker is a labile linker. However, in other embodiments, the linker is stable. A “labile linker” refers to a linker that can be cleaved, e.g., by acidic pH. A “stable linker” refers to a linker that cannot be cleaved.

Another example is shown below for a loop comprising from 5′ to 3′ the nucleotides GAAA, in which GalNAc moieties are attached to nucleotides of the loop using an acetal linker. In some embodiments, such a loop may be present, for example, at positions 27-30 of sense strand oligonucleotides 36 nucleotides in length, such as presented in Appendix A, and as illustrated in FIG. 23. In the chemical formula,

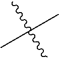

is an attachment point to the oligonucleotide strand.

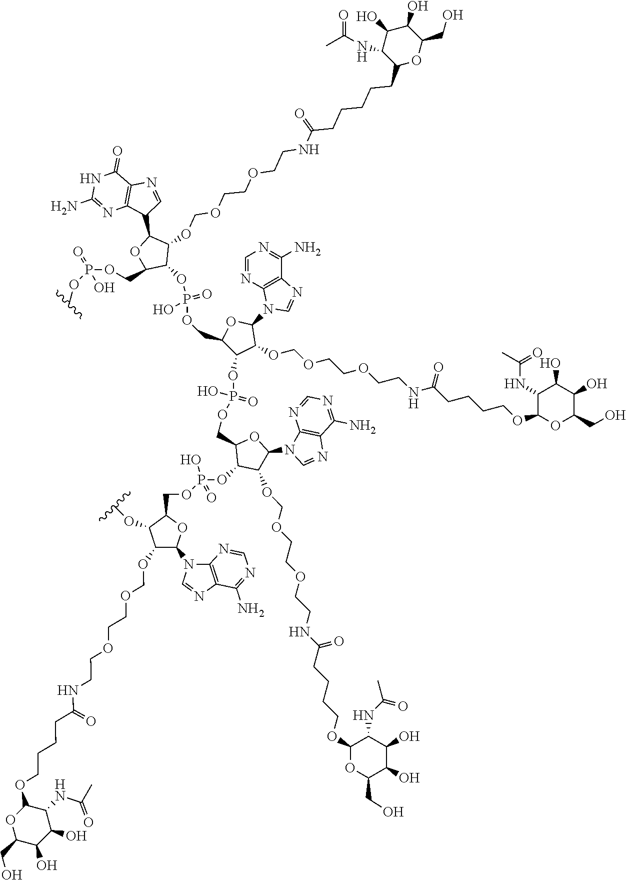

In some embodiments, the linker is a labile linker. However, in other embodiments, the linker is stable. In some embodiments, a duplex extension (up to 3, 4, 5, or 6 base pairs in length) is provided between a targeting ligand (e.g., a GalNAc moiety) and a double-stranded oligonucleotide.

In some embodiments, the GalNAc moiety is conjugated to each of A in the sequence GAAA, as illustrated in FIG. 23 for Conjugate A and Conjugate B. In some embodiments, the GalNAc moiety conjugated to each of A has the structure illustrated above, except that G is unmodified or has a 2′ modification on the sugar moiety. In some embodiments, the G in the GAAA sequence comprises a 2′ modification (e.g., 2′-O-methyl or 2′-O-methoxyethyl), and each of A in the GAAA sequence is conjugated to a GalNAc moiety, as illustrated in the structures above.

In some embodiments, the oligonucleotides of the present disclosure do not have a GalNAc conjugated. It was found herein that GalNAc conjugation is not required for neural cell uptake and oligonucleotide activity. In some embodiments, non-GalNAc-conjugated oligonucleotides have enhanced activity, compared to the GalNAc-conjugated counterparts.

vi. Oligonucleotide Derivatives

The present disclosure provides a range of oligonucleotide derivatives comprises a sense strand and an antisense strand, wherein the sense strand comprises a tetraloop comprising a L sequence set forth as GAAA, and wherein the sense strand and the antisense strand are not covalently linked. Different derivatives have different nucleotide modifications in the tetraloop.

In some embodiments, each of the A in GAAA sequence is conjugated to a GalNAc, and wherein the G in the GAAA sequence comprises a 2′-O-methyl modification. The oligonucleotide comprising this structure is termed herein as “Conjugate A.”

In some embodiments, each of the A in GAAA sequence and is conjugated to a GalNAc, and wherein the G in the GAAA sequence comprises a 2′-OH. The oligonucleotide comprising this structure is termed herein as “Conjugate B.”

In some embodiments, each of the nucleotides in the GAAA sequence is comprises a 2′-O-methyl modification. The oligonucleotide comprising this structure is termed herein as “Conjugate D.” Conjugate D does not have GalNAc conjugated to any of the nucleotides in the GAAA sequence.

In some embodiments, each of the A in the GAAA sequence comprises a 2′-OH and the G in the GAAA sequence comprises a 2′-O-methyl modification. The oligonucleotide comprising this structure is termed herein as “Conjugate E.” Conjugate E does not have GalNAc conjugated to any of the nucleotides in the GAAA sequence.

In some embodiments, each of the A in the GAAA sequence comprises a 2′-O-methoxyethyl (see, e.g., FIG. 23) modification and the G in the GAAA sequence comprises a 2′-O-methyl modification. The oligonucleotide comprising this structure is termed herein as “Conjugate F.” Conjugate F does not have GalNAc conjugated to any of the nucleotides in the GAAA sequence.

In some embodiments, each of the A in the GAAA sequence comprises a 2′-adem modification and the G in the GAAA sequence comprises a 2′-O-methyl modification. The oligonucleotide comprising this structure is termed herein as “Conjugate F.” Conjugate F does not have GalNAc conjugated to any of the nucleotides in the GAAA sequence.

In some embodiments, in any of the oligonucleotide derivatives described herein, the sense strand may comprise a sequence selected from SEQ ID NOs: 581-590 and the antisense strand may comprise a sequence selected from SEQ ID NOs: 591-600.

In some embodiments, the oligonucleotide derivative described herein comprises an antisense strand and a sense strand that are not covalently linked, wherein the antisense strand comprises a sequence as set forth in SEQ ID NO: 585 and the sense strand comprises a sequence as set forth in SEQ ID NO: 595, wherein the sense strand comprises at its 3′-end a stem-loop set forth as: S1-L-S2, wherein S1 is complementary to S2, and wherein L is a tetraloop comprising a sequence set forth as GAAA, and wherein the GAAA sequence comprises a structure selected from the group consisting of:

(i) each of the A in GAAA sequence is conjugated to a GalNAc moiety, and the G in the GAAA sequence comprises a 2′-O-methyl modification;

(ii) each of the A in GAAA sequence is conjugated to a GalNAc moiety, and the G in the GAAA sequence comprises a 2′-OH;

(iii) each of the nucleotide in the GAAA sequence comprises a 2′-O-methyl modification;

(iv) each of the A in the GAAA sequence comprises a 2′-OH and the G in the GAAA sequence comprises a 2′-O-methyl modification;

(v) each of the A in the GAAA sequence comprises a 2′-O-methoxyethyl modification and the G in the GAAA sequence comprises a 2′-O-methyl modification; and

(vi) each of the A in the GAAA sequence comprises a 2′-adem modification and the G in the GAAA sequence comprises a 2′-O-methyl modification.

In some embodiments, the oligonucleotide derivative described herein does not comprise a tetraloop in the sense strand (e.g., the 3′ end of the sense strand and the 5′ end of the antisense strand form a blunt end and the sense strand and the antisense strand are not covalently linked). The oligonucleotide comprising this structure is termed herein as “Conjugate F.” An exemplary Conjugate F may comprise a sense strand having the sequence set forth in SEQ ID NO: 609 and an antisense sequence having the sequence as set forth in SEQ ID NO: 595, where the antisense strand and the sense strand are not covalently linked.

In some embodiments, the oligonucleotide derivatives described herein further comprises different arrangements of 2′-fluoro and 2′-O-methyl modified nucleotides, phophorothioate linkages, and/or included a phosphate analog positioned at the 5′ terminal nucleotide of their antisense strands III. Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., single-stranded or double-stranded oligonucleotides) to reduce the expression of ALDH2. Such compositions can be suitably formulated such that when administered to a subject, either into the immediate environment of a target cell or systemically, a sufficient portion of the oligonucleotides enter the cell to reduce ALDH2 expression. Any of a variety of suitable oligonucleotide formulations can be used to deliver oligonucleotides for the reduction of ALDH2 as disclosed herein. In some embodiments, an oligonucleotide is formulated in buffer solutions such as phosphate-buffered saline solutions, liposomes, micellar structures, and capsids. In some embodiments, naked oligonucleotides or conjugates thereof are formulated in water or in an aqueous solution (e.g., water with pH adjustments). In some embodiments, naked oligonucleotides or conjugates thereof are formulated in basic buffered aqueous solutions (e.g., PBS).

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine) can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere, or a nanoparticle, or may be otherwise formulated for administration to the cells, tissues, organs, or body of a subject in need thereof (see, e.g., Remington: The Science and Practice of Pharmacy, 22nd edition, Pharmaceutical Press, 2013).

In some embodiments, the oligonucleotides are formulated with a pharmaceutically acceptable carrier, including excipients. In some embodiments, formulations as disclosed herein comprise an excipient or carrier. In some embodiments, an excipient or carrier confers to a composition improved stability, improved absorption, improved solubility and/or therapeutic enhancement of the active ingredient. In some embodiments, an excipient or carrier is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. The oligonucleotides of the present disclosure are administered to the cerebrospinal fluid of the subject. Suitable routes of administration include, without limitation, intraventricular, intracavitary, intrathecal, or interstitial administration.

Pharmaceutical compositions suitable for injectable use include sterile aqueous solutions (where water soluble) or dispersions and sterile powders for the extemporaneous preparation of sterile injectable solutions or dispersions. For intravenous or subcutaneous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. In many cases, it will be preferable to include isotonic agents, for example, sugars, polyalcohols such as mannitol, sorbitol, and sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., an oligonucleotide for reducing ALDH2 expression) or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### IV. Methods of Use

i. Reducing ALDH2 Expression in Cells

In some embodiments, methods are provided for delivering to a cell an effective amount any one of oligonucleotides disclosed herein for purposes of reducing expression of ALDH2 in the cell. Methods provided herein are useful in any appropriate cell type. In some embodiments, a cell is any cell that expresses ALDH2 (e.g., hepatocytes, macrophages, monocyte-derived cells, prostate cancer cells, cells of the central nervous system (e.g., neurons or glial cells), endocrine tissue, bone marrow, lymph nodes, lung, gall bladder, liver, duodenum, small intestine, pancreas, kidney, gastrointestinal tract, bladder, adipose and soft tissue and skin). In some embodiments, the cell is a primary cell that has been obtained from a subject and that may have undergone a limited number of a passages, such that the cell substantially maintains its natural phenotypic properties. In some embodiments, a cell to which the oligonucleotide is delivered is ex vivo or in vitro (i.e., can be delivered to a cell in culture or to an organism in which the cell resides). In specific embodiments, methods are provided for delivering to a cell an effective amount any one of the oligonucleotides disclosed herein for purposes of reducing expression of ALDH2 solely in the central nervous system (CNS).

In some embodiments, oligonucleotides disclosed herein can be introduced using appropriate nucleic acid delivery methods including injection of a solution containing the oligonucleotides, bombardment by particles covered by the oligonucleotides, exposing the cell or organism to a solution containing the oligonucleotides, or electroporation of cell membranes in the presence of the oligonucleotides. Other appropriate methods for delivering oligonucleotides to cells may be used, such as lipid-mediated carrier transport, chemical-mediated transport, and cationic liposome transfection such as calcium phosphate, and others.

The consequences of inhibition can be confirmed by an appropriate assay to evaluate one or more properties of a cell or subject, or by biochemical techniques that evaluate molecules indicative of ALDH2 expression (e.g., RNA, protein). In some embodiments, the extent to which an oligonucleotide provided herein reduces levels of expression of ALDH2 is evaluated by comparing expression levels (e.g., mRNA or protein levels of ALDH2 to an appropriate control (e.g., a level of ALDH2 expression in a cell or population of cells to which an oligonucleotide has not been delivered or to which a negative control has been delivered). In some embodiments, an appropriate control level of ALDH2 expression may be a predetermined level or value, such that a control level need not be measured every time. The predetermined level or value can take a variety of forms. In some embodiments, a predetermined level or value can be single cut-off value, such as a median or mean.

In some embodiments, administration of an oligonucleotide as described herein results in a reduction in the level of ALDH2 expression in a cell. In some embodiments, the reduction in levels of ALDH2 expression may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of ALDH2. The appropriate control level may be a level of ALDH2 expression in a cell or population of cells that has not been contacted with an oligonucleotide as described herein. In some embodiments, the effect of delivery of an oligonucleotide to a cell according to a method disclosed herein is assessed after a finite period. For example, levels of ALDH2 may be analyzed in a cell at least 8 hours, 12 hours, 18 hours, 24 hours; or at least one, two, three, four, five, six, seven, or fourteen days after introduction of the oligonucleotide into the cell.

In some embodiments, an oligonucleotide is delivered in the form of a transgene that is engineered to express in a cell the oligonucleotides (e.g., its sense and antisense strands). In some embodiments, an oligonucleotide is delivered using a transgene that is engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes can be injected directly to a subject.

ii. Treatment Methods

In another aspect, the present disclosure relates to methods for reducing ALDH2 expression for the treatment of a neurological disease in a subject. In some embodiments, the methods may comprise administering to the cerebrospinal fluid of a subject in need thereof an effective amount of any one of the oligonucleotides disclosed herein. Such treatments could be used, for example, to reduce ALDH2 expression in the central nervous system (e.g., somatosensory cortex, hippocampus, frontal cortex, striatum, hypothalamus, cerebellum, and across the spinal cord). The present disclosure provides for both prophylactic and therapeutic methods of treating a subject at risk of (or susceptible to) a neurological disease. In some embodiments, the present disclosure provides methods or use of the oligonucleotides for treating a neurological disorder. In some embodiments, the neurological disorder is a neurodegenerative disease, cognitive disorder, or anxiety disorder. Exemplary neurological disorders associated with ALDH2 expression in the CNS include, among others, senile dementia, dyskinesia, Alzheimer's disease (AD), and Parkinson's disease (PD).

In certain aspects, the disclosure provides a method for preventing in a subject, a disease or disorder as described herein by administering to the subject a therapeutic agent (e.g., an oligonucleotide or vector or transgene encoding same). In some embodiments, the subject to be treated is a subject who will benefit therapeutically from a reduction in the amount of ALDH2 protein, e.g., in the central nervous system.

Methods described herein typically involve administering to a subject an effective amount of an oligonucleotide, that is, an amount capable of producing a desirable therapeutic result. A therapeutically acceptable amount may be an amount that is capable of treating a disease or disorder. The appropriate dosage for any one subject will depend on certain factors, including the subject's size, body surface area, age, the composition to be administered, the active ingredient(s) in the composition, time and route of administration, general health, and other drugs being administered concurrently.

In some embodiments, a subject is administered any one of the compositions disclosed herein to the cerebrospinal fluid (CSF) of a subject, e.g., by injection or infusion. In some embodiments, oligonucleotides disclosed herein are delivered via intraventricular, intracavitary, intrathecal, or interstitial administration.

In some embodiments, oligonucleotides are administered at a dose in a range of 0.1 mg/kg to 25 mg/kg (e.g., 1 mg/kg to 5 mg/kg). In some embodiments, oligonucleotides are administered at a dose in a range of 0.1 mg/kg to 5 mg/kg or in a range of 0.5 mg/kg to 5 mg/kg.

As a non-limiting set of examples, the oligonucleotides of the instant disclosure would typically be administered once per year, twice per year, quarterly (once every three months), bi-monthly (once every two months), monthly, or weekly.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

iii. Reducing Target Gene Expression in Cells

In some aspects the present disclosure provides methods of using the oligonucleotide derivatives (e.g., Conjugates A, B, C, D, E, F, or G) for reducing the expression of a target gene in a subject.

In some embodiments, the method comprises administering any of the oligonucleotide derivatives (e.g., Conjugates A, B, C, D, E, F, or G) to the cerebrospinal fluid of the subject. The antisense and sense strand of the oligonucleotide can be engineered to target any target gene. In some embodiments, the antisense strand is 21 to 27 nucleotides in length and has a region of complementarity to the target gene.

Other genes that could be targeted with the methods and oligonucleotides described herein include those identified as causing: Spinocerebellar Ataxia Type 1 (Ataxin-1, and/or Ataxin-3); the β-amyloid precursor protein gene (APP or BACE1) or mutants thereof; Dystonia (DYT1); Amyotrophic Lateral Sclerosis “ALS” or Lou Gehrig's Disease (SOD1) and, various genes that lead to tumors in the CNS.

In some embodiments, the gene of interest is selected from the group consisting of ALDH2, Ataxin-1, Ataxin-3, APP, BACE1, DYT1, and SOD1.

### EXAMPLES

### Example 1: Delivery of GalNAc-Conjugated ALDH2 Oligonucleotide to the Central Nervous System (CNS)

The central nervous system (CNS) is a protected environment. The circulating protein content in the cerebrospinal fluid (CSF) is less than 1% of that in plasma, and the CSF has little intrinsic nuclease activity. The CNS is ‘immune-privileged’ because the blood-brain barrier prevents circulation of immune cells. Oligonucleotides administered into CSF distribute via CSF bulk flow and have extended tissue half-lives (up to 200 days in brain and spinal cord following intracerebroventricular (ICV) infusion). Neural cells readily take up oligonucleotides. The size and/or lipophilicity of RNAi oligonucleotides can be engineered to reduce their elimination from CSF. However, RNAi oligonucleotides do not cross the blood-brain barrier, and thus require direct administration into the CNS (e.g., intrathecal or ICV injection). Oligonucleotides are cleared from CSF via lymphatic system and subject to same considerations/limitations as systemically administered oligonucleotides (e.g., renal toxicity, thrombocytopenia). In one embodiment of the present disclosure, the active guide strands are prepared in larger oligonucleotide carriers that are chemically modified to protect the compound against rapid elimination from the CNS. The chemical modification to the oligonucleotide carrier includes simply larger molecular size, lipophilicity, dimerization, modifications to charge or polarity, increase in molecular weight each in an effort to reduce or slow the ability of the CNS to remove the overall molecule until the guide strand can load into the RISC and inhibit the target mRNA.

In some embodiments, when eliminated from the CNS and located in another bodily compartment the oligonucleotides of the current invention are modified to be easily accessible to nucleases and other degradative molecules such that oligonucleotides outside the CNS are easily degraded. In this way off target effects are limited or prevented.

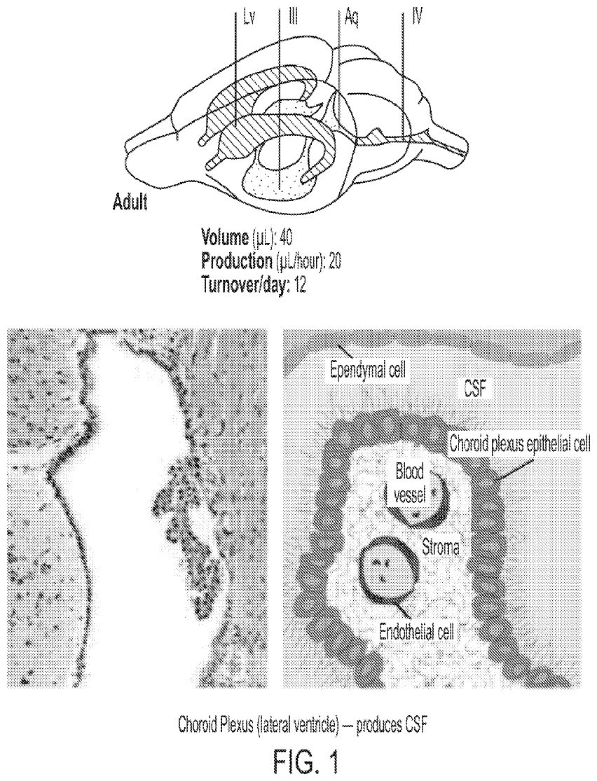

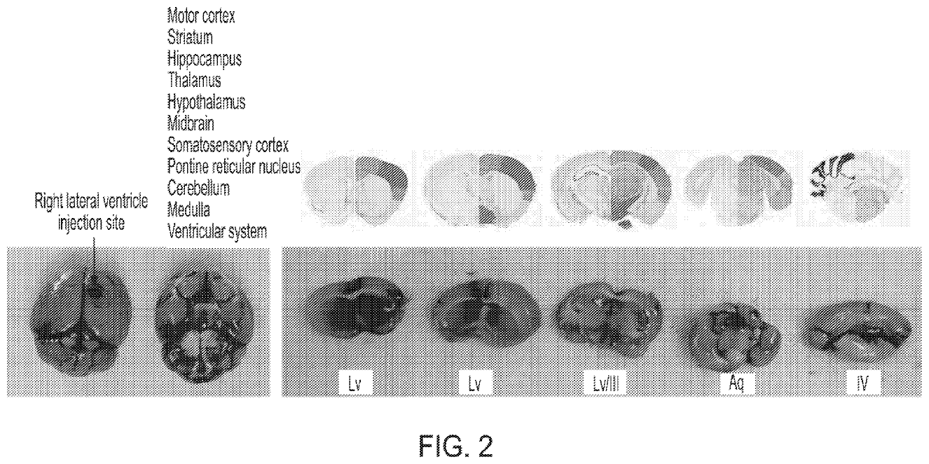

In this study, GalNAc-conjugated ALDH2 oligonucleotides were delivered to the CNS of female CD-1 mice via direct intraventricular injection (FIG. 1). It was first shown that FastGreem dye injected to the right lateral ventricle injection site distributed throughout the ventricular system (FIG. 2).

GalNAc-conjugated ALDH2 oligonucleotides are effective in reducing ALDH2 expression in the liver but is rapidly cleared from CNS compartment. Two derivatives of the S585-AS595-Conjugate A oligonucleotide (S608-AS595-Conjugate A and S608-AS595-Conjugate A-PS tail) were designed to enhance CSF retention. These oligonucleotides further comprise a combination of 2′-fluoro and 2′-O-methyl modified nucleotides, phophorothioate linkages, and/or include a phosphate analog positioned at the 5′ terminal nucleotide of their antisense strands.

The phosphothioate (PS)-modified nucleotides at the 3′ portion of the antisense strand was predicted to enhance CSF retention and neural cell uptake. A non-PS-modified tail included as control to decouple the contributions of PS modifications or asymmetry in mediating uptake.

To study the activities of the GalNAc-conjugated ALDH2 oligonucleotides (parent and derivatives) in reducing ALDH2 expression in the central nervous system, the GalNAc-conjugated ALDH2 oligonucleotides (parent and derivatives) were administered to mice (n=4 for each group) via direct intraventricular injection (ICV) and the remaining ALDH2 mRNA level in different regions of the mice brain were assessed 5 days post administration. The study design is shown in Table 1.

TABLE 1
CNS activity study design

| | | | | Stock solution | |
|---|---|---|---|---|---|
| Group | Route | *Dose (μg) | Volume (μl) | (mg/ml) | Oligonucleotide |
| A | ICV | NA | 10 | 10 | NA |
| B | ICV | 100 | 10 | 10 | S585-AS595- |
| | | | | | Conjugate A |
| C | ICV | 100 | 10 | 10 | S608-AS595- |
| | | | | | Conjugate A |
| D | ICV | 100 | 10 | 10 | S608-AS595- |
| | | | | | Conjugate A-PS |

*100 μg does is equivalent to 4 mg/kg.

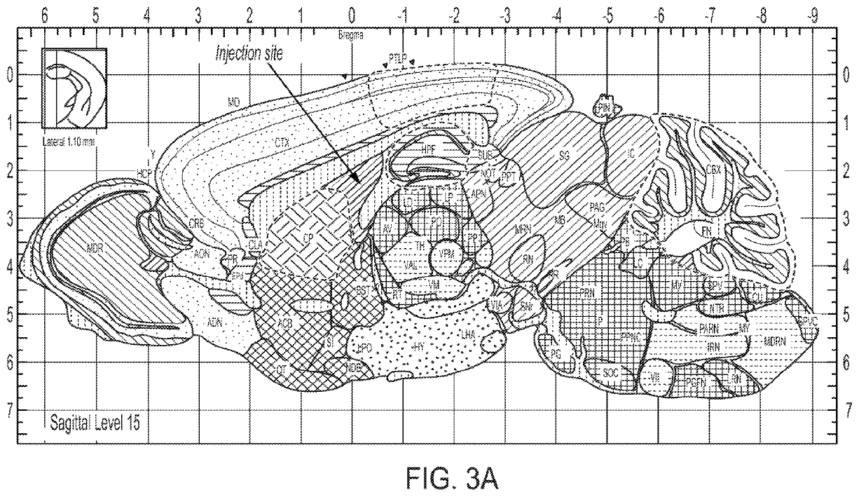

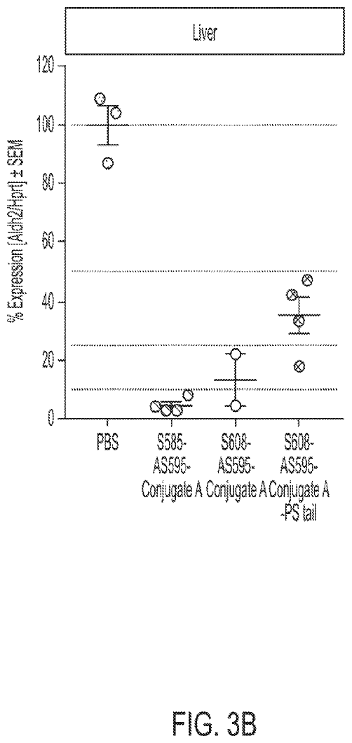

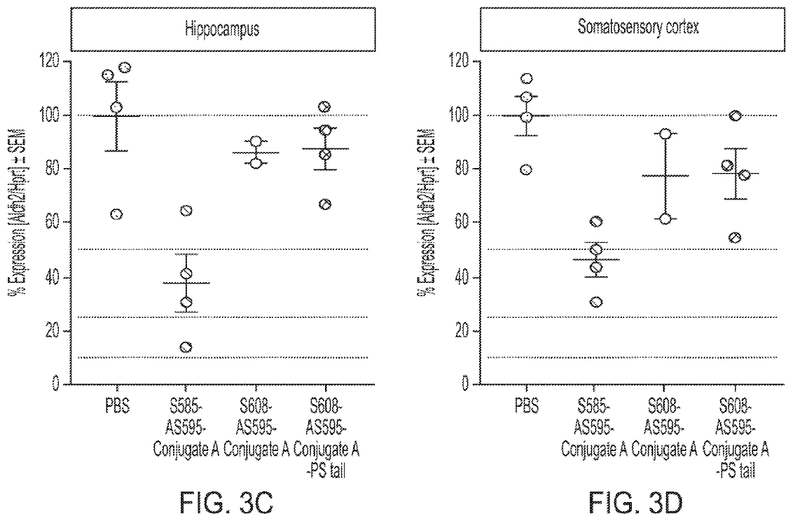

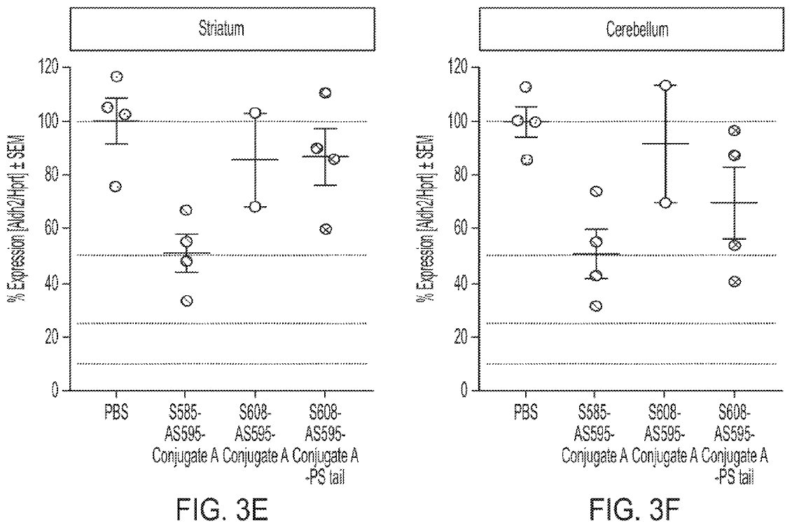

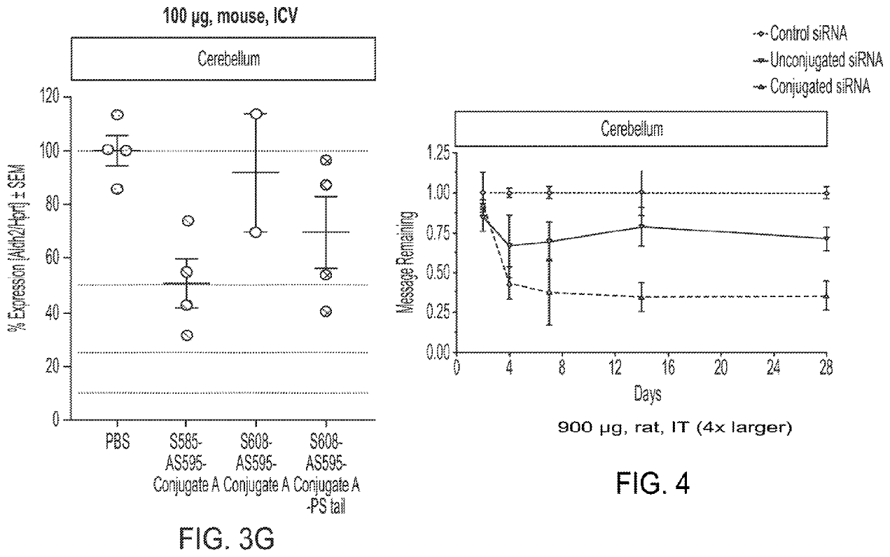

The result shows that all tested GalNAc-conjugated ALDH2 oligonucleotides reduced ALDH2 expression in different brain regions and in the liver (FIG. 3). Further, as demonstrated in FIG. 4, one single 100 μg does of GalNAc-conjugated ALDH2 oligonucleotides administered to mice via ICV administration showed similar activities in reducing ALDH2 expression in the cerebellum, compared to a benchmark 900 μg dose (in rat) via intrathecal administration for a different RNAi oligonucleotide (conjugated or unconjugated).

### Example 2. Dose Response of GalNAc-Conjugated ALDH2 Oligonucleotides in the CNS

The GalNAc-conjugated ALDH2 oligonucleotide (S585-AS595-Conjugate A) was tested using the same assay as above, but at two different concentrations (250 μg and 500 μg). The GalNAc-conjugated ALDH2 oligonucleotide was administered to mice via ICV and tissues (Striatum, cortex (somatosensory and frontal), hippocampus, hypothalamus, cerebellum, spinal cord) were collected at day 7 or day 28 post administration. The remaining ALDH2 mRNA level in the tissues were assessed using RT-PCT. The amount of the GalNAc-conjugated ALDH2 oligonucleotide in the tissues were assessed using SL-qPCT. The study design is shown in Table 2.

TABLE 2
Dose response study design

| Group | Route | *Dose (μg) | Volume (μl) | Stock solution (mg/ml) |
|---|---|---|---|---|
| A | ICV | NA | 10 | NA |
| B | ICV | 250 | 10 | 25 |
| C | ICV | 500 | 10 | 50 |
| D | ICV | 250 | 10 | 25 |
| E | ICV | 500 | 10 | 50 |

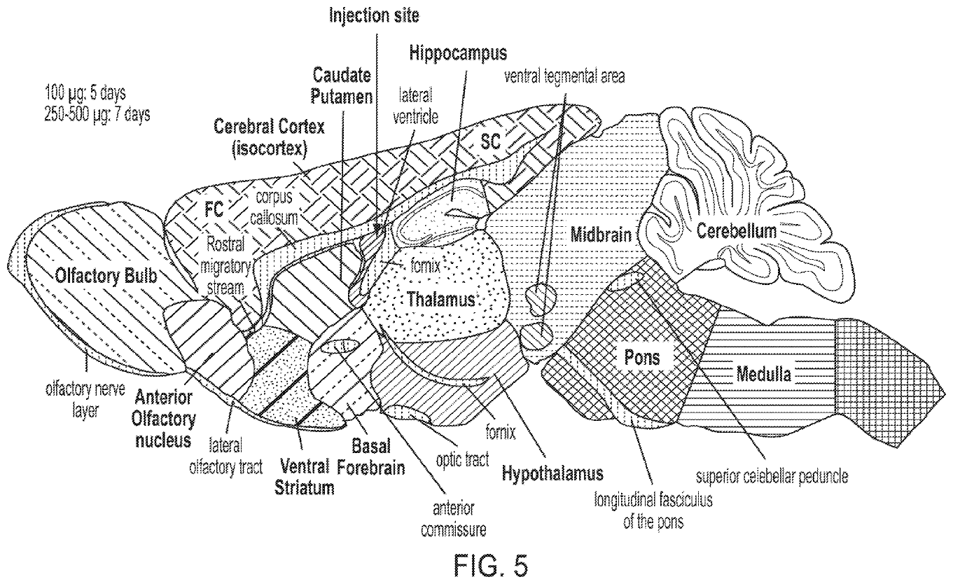

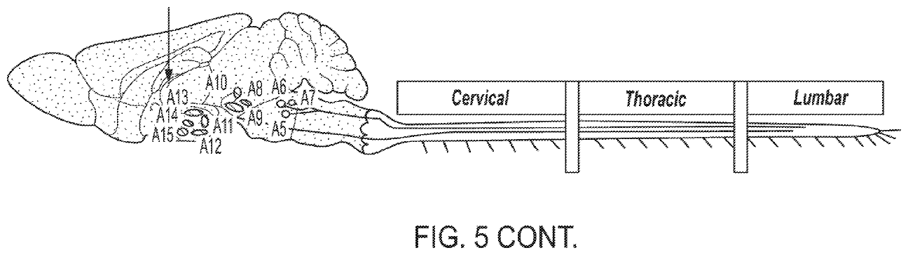

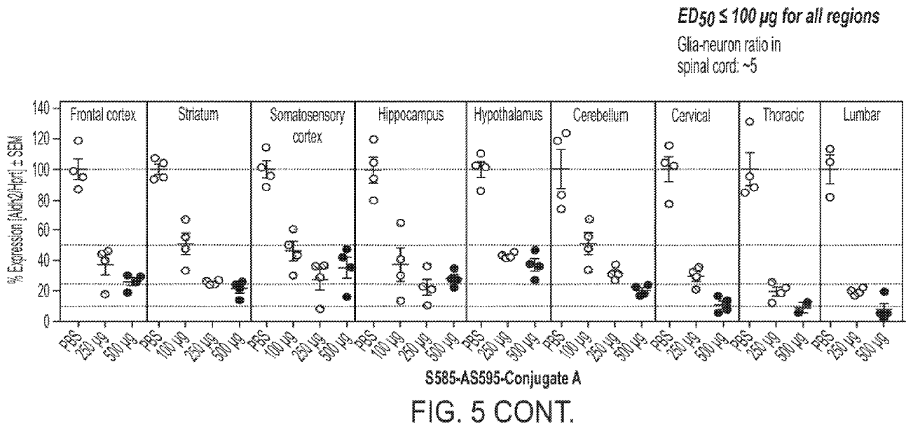

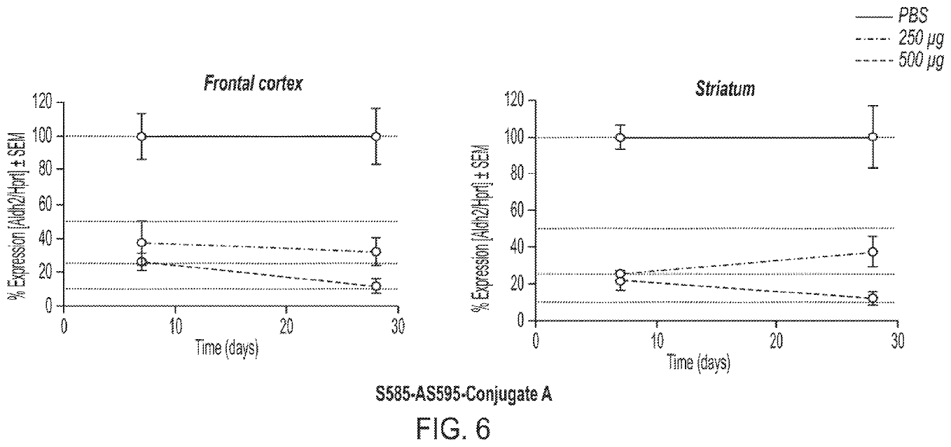

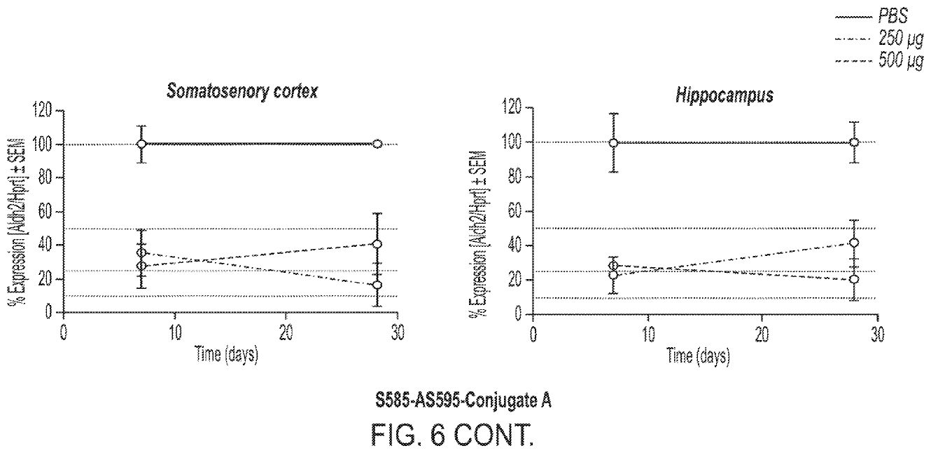

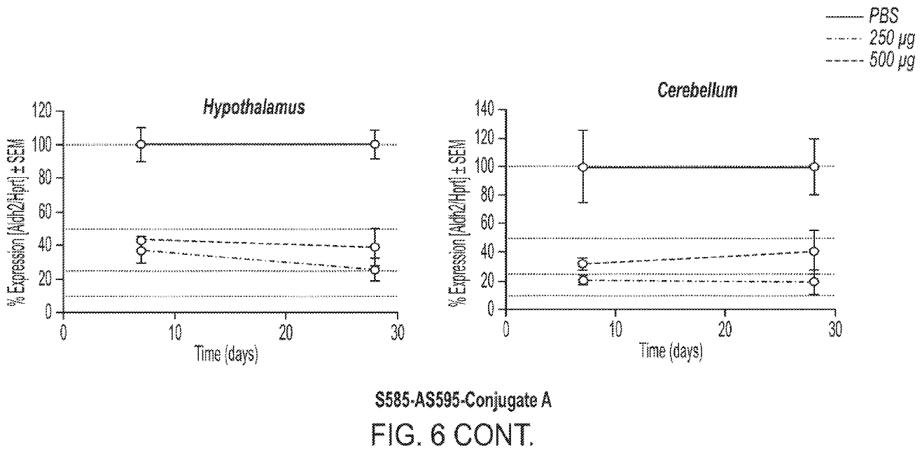

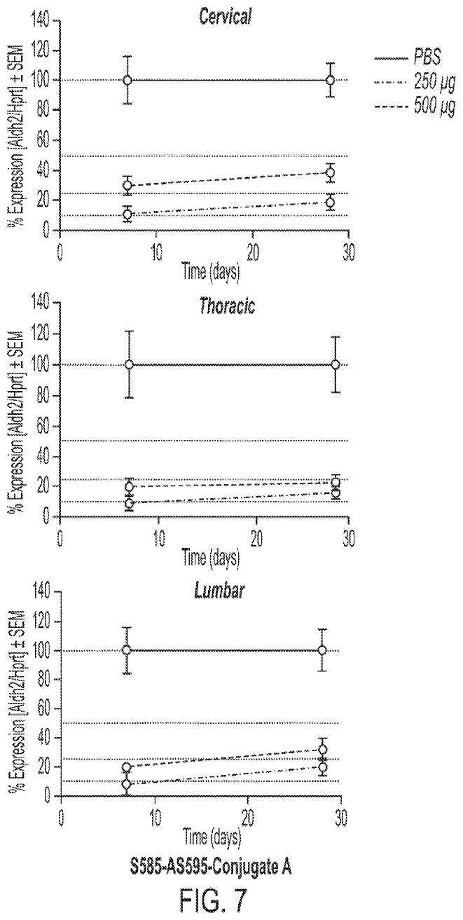

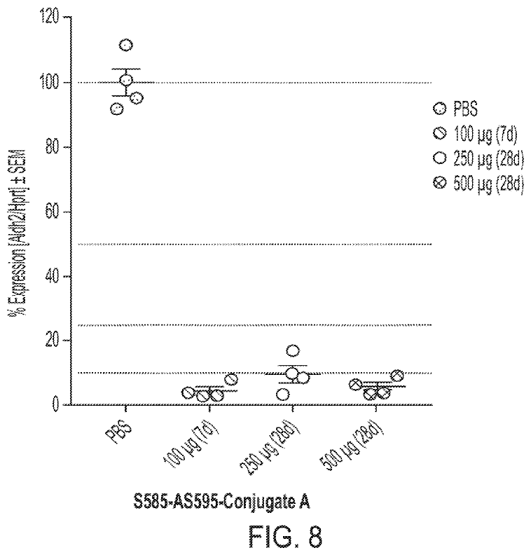

The results show that the GalNAc-conjugated ALDH2 oligonucleotide (5585-AS595-Conjugate A) significantly reduced ALDH2 mRNA level in all brain and spinal cord regions 7 days post administration (FIG. 5). E_{D}50 is less than 100 μg for all regions. Note in FIG. 7, results for 100 μg dose obtained on day 5 were also included. Sustained silencing of ALDH2 mRNA expression was also observed throughout the brain (FIG. 6) and across the spinal cord (FIG. 7) over 28 days following a single, ICV injection of the GalNAc-conjugated ALDH2 oligonucleotide at 250 μg or 500 μg doses. The ICV injected the GalNAc-conjugated ALDH2 oligonucleotide also reduced ALDH2 expression level in the level 7 and 28 days after administration (FIG. 8).

### Example 3. CNS Duration of the Effect of GalNAc-Conjugated ALDH2 Oligonucleotide

The duration of effect of GalNAc-conjugated ALDH2 oligonucleotide (5585-AS595-Conjugate A) in the brain and spinal cord after a single, bolus ICV injection was also assessed. GalNAc-conjugated ALDH2 oligonucleotide were to CD-1 female mice (6-8 weeks of age) delivered via ICV injection to the right lateral ventricle at two dose levels, 250 μg and 500 μg. Mice were sacrificed 7, 28, and 56 days after infusion and tissues (Striatum, cortex (somatosensory and frontal), hippocampus, hypothalamus, spinal cord) were collected. The remaining ALDH2 mRNA level in the tissues were assessed using RT-PCT. The study design is shown in Table 3 below.

TABLE 3
Duration study

| Group | Route | *Dose (μg) | Volume (μl) | Stock solution (mg/ml) |
|---|---|---|---|---|
| A | ICV | NA | 10 | NA |
| B | ICV | 250 | 10 | 25 |
| C | ICV | 500 | 10 | 50 |

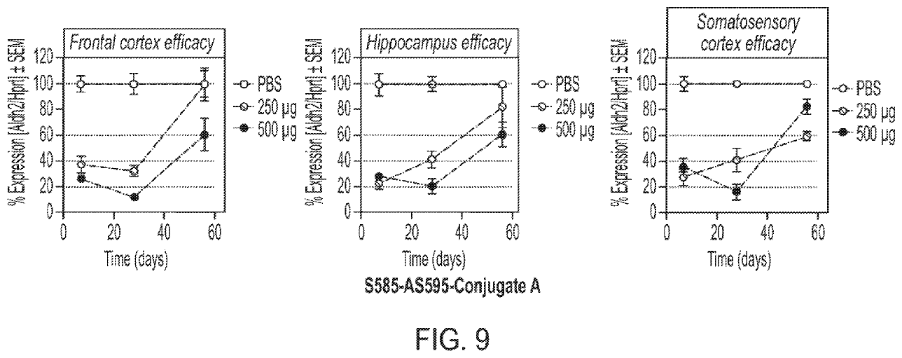

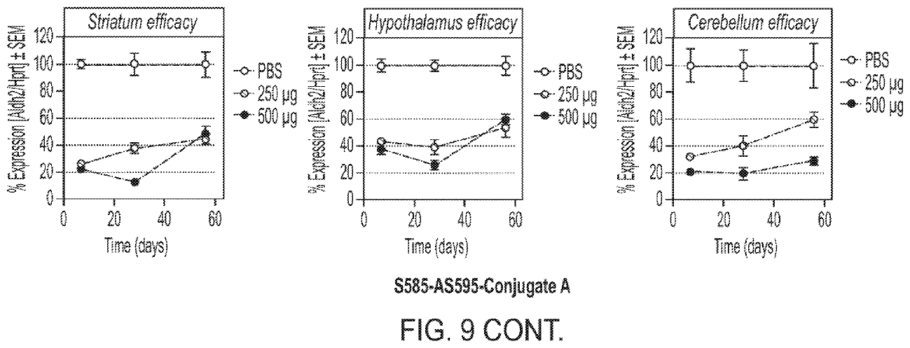

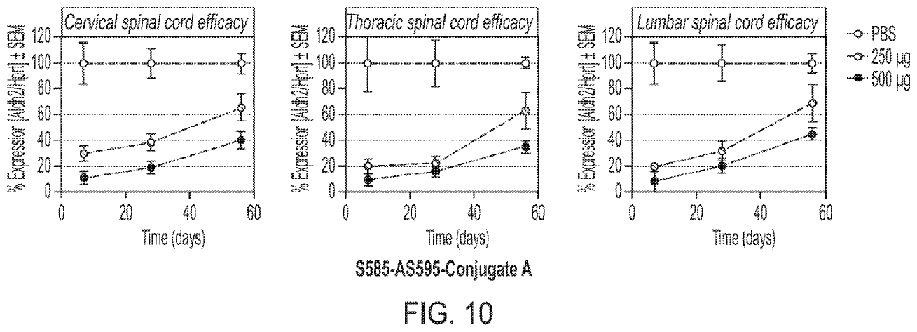

The results show that the ALDH2 reducing effect of the GalNAc-conjugated ALDH2 oligonucleotide (S585-AS595-Conjugate A) lasted around 30 days in different regions of the brain (FIG. 9) and across the spinal cord (FIG. 10). After 30 days, the remaining ALDH2 mRNA level increased overtime, but did not rise to the mRNA level before knockdown in at the 56-day time point.

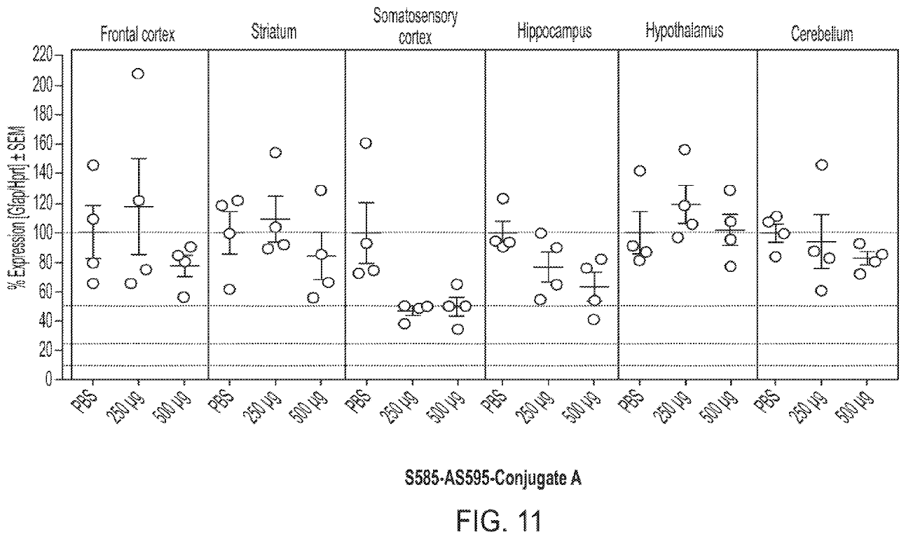

The neurotoxicity of the GalNAc-conjugated ALDH2 oligonucleotide (S585-AS595-Conjugate A) was also assessed. No Gfap upregulation was observed following administration of either 250 μg or 500 μg of the GalNAc-conjugated ALDH2 oligonucleotide (FIG. 11). No gliosis (reactive change in glial cells in response to CNS injury) was observed indicating tolerability. Toxicity and therapeutic efficacy of those compounds described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds which exhibit high therapeutic indices on this scale are preferred. While compounds that exhibit toxic side effects may be used, care should be taken to design a delivery system that targets such compounds to the site of affected tissue in order to minimize potential damage to uninfected cells and, thereby, reduce side effects.

### Example 4. ALDH2 RNAi Oligonucleotide Derivatives

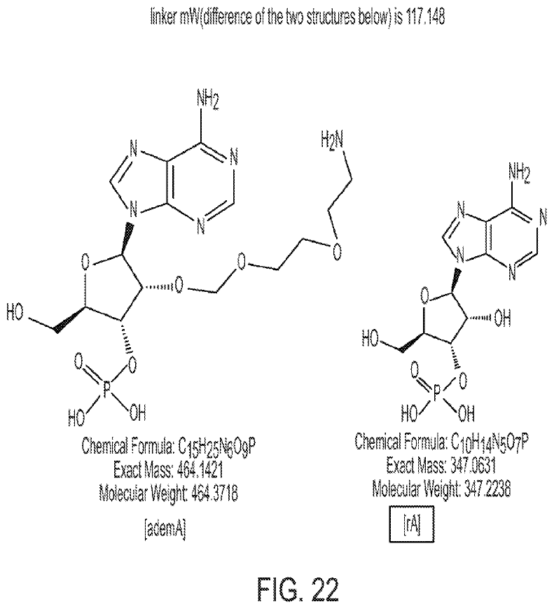

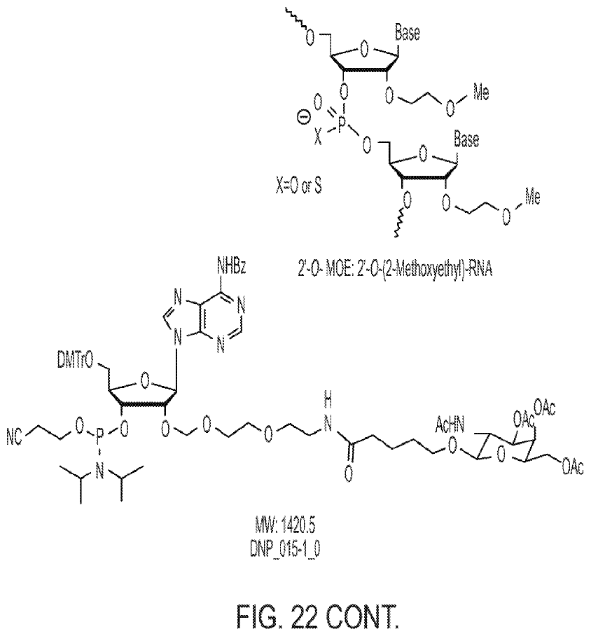

To determine whether GalNAc conjugation is required for neuronal delivery and to identify of structural variants of the GalNAc-conjugated ALDH2 oligonucleotide that have ALDH2 inhibiting activity in the CNS, a panel of ALDH2 RNAi oligonucleotide derivatives were designed (Conjugates A-G, FIG. 23). All derivatives form different structures at the 5′ end of the sense strand, with or without a tetraloop structure. Exemplary modified nucleotides in the tetraloop portion of the oligonucleotide derivatives are shown in FIG. 22. Additionally, all further comprise a combination of 2′-fluoro and 2′-O-methyl modified nucleotides, phophorothioate linkages, and/or include a phosphate analog positioned at the 5′ terminal nucleotide of their antisense strands.

Conjugates A, B, D, E, F, and G comprise a tetraloop comprising a sequence set forth as GAAA and comprise a sense strand having a sequence as set forth in SEQ ID NO: 585, and an antisense strand having a sequence as set forth in SEQ ID NO: 595. Conjugate C does not contain a tetraloop and the 3′ of the sense strand and the 5′ end of the anti-sense strand form a blunt end. Conjugate C comprises a sense strand having a sequence as set forth in SEQ ID NO: 609, and an antisense strand having a sequence as set forth in SEQ ID NO: 595.

In Conjugate A, each of the A in GAAA sequence is conjugated to a GalNAc moiety and the G in the GAAA sequence comprises a 2′-O-methyl modification.

In Conjugate B, each of the A in GAAA sequence is conjugated to a GalNAc moiety and the G in the GAAA sequence comprises a 2′-OH.

In Conjugate D, each of the nucleotide in the GAAA sequence comprises a 2′-O-methyl modification.

In Conjugate E, each of the A in the GAAA sequence comprises a 2′-OH and the G in the GAAA sequence comprises a 2′-O-methyl modification.

In Conjugate F, each of the A in the GAAA sequence comprises a 2′-O-methoxyethyl modification and the G in the GAAA sequence comprises a 2′-O-methyl modification.

In Conjugate G, each of the A in the GAAA sequence comprises a 2′-adem and the G in the GAAA sequence comprises a 2′-O-methyl modification.

The activities of the derivatives in reducing ALDH2 expression in the CNS were assessed. A single, bolus ICV injection of the ALDH2 RNAi oligonucleotide derivatives to CD-1 female mice (6-8 weeks of age, n=4). The derivatives were delivered via ICV injection to the right lateral ventricle at 200 μg. Mice were sacrificed 14 days after infusion and tissues (Somatosensory cortex, hippocampus, striatum, frontal cortex, cerebellum, hypothalamus, cervical spinal cord, thoracic spinal cord, lumbar spinal cord, liver) were collected. The remaining ALDH2 mRNA level in the tissues were assessed using RT-PCT. The amount of the ALDH2 RNAi oligonucleotide derivatives in the tissues were assessed using SL-qPCT. The study design is shown in Table 4.

TABLE 4
Activities of ALDH2 RNAi oligonucleotide derivatives

| | | | | Stock solution | |
|---|---|---|---|---|---|
| Group | Route | *Dose (μg) | Volume (μl) | (mg/ml) | Oligonucleotide |
| A | ICV | NA | 10 | NA | NA |
| B | ICV | 200 | 10 | 20 | S585-AS595- |
| | | | | | Conjugate A |
| C | ICV | 200 | 10 | 20 | S585-AS595- |
| | | | | | Conjugate B |
| D | ICV | 200 | 10 | 20 | S609-AS595- |
| | | | | | Conjugate C |
| E | ICV | 200 | 10 | 20 | S585-AS595- |
| | | | | | Conjugate D |
| F | ICV | 200 | 10 | 20 | S585-AS595- |
| | | | | | Conjugate E |
| G | ICV | 200 | 10 | 20 | S585-AS595- |
| | | | | | Conjugate F |
| H | ICV | 200 | 10 | 20 | S585-AS595- |
| | | | | | Conjugate G |

*Systemic dose equivalency: ~ 8 mg/kg for tetraloop structures, ~13.5 mg/kg for shortened duplex

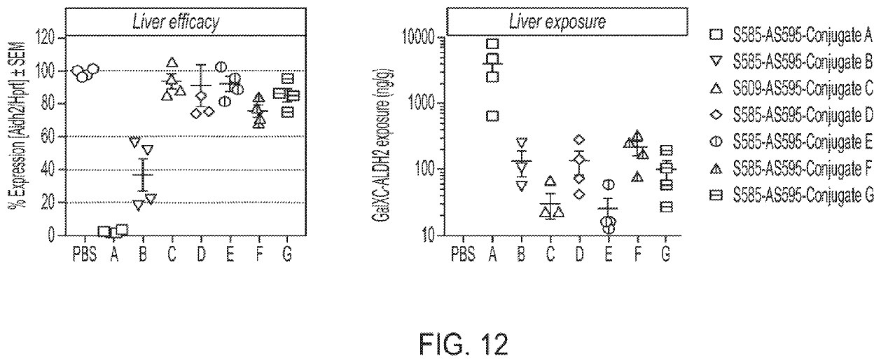

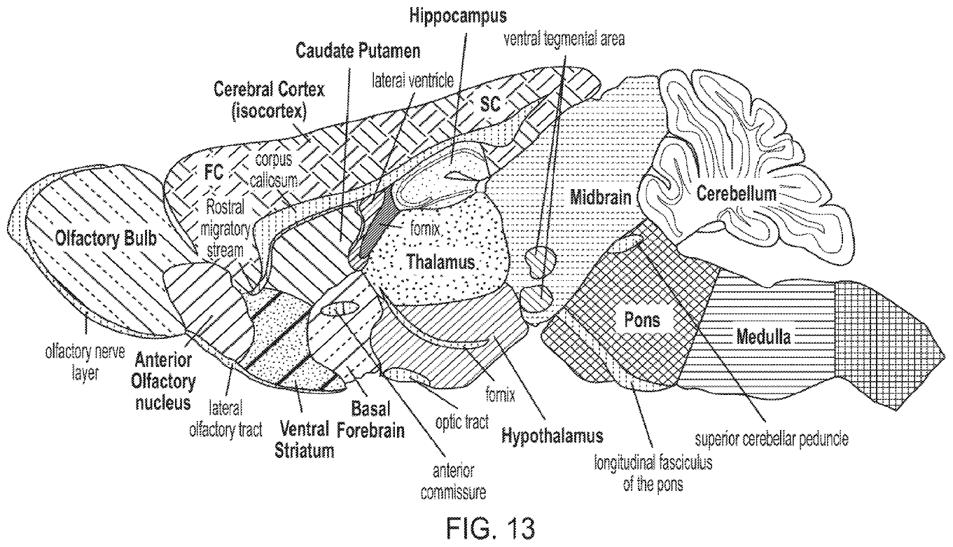

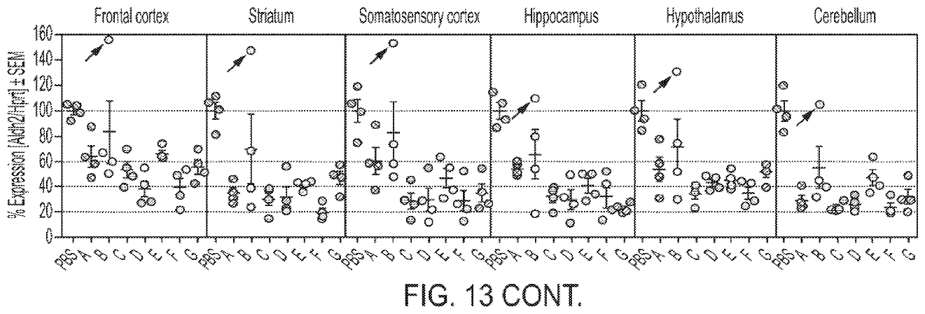

FIG. 12 shows that the non-GalNAc-conjugated oligonucleotides are inactive in the liver after two weeks. Conjugate B is still partially active in liver, likely due to high dose (8 mg/kg equivalent). FIG. 13 shows that GalNAc conjugation is not required for oligonucleotide efficacy throughout the brain.

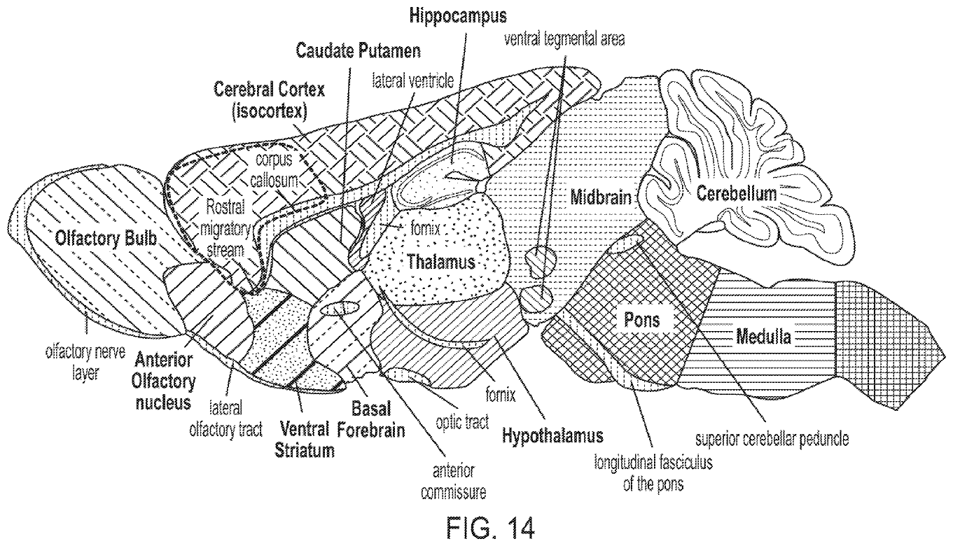

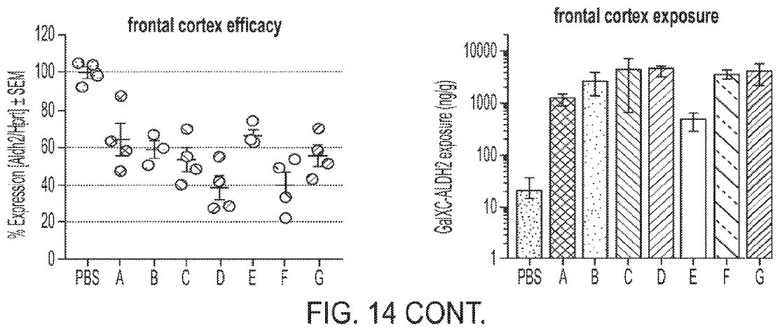

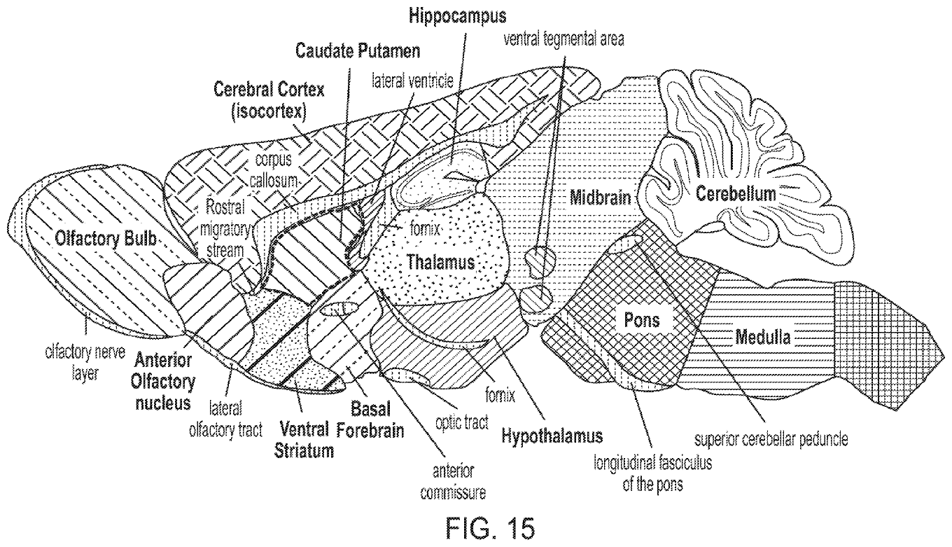

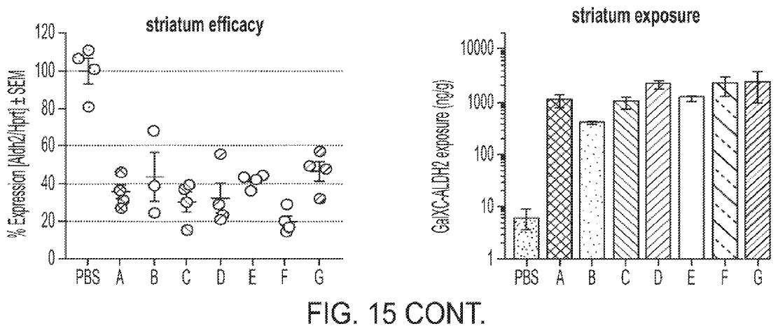

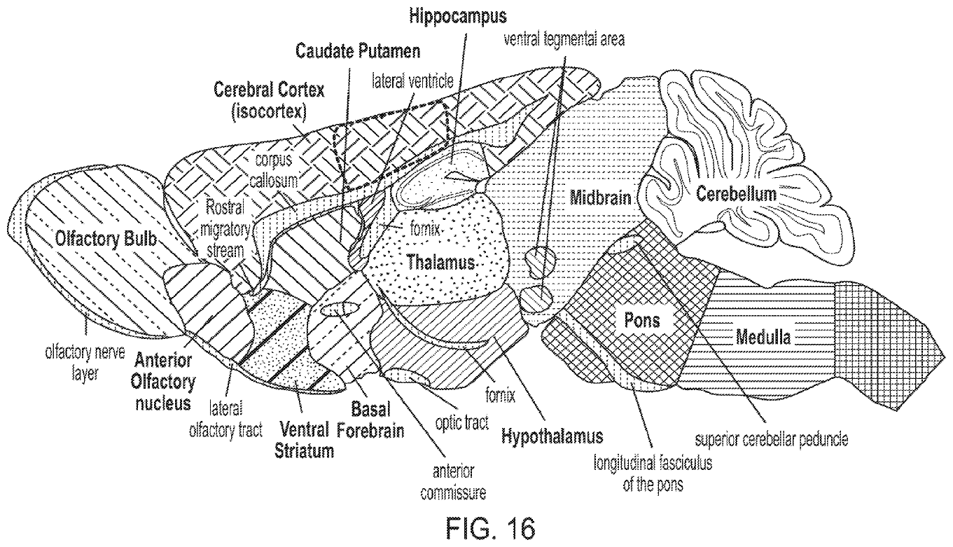

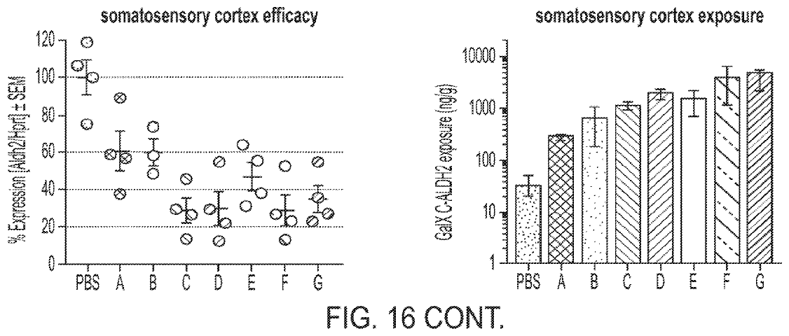

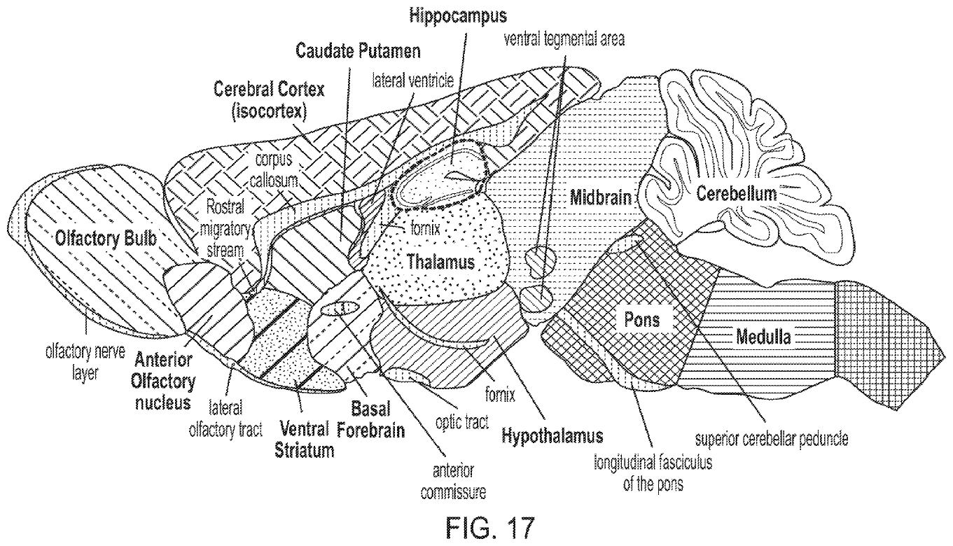

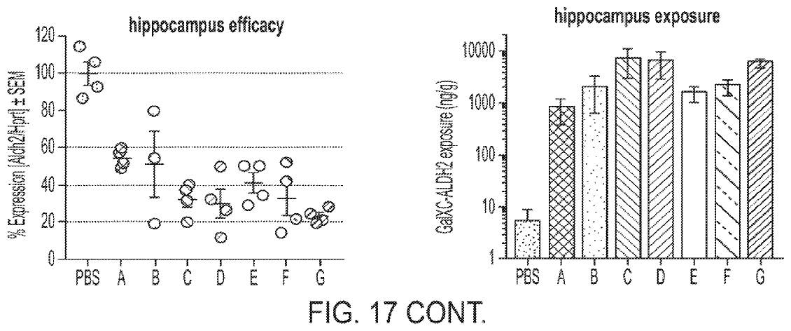

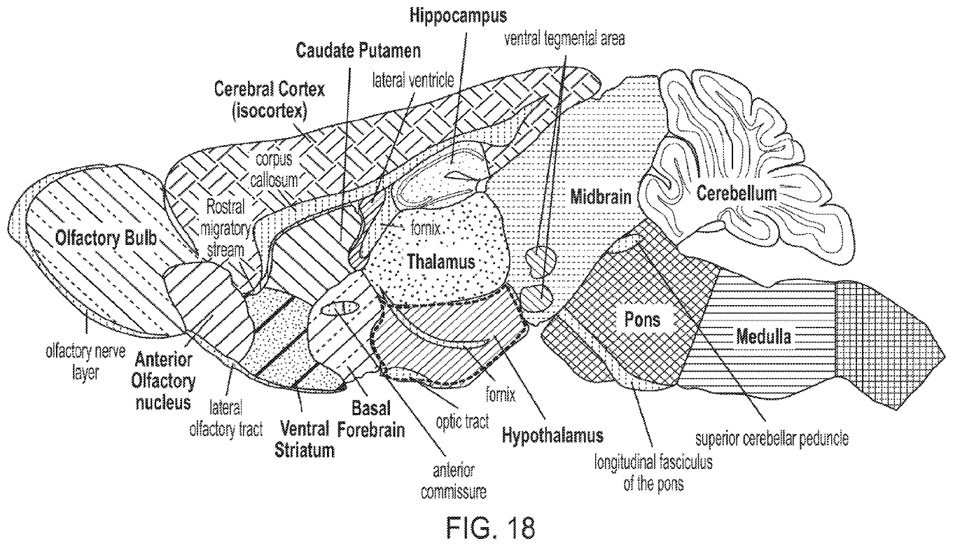

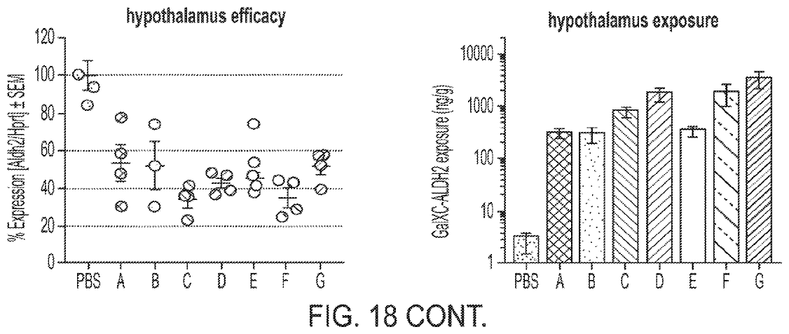

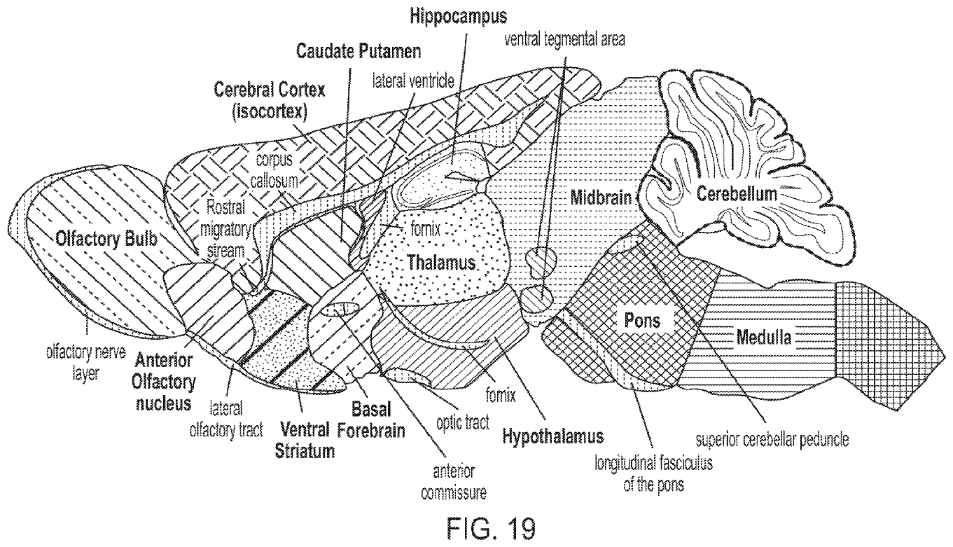

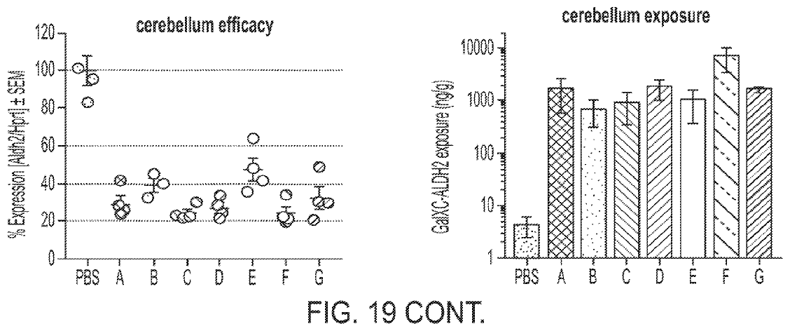

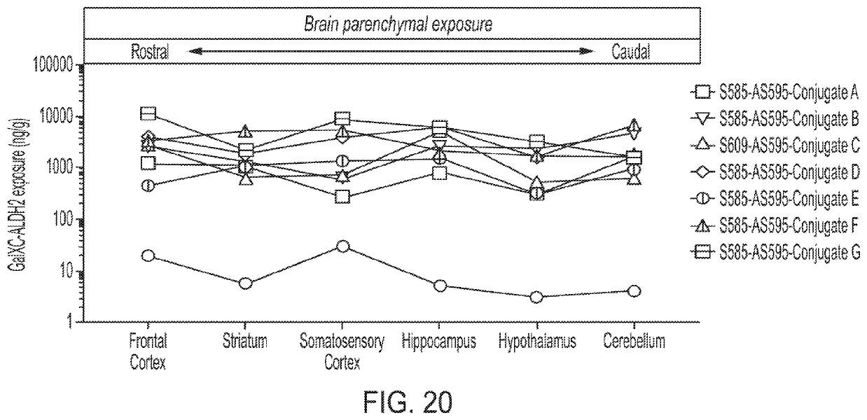

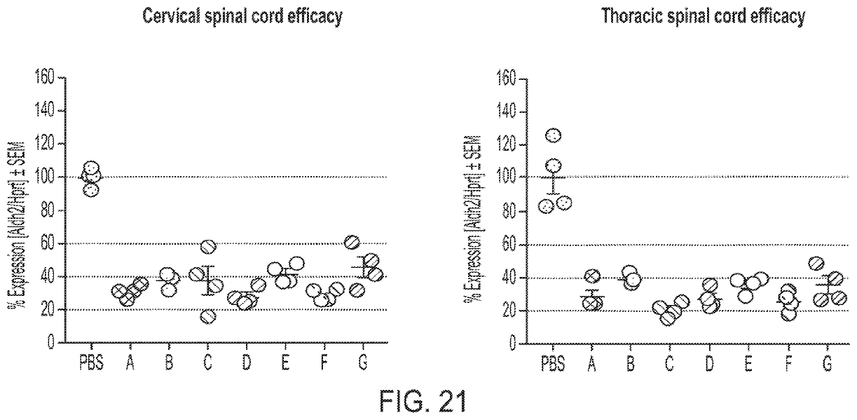

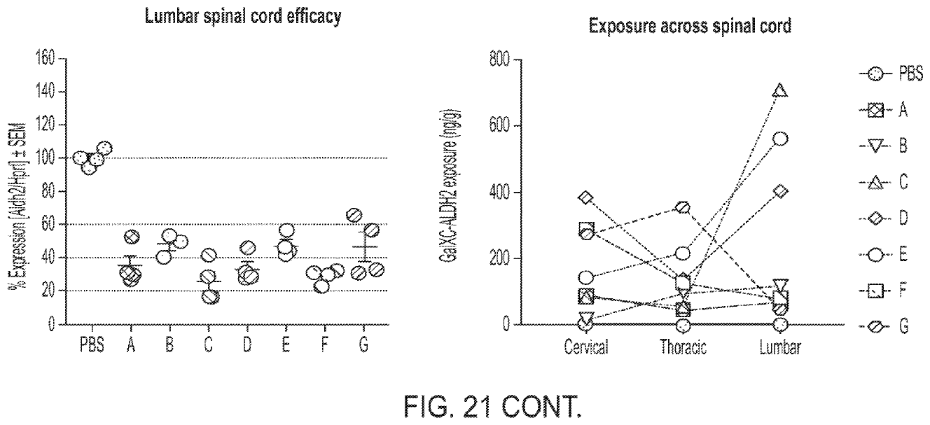

All conjugates were effective in reducing ALDH2 mRNA level in the frontal cortex (FIG. 14), striatum (FIG. 15), somatosensory cortex (FIG. 16), hippocampus (FIG. 17), hypothalamus (FIG. 18), cerebellum (FIG. 19), and across the spinal cord (FIG. 21). A summary of relative exposure of the ALDH2 RNAi oligonucleotide derivatives across different brain regions is shown in FIG. 20.

The results indicate that non-GalNAc-conjugated RNAi oligonucleotides are inactive in the liver after two weeks and GalNAc conjugation is not required for neural cell uptake and conjugate efficacy. All derivatives showed roughly comparable distribution across the brain and spinal cord (although there was up to a 10-fold difference in absolute accumulation levels between some groups). Proximal to the site of infusion (somatosensory cortex and hippocampus), enhanced activity (by 20-40%) were observed with non-GalNAc-conjugated constructs (Conjugates C-G). Distal from the site of infusion (frontal cortex, striatum, hypothalamus, cerebellum, spinal cord), comparable activity between GalNAc-conjugated and non-conjugated derivatives were observed.

In general, Conjugate E (2′-OH-substituted tetraloop) is less efficacious. The highest overall exposure was observed with Conjugate G (2′-adem-substituted tetraloop) and Conjugate F (2′-MOE-substituted tetraloop).

Target Sequences in the ALDH2 gene are provided in Table 5.

TABLE 5
Sequences of Hotspots

| Hotspot | | |
|---|---|---|
| Position | | |
| In Human | | SEQ |
| ALDH2 | | ID |
| mRNA | Sequence | NO. |
| 181-273 | AACCAGCAGCCCGAGGTCTTCTGCAAC | 601 |
| | CAGATTTTCATAAACAATGAATGGCAC | |
| | GATGCCGTCAGCAGGAAAACATTCCCC | |
| | ACCGTCAATCCG | |
| 445-539 | ACCTACCTGGCGGCCTTGGAGACCCTG | 602 |
| | GACAATGGCAAGCCCTATGTCATCTCC | |
| | TACCTGGTGGATTTGGACATGGTCCTC | |
| | AAATGTCTCCGGTATTATGC | |
| 646-696 | CCGTGGAATTTCCCGCTCCTGATGCAA | 603 |
| | GCATGGAAGCTGGGCCCAGCCTTG | |
| 691-749 | GCCTTGGCAACTGGAAACGTGGTTGTG | 604 |
| | ATGAAGGTAGCTGAGCAGACACCCCTC | |
| | ACCGC | |
| 1165-1235 | GAGCAGGGGCCGCAGGTGGATGAAACT | 605 |
| | CAGTTTAAGAAGATCCTCGGCTACATC | |
| | AACACGGGGAAGCAAGA | |
| 1770-1821 | TCTCTTGGGTCAAGAAAGTTCTAGAAT | 606 |
| | TTGAATTGATAAACATGGTGGGTTG | |
| 1824-1916 | TGAGGGTAAGAGTATATGAGGAACCTT | 607 |
| | TTAAACGACAACAATACTGCTAGCTTT | |
| | CAGGATGATTTTTAAAAAATAGATTCA | |
| | AATGTGTTATCC | |

### Description of Oligonucleotide Nomenclature

All oligonucleotides described herein are designated either SN₁-ASN₂-MN₃. The following designations apply:
N₁: sequence identifier number of the sense strand sequence
N₂: sequence identifier number of the antisense strand sequence

For example, S27-AS317 represents an oligonucleotide with a sense sequence that is set forth by SEQ ID NO: 27, an antisense sequence that is set forth by SEQ ID NO: 317.

### REFERENCES

1. Fire A. and Xu S, “Potent and specific genetic interference by double-stranded RNA in Caenorhabditis elegans,” Nature, 1998, 391(6669):806-811.
2. Hannon, G. J., “RNA interference,” Nature, 2002, 418:244-251.
3. Xia et al., “RNAi suppresses polyglutamine-induced neurodegeneration in a model of spinocerebellar ataxia,” Nat Med., 2004, 10(8):816-820.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize or be able to ascertain using no more than routine experimentation, many equivalents to the specific embodiments of the invention described herein. Such equivalents are intended to be encompassed by the following claims. The contents of all references, patents, and patent applications cited throughout this application are hereby incorporated by reference.

APPENDIX A

| | | S | | AS |
|---|---|---|---|---|
| | | SEQ | | SEQ |
| App | Sense Sequence/ | ID | Antisense | ID |
| Name | mRNA seq | NO | Sequence | NO |
| S1- | GAGGUCUUCUGCAACCAG | 1 | UGAAAAUCUGGUUGCAGA | 291 |
| AS291 | AUUUUCA | | AGACCUCGG | |
| S2- | AGGUCUUCUGCAACCAGA | 2 | AUGAAAAUCUGGUUGCAG | 292 |
| AS292 | UUUUCAT | | AAGACCUCG | |
| S3- | GUCUUCUGCAACCAGAUU | 3 | UUAUGAAAAUCUGGUUGC | 293 |
| AS293 | UUCAUAA | | AGAAGACCU | |
| S4- | CUUCUGCAACCAGAUUUU | 4 | GUUUAUGAAAAUCUGGUU | 294 |
| AS294 | CAUAAAC | | GCAGAAGAC | |
| S5- | UUCUGCAACCAGAUUUUC | 5 | UGUUUAUGAAAAUCUGGU | 295 |
| AS295 | AUAAACA | | UGCAGAAGA | |
| S6- | UCUGCAACCAGAUUUUCA | 6 | UUGUUUAUGAAAAUCUGG | 296 |
| AS296 | UAAACAA | | UUGCAGAAG | |
| S7- | CUGCAACCAGAUUUUCAU | 7 | AUUGUUUAUGAAAAUCUG | 297 |
| AS297 | AAACAAT | | GUUGCAGAA | |
| S8- | UGCAACCAGAUUUUCAUA | 8 | CAUUGUUUAUGAAAAUCU | 298 |
| AS298 | AACAATG | | GGUUGCAGA | |
| S9- | GCAACCAGAUUUUCAUAA | 9 | UCAUUGUUUAUGAAAAUC | 299 |
| AS299 | ACAAUGA | | UGGUUGCAG | |
| S10- | CAACCAGAUUUUCAUAAA | 10 | UUCAUUGUUUAUGAAAAU | 300 |
| AS300 | CAAUGAA | | CUGGUUGCA | |
| S11- | AACCAGAUUUUCAUAAAC | 11 | AUUCAUUGUUUAUGAAAA | 301 |
| AS301 | AAUGAAT | | UCUGGUUGC | |
| S12- | ACCAGAUUUUCAUAAACA | 12 | CAUUCAUUGUUUAUGAAA | 302 |
| AS302 | AUGAATG | | AUCUGGUUG | |
| S13- | CCAGAUUUUCAUAAACAA | 13 | CCAUUCAUUGUUUAUGAA | 303 |
| AS303 | UGAAUGG | | AAUCUGGUU | |
| S14- | CAGAUUUUCAUAAACAAU | 14 | GCCAUUCAUUGUUUAUGA | 304 |
| AS304 | GAAUGGC | | AAAUCUGGU | |
| S17- | AGAUUUUCAUAAACAAUG | 17 | UGCCAUUCAUUGUUUAUG | 307 |
| AS307 | AAUGGCA | | AAAAUCUGG | |
| S18- | GAUUUUCAUAAACAAUGA | 18 | GUGCCAUUCAUUGUUUAU | 308 |
| AS308 | AUGGCAC | | GAAAAUCUG | |
| S19- | GCCGUCAGCAGGAAAACA | 19 | UGGGGAAUGUUUUCCUGC | 309 |
| AS309 | UUCCCCA | | UGACGGCAU | |
| S20- | CCGUCAGCAGGAAAACAU | 20 | GUGGGGAAUGUUUUCCUG | 310 |
| AS310 | UCCCCAC | | CUGACGGCA | |
| S21- | GGCCUUGGAGACCCUGGA | 21 | GCCAUUGUCCAGGGUCUC | 311 |
| AS311 | CAAUGGC | | CAAGGCCGC | |
| S22- | GCCUUGGAGACCCUGGAC | 22 | UGCCAUUGUCCAGGGUCU | 312 |
| AS312 | AAUGGCA | | CCAAGGCCG | |
| S23- | CCUUGGAGACCCUGGACA | 23 | UUGCCAUUGUCCAGGGUC | 313 |
| AS313 | AUGGCAA | | UCCAAGGCC | |
| S24- | UACCUGGUGGAUUUGGAC | 24 | GGACCAUGUCCAAAUCCA | 314 |
| AS314 | AUGGUCC | | CCAGGUAGG | |
| S25- | ACCUGGUGGAUUUGGACA | 25 | AGGACCAUGUCCAAAUCC | 315 |
| AS315 | UGGUCCT | | ACCAGGUAG | |
| S26- | CCUGGUGGAUUUGGACAU | 26 | GAGGACCAUGUCCAAAUC | 316 |
| AS316 | GGUCCTC | | CACCAGGUA | |
| S27- | CUGGUGGAUUUGGACAUG | 27 | UGAGGACCAUGUCCAAAU | 317 |
| AS317 | GUCCUCA | | CCACCAGGU | |
| S28- | UGGUGGAUUUGGACAUG | 28 | UUGAGGACCAUGUCCAAA | 318 |
| AS318 | GUCCUCAA | | UCCACCAGG | |
| S29- | GGUGGAUUUGGACAUGG | 29 | UUUGAGGACCAUGUCCAA | 319 |
| AS319 | UCCUCAAA | | AUCCACCAG | |
| S30- | GUGGAUUUGGACAUGGUC | 30 | AUUUGAGGACCAUGUCCA | 320 |
| AS320 | CUCAAAT | | AAUCCACCA | |
| S31- | UGGAUUUGGACAUGGUCC | 31 | CAUUUGAGGACCAUGUCC | 321 |
| AS321 | UCAAATG | | AAAUCCACC | |
| S32- | GAUUUGGACAUGGUCCUC | 32 | GACAUUUGAGGACCAUGU | 322 |
| AS322 | AAAUGTC | | CCAAAUCCA | |
| S33- | UUCCCGCUCCUGAUGCAA | 33 | UCCAUGCUUGCAUCAGGA | 323 |
| AS323 | GCAUGGA | | GCGGGAAAU | |
| S34- | UCCCGCUCCUGAUGCAAG | 34 | UUCCAUGCUUGCAUCAGG | 324 |
| AS324 | CAUGGAA | | AGCGGGAAA | |
| S35- | CCCGCUCCUGAUGCAAGC | 35 | CUUCCAUGCUUGCAUCAG | 325 |
| AS325 | AUGGAAG | | GAGCGGGAA | |
| S36- | CCGCUCCUGAUGCAAGCA | 36 | GCUUCCAUGCUUGCAUCA | 326 |
| AS326 | UGGAAGC | | GGAGCGGGA | |
| S37- | CGCUCCUGAUGCAAGCAU | 37 | AGCUUCCAUGCUUGCAUC | 327 |
| AS327 | GGAAGCT | | AGGAGCGGG | |
| S38- | GCUCCUGAUGCAAGCAUG | 38 | CAGCUUCCAUGCUUGCAU | 328 |
| AS328 | GAAGCTG | | CAGGAGCGG | |
| S39- | CUCCUGAUGCAAGCAUGG | 39 | CCAGCUUCCAUGCUUGCA | 329 |
| AS329 | AAGCUGG | | UCAGGAGCG | |
| S40- | UCCUGAUGCAAGCAUGGA | 40 | CCCAGCUUCCAUGCUUGC | 330 |
| AS330 | AGCUGGG | | AUCAGGAGC | |
| S41- | AACUGGAAACGUGGUUGU | 41 | CUUCAUCACAACCACGUU | 331 |
| AS331 | GAUGAAG | | UCCAGUUGC | |
| S42- | ACUGGAAACGUGGUUGUG | 42 | CCUUCAUCACAACCACGU | 332 |
| AS332 | AUGAAGG | | UUCCAGUUG | |
| S43- | CUGGAAACGUGGUUGUGA | 43 | ACCUUCAUCACAACCACG | 333 |
| AS333 | UGAAGGT | | UUUCCAGUU | |
| S44- | UGGAAACGUGGUUGUGA | 44 | UACCUUCAUCACAACCAC | 334 |
| AS334 | UGAAGGTA | | GUUUCCAGU | |
| S45- | GGAAACGUGGUUGUGAU | 45 | CUACCUUCAUCACAACCA | 335 |
| AS335 | GAAGGUAG | | CGUUUCCAG | |
| S46- | GAAACGUGGUUGUGAUG | 46 | GCUACCUUCAUCACAACC | 336 |
| AS336 | AAGGUAGC | | ACGUUUCCA | |
| S47- | AACGUGGUUGUGAUGAA | 47 | CAGCUACCUUCAUCACAA | 337 |
| AS337 | GGUAGCTG | | CCACGUUUC | |
| S48- | ACGUGGUUGUGAUGAAG | 48 | UCAGCUACCUUCAUCACA | 338 |
| AS338 | GUAGCUGA | | ACCACGUUU | |
| S49- | CGUGGUUGUGAUGAAGG | 49 | CUCAGCUACCUUCAUCAC | 339 |
| AS339 | UAGCUGAG | | AACCACGUU | |
| S50- | GUUGUGAUGAAGGUAGC | 50 | UCUGCUCAGCUACCUUCA | 340 |
| AS340 | UGAGCAGA | | UCACAACCA | |
| S51- | GUGAUGAAGGUAGCUGA | 51 | GUGUCUGCUCAGCUACCU | 341 |
| AS341 | GCAGACAC | | UCAUCACAA | |
| S52- | AGGAUGUGGACAAAGUG | 52 | GUGAAUGCCACUUUGUCC | 342 |
| AS342 | GCAUUCAC | | ACAUCCUCA | |
| S53- | GGGAGCAGCAACCUCAAG | 53 | UCACUCUCUUGAGGUUGC | 343 |
| AS343 | AGAGUGA | | UGCUCCCAG | |
| S54- | GGAGCAGCAACCUCAAGA | 54 | GUCACUCUCUUGAGGUUG | 344 |
| AS344 | GAGUGAC | | CUGCUCCCA | |
| S55- | GAGCAGCAACCUCAAGAG | 55 | GGUCACUCUCUUGAGGUU | 345 |
| AS345 | AGUGACC | | GCUGCUCCC | |
| S56- | AGCAGCAACCUCAAGAGA | 56 | AGGUCACUCUCUUGAGGU | 346 |
| AS346 | GUGACCT | | UGCUGCUCC | |
| S57- | GCAGCAACCUCAAGAGAG | 57 | AAGGUCACUCUCUUGAGG | 347 |
| AS347 | UGACCTT | | UUGCUGCUC | |
| S58- | GCCCUGUUCUUCAACCAG | 58 | ACUGGCCCUGGUUGAAGA | 348 |
| AS348 | GGCCAGT | | ACAGGGCGA | |
| S59- | CCCUGUUCUUCAACCAGG | 59 | CACUGGCCCUGGUUGAAG | 349 |
| AS349 | GCCAGTG | | AACAGGGCG | |
| S60- | CCUGUUCUUCAACCAGGG | 60 | GCACUGGCCCUGGUUGAA | 350 |
| AS350 | CCAGUGC | | GAACAGGGC | |
| S61- | CUGUUCUUCAACCAGGGC | 61 | AGCACUGGCCCUGGUUGA | 351 |
| AS351 | CAGUGCT | | AGAACAGGG | |
| S62- | UGUUCUUCAACCAGGGCC | 62 | CAGCACUGGCCCUGGUUG | 352 |
| AS352 | AGUGCTG | | AAGAACAGG | |
| S63- | GUUCUUCAACCAGGGCCA | 63 | GCAGCACUGGCCCUGGUU | 353 |
| AS353 | GUGCUGC | | GAAGAACAG | |
| S64- | UUCUUCAACCAGGGCCAG | 64 | AGCAGCACUGGCCCUGGU | 354 |
| AS354 | UGCUGCT | | UGAAGAACA | |
| S65- | CUUCAACCAGGGCCAGUG | 65 | ACAGCAGCACUGGCCCUG | 355 |
| AS355 | CUGCUGT | | GUUGAAGAA | |
| S66- | UUCAACCAGGGCCAGUGC | 66 | CACAGCAGCACUGGCCCU | 356 |
| AS356 | UGCUGTG | | GGUUGAAGA | |
| S67- | CAACCAGGGCCAGUGCUG | 67 | GGCACAGCAGCACUGGCC | 357 |
| AS357 | CUGUGCC | | CUGGUUGAA | |
| S68- | GGCUCCCGGACCUUCGUG | 68 | CCUCCUGCACGAAGGUCC | 358 |
| AS358 | CAGGAGG | | GGGAGCCGG | |
| S69- | GCUCCCGGACCUUCGUGC | 69 | UCCUCCUGCACGAAGGUC | 359 |
| AS359 | AGGAGGA | | CGGGAGCCG | |
| S70- | CUCCCGGACCUUCGUGCA | 70 | GUCCUCCUGCACGAAGGU | 360 |
| AS360 | GGAGGAC | | CCGGGAGCC | |
| S71- | UCCCGGACCUUCGUGCAG | 71 | UGUCCUCCUGCACGAAGG | 361 |
| AS361 | GAGGACA | | UCCGGGAGC | |
| S72- | CCCGGACCUUCGUGCAGG | 72 | AUGUCCUCCUGCACGAAG | 362 |
| AS362 | AGGACAT | | GUCCGGGAG | |
| S73- | CCGGACCUUCGUGCAGGA | 73 | GAUGUCCUCCUGCACGAA | 363 |
| AS363 | GGACATC | | GGUCCGGGA | |
| S74- | GGAGGACAUCUAUGAUGA | 74 | CACAAACUCAUCAUAGAU | 364 |
| AS364 | GUUUGTG | | GUCCUCCUG | |
| S75- | CGGGCCAAGUCUCGGGUG | 75 | UCCCGACCACCCGAGACU | 365 |
| AS365 | GUCGGGA | | UGGCCCGGG | |
| S76- | GGGCCAAGUCUCGGGUGG | 76 | UUCCCGACCACCCGAGAC | 366 |
| AS366 | UCGGGAA | | UUGGCCCGG | |
| S77- | GCAGGUGGAUGAAACUCA | 77 | CUUAAACUGAGUUUCAUC | 367 |
| AS367 | GUUUAAG | | CACCUGCGG | |
| S78- | CAGGUGGAUGAAACUCAG | 78 | UCUUAAACUGAGUUUCAU | 368 |
| AS368 | UUUAAGA | | CCACCUGCG | |
| S79- | AGGUGGAUGAAACUCAGU | 79 | UUCUUAAACUGAGUUUCA | 369 |
| AS369 | UUAAGAA | | UCCACCUGC | |
| S80- | GGUGGAUGAAACUCAGUU | 80 | CUUCUUAAACUGAGUUUC | 370 |
| AS370 | UAAGAAG | | AUCCACCUG | |
| S81- | GUGGAUGAAACUCAGUUU | 81 | UCUUCUUAAACUGAGUUU | 371 |
| AS371 | AAGAAGA | | CAUCCACCU | |
| S82- | UGGAUGAAACUCAGUUUA | 82 | AUCUUCUUAAACUGAGUU | 372 |
| AS372 | AGAAGAT | | UCAUCCACC | |
| S83- | GGAUGAAACUCAGUUUAA | 83 | GAUCUUCUUAAACUGAGU | 373 |
| AS373 | GAAGATC | | UUCAUCCAC | |
| S84- | GAUGAAACUCAGUUUAAG | 84 | GGAUCUUCUUAAACUGAG | 374 |
| AS374 | AAGAUCC | | UUUCAUCCA | |
| S85- | AUGAAACUCAGUUUAAGA | 85 | AGGAUCUUCUUAAACUGA | 375 |
| AS375 | AGAUCCT | | GUUUCAUCC | |
| S86- | UGAAACUCAGUUUAAGAA | 86 | GAGGAUCUUCUUAAACUG | 376 |
| AS376 | GAUCCTC | | AGUUUCAUC | |
| S87- | GAAACUCAGUUUAAGAAG | 87 | CGAGGAUCUUCUUAAACU | 377 |
| AS377 | AUCCUCG | | GAGUUUCAU | |
| S88- | AAACUCAGUUUAAGAAGA | 88 | CCGAGGAUCUUCUUAAAC | 378 |
| AS378 | UCCUCGG | | UGAGUUUCA | |
| S89- | AACUCAGUUUAAGAAGAU | 89 | GCCGAGGAUCUUCUUAAA | 379 |
| AS379 | CCUCGGC | | CUGAGUUUC | |
| S90- | ACUCAGUUUAAGAAGAUC | 90 | AGCCGAGGAUCUUCUUAA | 380 |
| AS380 | CUCGGCT | | ACUGAGUUU | |
| S91- | CUCAGUUUAAGAAGAUCC | 91 | UAGCCGAGGAUCUUCUUA | 381 |
| AS381 | UCGGCTA | | AACUGAGUU | |
| S92- | UCAGUUUAAGAAGAUCCU | 92 | GUAGCCGAGGAUCUUCUU | 382 |
| AS382 | CGGCUAC | | AAACUGAGU | |
| S93- | CAGUUUAAGAAGAUCCUC | 93 | UGUAGCCGAGGAUCUUCU | 383 |
| AS383 | GGCUACA | | UAAACUGAG | |
| S94- | AGUUUAAGAAGAUCCUCG | 94 | AUGUAGCCGAGGAUCUUC | 384 |
| AS384 | GCUACAT | | UUAAACUGA | |
| S95- | GUUUAAGAAGAUCCUCGG | 95 | GAUGUAGCCGAGGAUCUU | 385 |
| AS385 | CUACATC | | CUUAAACUG | |
| S96- | UUUAAGAAGAUCCUCGGC | 96 | UGAUGUAGCCGAGGAUCU | 386 |
| AS386 | UACAUCA | | UCUUAAACU | |
| S97- | UUAAGAAGAUCCUCGGCU | 97 | UUGAUGUAGCCGAGGAUC | 387 |
| AS387 | ACAUCAA | | UUCUUAAAC | |
| S98- | UAAGAAGAUCCUCGGCUA | 98 | GUUGAUGUAGCCGAGGAU | 388 |
| AS388 | CAUCAAC | | CUUCUUAAA | |
| S99- | AAGAAGAUCCUCGGCUAC | 99 | UGUUGAUGUAGCCGAGGA | 389 |
| AS389 | AUCAACA | | UCUUCUUAA | |
| S100- | AGAAGAUCCUCGGCUACA | 100 | GUGUUGAUGUAGCCGAGG | 390 |
| AS390 | UCAACAC | | AUCUUCUUA | |
| S101- | GAAGAUCCUCGGCUACAU | 101 | CGUGUUGAUGUAGCCGAG | 391 |
| AS391 | CAACACG | | GAUCUUCUU | |
| S102- | AAGAUCCUCGGCUACAUC | 102 | CCGUGUUGAUGUAGCCGA | 392 |
| AS392 | AACACGG | | GGAUCUUCU | |
| S103- | AGAUCCUCGGCUACAUCA | 103 | CCCGUGUUGAUGUAGCCG | 393 |
| AS393 | ACACGGG | | AGGAUCUUC | |
| S104- | UGCUGCUGACCGUGGUUA | 104 | GAUGAAGUAACCACGGUC | 394 |
| AS394 | CUUCATC | | AGCAGCAAU | |
| S105- | GCUGCUGACCGUGGUUAC | 105 | GGAUGAAGUAACCACGGU | 395 |
| AS395 | UUCAUCC | | CAGCAGCAA | |
| S106- | CUGCUGACCGUGGUUACU | 106 | UGGAUGAAGUAACCACGG | 396 |
| AS396 | UCAUCCA | | UCAGCAGCA | |
| S107- | GCUGACCGUGGUUACUUC | 107 | GCUGGAUGAAGUAACCAC | 397 |
| AS397 | AUCCAGC | | GGUCAGCAG | |
| S108- | CCAGUGAUGCAGAUCCUG | 108 | UGAACUUCAGGAUCUGCA | 398 |
| AS398 | AAGUUCA | | UCACUGGCC | |
| S109- | AGUGAUGCAGAUCCUGAA | 109 | CUUGAACUUCAGGAUCUG | 399 |
| AS399 | GUUCAAG | | CAUCACUGG | |
| S110- | GUGAUGCAGAUCCUGAAG | 110 | UCUUGAACUUCAGGAUCU | 400 |
| AS400 | UUCAAGA | | GCAUCACUG | |
| S111- | UGAUGCAGAUCCUGAAGU | 111 | GUCUUGAACUUCAGGAUC | 401 |
| AS401 | UCAAGAC | | UGCAUCACU | |
| S112- | GAUGCAGAUCCUGAAGUU | 112 | GGUCUUGAACUUCAGGAU | 402 |
| AS402 | CAAGACC | | CUGCAUCAC | |
| S113- | AUGCAGAUCCUGAAGUUC | 113 | UGGUCUUGAACUUCAGGA | 403 |
| AS403 | AAGACCA | | UCUGCAUCA | |
| S114- | GCAGAUCCUGAAGUUCAA | 114 | UAUGGUCUUGAACUUCAG | 404 |
| AS404 | GACCATA | | GAUCUGCAU | |
| S115- | CAGAUCCUGAAGUUCAAG | 115 | CUAUGGUCUUGAACUUCA | 405 |
| AS405 | ACCAUAG | | GGAUCUGCA | |
| S116- | AGAUCCUGAAGUUCAAGA | 116 | UCUAUGGUCUUGAACUUC | 406 |
| AS406 | CCAUAGA | | AGGAUCUGC | |
| S117- | GAUCCUGAAGUUCAAGAC | 117 | CUCUAUGGUCUUGAACUU | 407 |
| AS407 | CAUAGAG | | CAGGAUCUG | |
| S118- | UCCUGAAGUUCAAGACCA | 118 | UCCUCUAUGGUCUUGAAC | 408 |
| AS408 | UAGAGGA | | UUCAGGAUC | |
| S119- | AAGUUCAAGACCAUAGAG | 119 | CAACCUCCUCUAUGGUCU | 409 |
| AS409 | GAGGUTG | | UGAACUUCA | |
| S120- | GCUGUCUUCACAAAGGAU | 120 | UGUCCAAAUCCUUUGUGA | 410 |
| AS410 | UUGGACA | | AGACAGCUG | |
| S121- | GUCUUCACAAAGGAUUUG | 121 | CCUUGUCCAAAUCCUUUG | 411 |
| AS411 | GACAAGG | | UGAAGACAG | |
| S122- | GCAGGCAUACACUGAAGU | 122 | AGUUUUCACUUCAGUGUA | 412 |
| AS412 | GAAAACT | | UGCCUGCAG | |
| S123- | CAGGCAUACACUGAAGUG | 123 | CAGUUUUCACUUCAGUGU | 413 |
| AS413 | AAAACTG | | AUGCCUGCA | |
| S124- | AGGCAUACACUGAAGUGA | 124 | ACAGUUUUCACUUCAGUG | 414 |
| AS414 | AAACUGT | | UAUGCCUGC | |
| S125- | GGCAUACACUGAAGUGAA | 125 | GACAGUUUUCACUUCAGU | 415 |
| AS415 | AACUGTC | | GUAUGCCUG | |
| S126- | GCAUACACUGAAGUGAAA | 126 | UGACAGUUUUCACUUCAG | 416 |
| AS416 | ACUGUCA | | UGUAUGCCU | |
| S127- | AUACACUGAAGUGAAAAC | 127 | UGUGACAGUUUUCACUUC | 417 |
| AS417 | UGUCACA | | AGUGUAUGC | |
| S128- | UACACUGAAGUGAAAACU | 128 | CUGUGACAGUUUUCACUU | 418 |
| AS418 | GUCACAG | | CAGUGUAUG | |
| S129- | CUGAAGUGAAAACUGUCA | 129 | UUGACUGUGACAGUUUUC | 419 |
| AS419 | CAGUCAA | | ACUUCAGUG | |
| S130- | GUCAAAGUGCCUCAGAAG | 130 | AUGAGUUCUUCUGAGGCA | 420 |
| AS420 | AACUCAT | | CUUUGACUG | |
| S131- | CAAAGUGCCUCAGAAGAA | 131 | UUAUGAGUUCUUCUGAGG | 421 |
| AS421 | CUCAUAA | | CACUUUGAC | |
| S132- | AAGUGCCUCAGAAGAACU | 132 | UCUUAUGAGUUCUUCUGA | 422 |
| AS422 | CAUAAGA | | GGCACUUUG | |
| S133- | AGUGCCUCAGAAGAACUC | 133 | UUCUUAUGAGUUCUUCUG | 423 |
| AS423 | AUAAGAA | | AGGCACUUU | |
| S134- | GUGCCUCAGAAGAACUCA | 134 | AUUCUUAUGAGUUCUUCU | 424 |
| AS424 | UAAGAAT | | GAGGCACUU | |
| S135- | UGCCUCAGAAGAACUCAU | 135 | GAUUCUUAUGAGUUCUUC | 425 |
| AS425 | AAGAATC | | UGAGGCACU | |
| S136- | CCUCAGAAGAACUCAUAA | 136 | AUGAUUCUUAUGAGUUCU | 426 |
| AS426 | GAAUCAT | | UCUGAGGCA | |
| S137- | CUCAGAAGAACUCAUAAG | 137 | CAUGAUUCUUAUGAGUUC | 427 |
| AS427 | AAUCATG | | UUCUGAGGC | |
| S138- | UCAGAAGAACUCAUAAGA | 138 | GCAUGAUUCUUAUGAGUU | 428 |
| AS428 | AUCAUGC | | CUUCUGAGG | |
| S139- | CAGAAGAACUCAUAAGAA | 139 | UGCAUGAUUCUUAUGAGU | 429 |
| AS429 | UCAUGCA | | UCUUCUGAG | |
| S140- | AGAAGAACUCAUAAGAAU | 140 | UUGCAUGAUUCUUAUGAG | 430 |
| AS430 | CAUGCAA | | UUCUUCUGA | |
| S141- | GAAGAACUCAUAAGAAUC | 141 | CUUGCAUGAUUCUUAUGA | 431 |
| AS431 | AUGCAAG | | GUUCUUCUG | |
| S142- | AAGAACUCAUAAGAAUCA | 142 | GCUUGCAUGAUUCUUAUG | 432 |
| AS432 | UGCAAGC | | AGUUCUUCU | |
| S143- | GAACUCAUAAGAAUCAUG | 143 | AAGCUUGCAUGAUUCUUA | 433 |
| AS433 | CAAGCTT | | UGAGUUCUU | |
| S144- | AACUCAUAAGAAUCAUGC | 144 | GAAGCUUGCAUGAUUCUU | 434 |
| AS434 | AAGCUTC | | AUGAGUUCU | |
| S145- | CCCUCAGCCAUUGAUGGA | 145 | UGAACUUUCCAUCAAUGG | 435 |
| AS435 | AAGUUCA | | CUGAGGGAG | |
| S146- | CCUCAGCCAUUGAUGGAA | 146 | CUGAACUUUCCAUCAAUG | 436 |
| AS436 | AGUUCAG | | GCUGAGGGA | |
| S147- | UCAGCCAUUGAUGGAAAG | 147 | UGCUGAACUUUCCAUCAA | 437 |
| AS437 | UUCAGCA | | UGGCUGAGG | |
| S148- | CAGCCAUUGAUGGAAAGU | 148 | UUGCUGAACUUUCCAUCA | 438 |
| AS438 | UCAGCAA | | AUGGCUGAG | |
| S149- | AGCCAUUGAUGGAAAGUU | 149 | CUUGCUGAACUUUCCAUC | 439 |
| AS439 | CAGCAAG | | AAUGGCUGA | |
| S150- | GCCAUUGAUGGAAAGUUC | 150 | UCUUGCUGAACUUUCCAU | 440 |
| AS440 | AGCAAGA | | CAAUGGCUG | |
| S151- | CCAUUGAUGGAAAGUUCA | 151 | AUCUUGCUGAACUUUCCA | 441 |
| AS441 | GCAAGAT | | UCAAUGGCU | |
| S152- | CAUUGAUGGAAAGUUCAG | 152 | GAUCUUGCUGAACUUUCC | 442 |
| AS442 | CAAGATC | | AUCAAUGGC | |
| S153- | AUUGAUGGAAAGUUCAGC | 153 | UGAUCUUGCUGAACUUUC | 443 |
| AS443 | AAGAUCA | | CAUCAAUGG | |
| S154- | UUGAUGGAAAGUUCAGCA | 154 | CUGAUCUUGCUGAACUUU | 444 |
| AS444 | AGAUCAG | | CCAUCAAUG | |
| S155- | UGAUGGAAAGUUCAGCAA | 155 | GCUGAUCUUGCUGAACUU | 445 |
| AS445 | GAUCAGC | | UCCAUCAAU | |
| S156- | GAUGGAAAGUUCAGCAAG | 156 | UGCUGAUCUUGCUGAACU | 446 |
| AS446 | AUCAGCA | | UUCCAUCAA | |
| S157- | AUGGAAAGUUCAGCAAGA | 157 | UUGCUGAUCUUGCUGAAC | 447 |
| AS447 | UCAGCAA | | UUUCCAUCA | |
| S158- | UGGAAAGUUCAGCAAGAU | 158 | GUUGCUGAUCUUGCUGAA | 448 |
| AS448 | CAGCAAC | | CUUUCCAUC | |
| S159- | GGAAAGUUCAGCAAGAUC | 159 | UGUUGCUGAUCUUGCUGA | 449 |
| AS449 | AGCAACA | | ACUUUCCAU | |
| S160- | GAAAGUUCAGCAAGAUCA | 160 | UUGUUGCUGAUCUUGCUG | 450 |
| AS450 | GCAACAA | | AACUUUCCA | |
| S161- | AAAGUUCAGCAAGAUCAG | 161 | UUUGUUGCUGAUCUUGCU | 451 |
| AS451 | CAACAAA | | GAACUUUCC | |
| S162- | AAGUUCAGCAAGAUCAGC | 162 | UUUUGUUGCUGAUCUUGC | 452 |
| AS452 | AACAAAA | | UGAACUUUC | |
| S163- | AUCAGCAACAAAACCAAG | 163 | CAUUUUUCUUGGUUUUGU | 453 |
| AS453 | AAAAATG | | UGCUGAUCU | |
| S164- | CAGCAACAAAACCAAGAA | 164 | AUCAUUUUUCUUGGUUUU | 454 |
| AS454 | AAAUGAT | | GUUGCUGAU | |
| S165- | AGCAACAAAACCAAGAAA | 165 | GAUCAUUUUUCUUGGUUU | 455 |
| AS455 | AAUGATC | | UGUUGCUGA | |
| S166- | ACAAAACCAAGAAAAAUG | 166 | CAAGGAUCAUUUUUCUUG | 456 |
| AS456 | AUCCUTG | | GUUUUGUUG | |
| S167- | CAAAACCAAGAAAAAUGA | 167 | GCAAGGAUCAUUUUUCUU | 457 |
| AS457 | UCCUUGC | | GGUUUUGUU | |
| S168- | AGAAAAAUGAUCCUUGCG | 168 | UUCAGCACGCAAGGAUCA | 458 |
| AS458 | UGCUGAA | | UUUUUCUUG | |
| S169- | AAAAAUGAUCCUUGCGUG | 169 | UAUUCAGCACGCAAGGAU | 459 |
| AS459 | CUGAATA | | CAUUUUUCU | |
| S170- | AAAAUGAUCCUUGCGUGC | 170 | AUAUUCAGCACGCAAGGA | 460 |
| AS460 | UGAAUAT | | UCAUUUUUC | |
| S171- | AAAUGAUCCUUGCGUGCU | 171 | GAUAUUCAGCACGCAAGG | 461 |
| AS461 | GAAUATC | | AUCAUUUUU | |
| S172- | AAUGAUCCUUGCGUGCUG | 172 | AGAUAUUCAGCACGCAAG | 462 |
| AS462 | AAUAUCT | | GAUCAUUUU | |
| S173- | AUGAUCCUUGCGUGCUGA | 173 | CAGAUAUUCAGCACGCAA | 463 |
| AS463 | AUAUCTG | | GGAUCAUUU | |
| S174- | UGAUCCUUGCGUGCUGAA | 174 | UCAGAUAUUCAGCACGCA | 464 |
| AS464 | UAUCUGA | | AGGAUCAUU | |
| S175- | GAUCCUUGCGUGCUGAAU | 175 | UUCAGAUAUUCAGCACGC | 465 |
| AS465 | AUCUGAA | | AAGGAUCAU | |
| S176- | UCCUUGCGUGCUGAAUAU | 176 | UUUUCAGAUAUUCAGCAC | 466 |
| AS466 | CUGAAAA | | GCAAGGAUC | |
| S177- | CCUUGCGUGCUGAAUAUC | 177 | CUUUUCAGAUAUUCAGCA | 467 |
| AS467 | UGAAAAG | | CGCAAGGAU | |
| S178- | CUUGCGUGCUGAAUAUCU | 178 | UCUUUUCAGAUAUUCAGC | 468 |
| AS468 | GAAAAGA | | ACGCAAGGA | |
| S179- | UUGCGUGCUGAAUAUCUG | 179 | CUCUUUUCAGAUAUUCAG | 469 |
| AS469 | AAAAGAG | | CACGCAAGG | |
| S180- | UGCGUGCUGAAUAUCUGA | 180 | UCUCUUUUCAGAUAUUCA | 470 |
| AS470 | AAAGAGA | | GCACGCAAG | |
| S181- | GCGUGCUGAAUAUCUGAA | 181 | UUCUCUUUUCAGAUAUUC | 471 |
| AS471 | AAGAGAA | | AGCACGCAA | |
| S182- | CGUGCUGAAUAUCUGAAA | 182 | UUUCUCUUUUCAGAUAUU | 472 |
| AS472 | AGAGAAA | | CAGCACGCA | |
| S183- | GUGCUGAAUAUCUGAAAA | 183 | AUUUCUCUUUUCAGAUAU | 473 |
| AS473 | GAGAAAT | | UCAGCACGC | |
| S184- | UGCUGAAUAUCUGAAAAG | 184 | AAUUUCUCUUUUCAGAUA | 474 |
| AS474 | AGAAATT | | UUCAGCACG | |
| S185- | GCUGAAUAUCUGAAAAGA | 185 | AAAUUUCUCUUUUCAGAU | 475 |
| AS475 | GAAAUTT | | AUUCAGCAC | |
| S186- | CUGAAUAUCUGAAAAGAG | 186 | AAAAUUUCUCUUUUCAGA | 476 |
| AS476 | AAAUUTT | | UAUUCAGCA | |
| S187- | UGAAUAUCUGAAAAGAG | 187 | AAAAAUUUCUCUUUUCAG | 477 |
| AS477 | AAAUUUTT | | AUAUUCAGC | |
| S188- | GAAUAUCUGAAAAGAGA | 188 | GAAAAAUUUCUCUUUUCA | 478 |
| AS478 | AAUUUUTC | | GAUAUUCAG | |
| S189- | AAUAUCUGAAAAGAGAA | 189 | GGAAAAAUUUCUCUUUUC | 479 |
| AS479 | AUUUUUCC | | AGAUAUUCA | |
| S190- | AUAUCUGAAAAGAGAAA | 190 | AGGAAAAAUUUCUCUUUU | 480 |
| AS480 | UUUUUCCT | | CAGAUAUUC | |
| S191- | AUCUGAAAAGAGAAAUU | 191 | GUAGGAAAAAUUUCUCUU | 481 |
| AS481 | UUUCCUAC | | UUCAGAUAU | |
| S192- | GAAAAGAGAAAUUUUUCC | 192 | UUUUGUAGGAAAAAUUUC | 482 |
| AS482 | UACAAAA | | UCUUUUCAG | |
| S193- | AAAAGAGAAAUUUUUCCU | 193 | AUUUUGUAGGAAAAAUUU | 483 |
| AS483 | ACAAAAT | | CUCUUUUCA | |
| S194- | AGAGAAAUUUUUCCUACA | 194 | GAGAUUUUGUAGGAAAAA | 484 |
| AS484 | AAAUCTC | | UUUCUCUUU | |
| S195- | GAGAAAUUUUUCCUACAA | 195 | AGAGAUUUUGUAGGAAAA | 485 |
| AS485 | AAUCUCT | | AUUUCUCUU | |
| S196- | AGAAAUUUUUCCUACAAA | 196 | AAGAGAUUUUGUAGGAAA | 486 |
| AS486 | AUCUCTT | | AAUUUCUCU | |
| S197- | CUUGGGUCAAGAAAGUUC | 197 | AAUUCUAGAACUUUCUUG | 487 |
| AS487 | UAGAATT | | ACCCAAGAG | |
| S198- | GGGUCAAGAAAGUUCUAG | 198 | UCAAAUUCUAGAACUUUC | 488 |
| AS488 | AAUUUGA | | UUGACCCAA | |
| S199- | GGUCAAGAAAGUUCUAGA | 199 | UUCAAAUUCUAGAACUUU | 489 |
| AS489 | AUUUGAA | | CUUGACCCA | |
| S200- | GUCAAGAAAGUUCUAGAA | 200 | AUUCAAAUUCUAGAACUU | 490 |
| AS490 | UUUGAAT | | UCUUGACCC | |
| S201- | UCAAGAAAGUUCUAGAAU | 201 | AAUUCAAAUUCUAGAACU | 491 |
| AS491 | UUGAATT | | UUCUUGACC | |
| S202- | CAAGAAAGUUCUAGAAUU | 202 | CAAUUCAAAUUCUAGAAC | 492 |
| AS492 | UGAAUTG | | UUUCUUGAC | |
| S203- | AAGAAAGUUCUAGAAUU | 203 | UCAAUUCAAAUUCUAGAA | 493 |
| AS493 | UGAAUUGA | | CUUUCUUGA | |
| S204- | AGAAAGUUCUAGAAUUU | 204 | AUCAAUUCAAAUUCUAGA | 494 |
| AS494 | GAAUUGAT | | ACUUUCUUG | |
| S205- | GAAAGUUCUAGAAUUUG | 205 | UAUCAAUUCAAAUUCUAG | 495 |
| AS495 | AAUUGATA | | AACUUUCUU | |
| S206- | AAAGUUCUAGAAUUUGA | 206 | UUAUCAAUUCAAAUUCUA | 496 |
| AS496 | AUUGAUAA | | GAACUUUCU | |
| S207- | AAGUUCUAGAAUUUGAA | 207 | UUUAUCAAUUCAAAUUCU | 497 |
| AS497 | UUGAUAAA | | AGAACUUUC | |
| S208- | AGUUCUAGAAUUUGAAU | 208 | GUUUAUCAAUUCAAAUUC | 498 |
| AS498 | UGAUAAAC | | UAGAACUUU | |
| S209- | GUUCUAGAAUUUGAAUU | 209 | UGUUUAUCAAUUCAAAUU | 499 |
| AS499 | GAUAAACA | | CUAGAACUU | |
| S210- | UUCUAGAAUUUGAAUUG | 210 | AUGUUUAUCAAUUCAAAU | 500 |
| AS500 | AUAAACAT | | UCUAGAACU | |
| S211- | UCUAGAAUUUGAAUUGA | 211 | CAUGUUUAUCAAUUCAAA | 501 |
| AS501 | UAAACATG | | UUCUAGAAC | |
| S212- | CUAGAAUUUGAAUUGAU | 212 | CCAUGUUUAUCAAUUCAA | 502 |
| AS502 | AAACAUGG | | AUUCUAGAA | |
| S213- | UAGAAUUUGAAUUGAUA | 213 | ACCAUGUUUAUCAAUUCA | 503 |
| AS503 | AACAUGGT | | AAUUCUAGA | |
| S214- | AGAAUUUGAAUUGAUAA | 214 | CACCAUGUUUAUCAAUUC | 504 |
| AS504 | ACAUGGTG | | AAAUUCUAG | |
| S215- | GAAUUUGAAUUGAUAAA | 215 | CCACCAUGUUUAUCAAUU | 505 |
| AS505 | CAUGGUGG | | CAAAUUCUA | |
| S216- | UAAGAGUAUAUGAGGAA | 216 | UUAAAAGGUUCCUCAUAU | 506 |
| AS506 | CCUUUUAA | | ACUCUUACC | |
| S217- | AAGAGUAUAUGAGGAACC | 217 | UUUAAAAGGUUCCUCAUA | 507 |
| AS507 | UUUUAAA | | UACUCUUAC | |
| S218- | AGAGUAUAUGAGGAACCU | 218 | GUUUAAAAGGUUCCUCAU | 508 |
| AS508 | UUUAAAC | | AUACUCUUA | |
| S219- | GAGUAUAUGAGGAACCUU | 219 | CGUUUAAAAGGUUCCUCA | 509 |
| AS509 | UUAAACG | | UAUACUCUU | |
| S220- | AGUAUAUGAGGAACCUUU | 220 | UCGUUUAAAAGGUUCCUC | 510 |
| AS510 | UAAACGA | | AUAUACUCU | |
| S221- | GUAUAUGAGGAACCUUUU | 221 | GUCGUUUAAAAGGUUCCU | 511 |
| AS511 | AAACGAC | | CAUAUACUC | |
| S222- | UAUAUGAGGAACCUUUUA | 222 | UGUCGUUUAAAAGGUUCC | 512 |
| AS512 | AACGACA | | UCAUAUACU | |
| S223- | AUGAGGAACCUUUUAAAC | 223 | UGUUGUCGUUUAAAAGGU | 513 |
| AS513 | GACAACA | | UCCUCAUAU | |
| S224- | GAGGAACCUUUUAAACGA | 224 | AUUGUUGUCGUUUAAAAG | 514 |
| AS514 | CAACAAT | | GUUCCUCAU | |
| S225- | AGGAACCUUUUAAACGAC | 225 | UAUUGUUGUCGUUUAAAA | 515 |
| AS515 | AACAATA | | GGUUCCUCA | |
| S226- | GAACCUUUUAAACGACAA | 226 | AGUAUUGUUGUCGUUUAA | 516 |
| AS516 | CAAUACT | | AAGGUUCCU | |
| S227- | AACCUUUUAAACGACAAC | 227 | CAGUAUUGUUGUCGUUUA | 517 |
| AS517 | AAUACTG | | AAAGGUUCC | |
| S228- | ACCUUUUAAACGACAACA | 228 | GCAGUAUUGUUGUCGUUU | 518 |
| AS518 | AUACUGC | | AAAAGGUUC | |
| S229- | CCUUUUAAACGACAACAA | 229 | AGCAGUAUUGUUGUCGUU | 519 |
| AS519 | UACUGCT | | UAAAAGGUU | |
| S230- | CUUUUAAACGACAACAAU | 230 | UAGCAGUAUUGUUGUCGU | 520 |
| AS520 | ACUGCTA | | UUAAAAGGU | |
| S231- | UAAACGACAACAAUACUG | 231 | AAGCUAGCAGUAUUGUUG | 521 |
| AS521 | CUAGCTT | | UCGUUUAAA | |
| S232- | AAACGACAACAAUACUGC | 232 | AAAGCUAGCAGUAUUGUU | 522 |
| AS522 | UAGCUTT | | GUCGUUUAA | |
| S233- | AACGACAACAAUACUGCU | 233 | GAAAGCUAGCAGUAUUGU | 523 |
| AS523 | AGCUUTC | | UGUCGUUUA | |
| S234- | CGACAACAAUACUGCUAG | 234 | CUGAAAGCUAGCAGUAUU | 524 |
| AS524 | CUUUCAG | | GUUGUCGUU | |
| S235- | GACAACAAUACUGCUAGC | 235 | CCUGAAAGCUAGCAGUAU | 525 |
| AS525 | UUUCAGG | | UGUUGUCGU | |
| S236- | ACAACAAUACUGCUAGCU | 236 | UCCUGAAAGCUAGCAGUA | 526 |
| AS526 | UUCAGGA | | UUGUUGUCG | |
| S237- | CAACAAUACUGCUAGCUU | 237 | AUCCUGAAAGCUAGCAGU | 527 |
| AS527 | UCAGGAT | | AUUGUUGUC | |
| S238- | AACAAUACUGCUAGCUUU | 238 | CAUCCUGAAAGCUAGCAG | 528 |
| AS528 | CAGGATG | | UAUUGUUGU | |
| S239- | ACAAUACUGCUAGCUUUC | 239 | UCAUCCUGAAAGCUAGCA | 529 |
| AS529 | AGGAUGA | | GUAUUGUUG | |
| S240- | CAAUACUGCUAGCUUUCA | 240 | AUCAUCCUGAAAGCUAGC | 530 |
| AS530 | GGAUGAT | | AGUAUUGUU | |
| S241- | AAUACUGCUAGCUUUCAG | 241 | AAUCAUCCUGAAAGCUAG | 531 |
| AS531 | GAUGATT | | CAGUAUUGU | |
| S242- | AUACUGCUAGCUUUCAGG | 242 | AAAUCAUCCUGAAAGCUA | 532 |
| AS532 | AUGAUTT | | GCAGUAUUG | |
| S243- | UACUGCUAGCUUUCAGGA | 243 | AAAAUCAUCCUGAAAGCU | 533 |
| AS533 | UGAUUTT | | AGCAGUAUU | |
| S244- | ACUGCUAGCUUUCAGGAU | 244 | AAAAAUCAUCCUGAAAGC | 534 |
| AS534 | GAUUUTT | | UAGCAGUAU | |
| S245- | CUGCUAGCUUUCAGGAUG | 245 | UAAAAAUCAUCCUGAAAG | 535 |
| AS535 | AUUUUTA | | CUAGCAGUA | |
| S246- | UGCUAGCUUUCAGGAUGA | 246 | UUAAAAAUCAUCCUGAAA | 536 |
| AS536 | UUUUUAA | | GCUAGCAGU | |
| S247- | GCUAGCUUUCAGGAUGAU | 247 | UUUAAAAAUCAUCCUGAA | 537 |
| AS537 | UUUUAAA | | AGCUAGCAG | |
| S248- | CUAGCUUUCAGGAUGAUU | 248 | UUUUAAAAAUCAUCCUGA | 538 |
| AS538 | UUUAAAA | | AAGCUAGCA | |
| S249- | AGCUUUCAGGAUGAUUUU | 249 | UUUUUUAAAAAUCAUCCU | 539 |
| AS539 | UAAAAAA | | GAAAGCUAG | |
| S250- | GCUUUCAGGAUGAUUUUU | 250 | AUUUUUUAAAAAUCAUCC | 540 |
| AS540 | AAAAAAT | | UGAAAGCUA | |
| S251- | CUUUCAGGAUGAUUUUUA | 251 | UAUUUUUUAAAAAUCAUC | 541 |
| AS541 | AAAAATA | | CUGAAAGCU | |
| S252- | UUUCAGGAUGAUUUUUA | 252 | CUAUUUUUUAAAAAUCAU | 542 |
| AS542 | AAAAAUAG | | CCUGAAAGC | |
| S253- | UUCAGGAUGAUUUUUAA | 253 | UCUAUUUUUUAAAAAUCA | 543 |
| AS543 | AAAAUAGA | | UCCUGAAAG | |
| S254- | UCAGGAUGAUUUUUAAA | 254 | AUCUAUUUUUUAAAAAUC | 544 |
| AS544 | AAAUAGAT | | AUCCUGAAA | |
| S255- | CAGGAUGAUUUUUAAAA | 255 | AAUCUAUUUUUUAAAAAU | 545 |
| AS545 | AAUAGATT | | CAUCCUGAA | |
| S256- | AGGAUGAUUUUUAAAAA | 256 | GAAUCUAUUUUUUAAAAA | 546 |
| AS546 | AUAGAUTC | | UCAUCCUGA | |
| S257- | GGAUGAUUUUUAAAAAA | 257 | UGAAUCUAUUUUUUAAAA | 547 |
| AS547 | UAGAUUCA | | AUCAUCCUG | |
| S258- | GAUGAUUUUUAAAAAAU | 258 | UUGAAUCUAUUUUUUAAA | 548 |
| AS548 | AGAUUCAA | | AAUCAUCCU | |
| S259- | AUGAUUUUUAAAAAAUA | 259 | UUUGAAUCUAUUUUUUAA | 549 |
| AS549 | GAUUCAAA | | AAAUCAUCC | |
| S260- | UGAUUUUUAAAAAAUAG | 260 | AUUUGAAUCUAUUUUUUA | 550 |
| AS550 | AUUCAAAT | | AAAAUCAUC | |
| S261- | GAUUUUUAAAAAAUAGA | 261 | CAUUUGAAUCUAUUUUUU | 551 |
| AS551 | UUCAAATG | | AAAAAUCAU | |
| S262- | AUUUUUAAAAAAUAGAU | 262 | ACAUUUGAAUCUAUUUUU | 552 |
| AS552 | UCAAAUGT | | UAAAAAUCA | |
| S263- | UUUUUAAAAAAUAGAUU | 263 | CACAUUUGAAUCUAUUUU | 553 |
| AS553 | CAAAUGTG | | UUAAAAAUC | |
| S264- | AAACGCUUCCUAUAACUC | 264 | UAAACUCGAGUUAUAGGA | 554 |
| AS554 | GAGUUTA | | AGCGUUUCA | |
| S265- | UAUAGGGGAAGAAAAAG | 265 | AACAAUAGCUUUUUCUUC | 555 |
| AS555 | CUAUUGTT | | CCCUAUAAA | |
| S266- | AUAGGGGAAGAAAAAGC | 266 | AAACAAUAGCUUUUUCUU | 556 |
| AS556 | UAUUGUTT | | CCCCUAUAA | |
| S267- | GGGGAAGAAAAAGCUAU | 267 | UGUAAACAAUAGCUUUUU | 557 |
| AS557 | UGUUUACA | | CUUCCCCUA | |
| S268- | GGGAAGAAAAAGCUAUU | 268 | UUGUAAACAAUAGCUUUU | 558 |
| AS558 | GUUUACAA | | UCUUCCCCU | |
| S269- | GGAAGAAAAAGCUAUUG | 269 | AUUGUAAACAAUAGCUUU | 559 |
| AS559 | UUUACAAT | | UUCUUCCCC | |
| S270- | GAAGAAAAAGCUAUUGU | 270 | AAUUGUAAACAAUAGCUU | 560 |
| AS560 | UUACAATT | | UUUCUUCCC | |
| S271- | AAGAAAAAGCUAUUGUU | 271 | UAAUUGUAAACAAUAGCU | 561 |
| AS561 | UACAAUTA | | UUUUCUUCC | |
| S272- | AGAAAAAGCUAUUGUUU | 272 | AUAAUUGUAAACAAUAGC | 562 |
| AS562 | ACAAUUAT | | UUUUUCUUC | |
| S273- | GAAAAAGCUAUUGUUUAC | 273 | UAUAAUUGUAAACAAUAG | 563 |
| AS563 | AAUUATA | | CUUUUUCUU | |
| S274- | AAAAAGCUAUUGUUUACA | 274 | AUAUAAUUGUAAACAAUA | 564 |
| AS564 | AUUAUAT | | GCUUUUUCU | |
| S275- | AAAAGCUAUUGUUUACAA | 275 | GAUAUAAUUGUAAACAAU | 565 |
| AS565 | UUAUATC | | AGCUUUUUC | |
| S276- | AAAGCUAUUGUUUACAAU | 276 | UGAUAUAAUUGUAAACAA | 566 |
| AS566 | UAUAUCA | | UAGCUUUUU | |
| S277- | AAGCUAUUGUUUACAAUU | 277 | GUGAUAUAAUUGUAAACA | 567 |
| AS567 | AUAUCAC | | AUAGCUUUU | |
| S278- | AGCUAUUGUUUACAAUUA | 278 | GGUGAUAUAAUUGUAAAC | 568 |
| AS568 | UAUCACC | | AAUAGCUUU | |
| S279- | GCUAUUGUUUACAAUUAU | 279 | UGGUGAUAUAAUUGUAAA | 569 |
| AS569 | AUCACCA | | CAAUAGCUU | |
| S280- | CUAUUGUUUACAAUUAUA | 280 | AUGGUGAUAUAAUUGUAA | 570 |
| AS570- | UCACCAT | | ACAAUAGCU | |
| M1 | | | | |
| S281- | UAUUGUUUACAAUUAUA | 281 | AAUGGUGAUAUAAUUGUA | 571 |
| AS571 | UCACCATT | | AACAAUAGC | |
| S282- | AUUGUUUACAAUUAUAUC | 282 | UAAUGGUGAUAUAAUUGU | 572 |
| AS572 | ACCAUTA | | AAACAAUAG | |
| S283- | UUGUUUACAAUUAUAUCA | 283 | UUAAUGGUGAUAUAAUUG | 573 |
| AS573 | CCAUUAA | | UAAACAAUA | |
| S284- | UGUUUACAAUUAUAUCAC | 284 | CUUAAUGGUGAUAUAAUU | 574 |
| AS574 | CAUUAAG | | GUAAACAAU | |
| S285- | GUUUACAAUUAUAUCACC | 285 | CCUUAAUGGUGAUAUAAU | 575 |
| AS575 | AUUAAGG | | UGUAAACAA | |
| S286- | UACAAUUAUAUCACCAUU | 286 | UUGCCUUAAUGGUGAUAU | 576 |
| AS576 | AAGGCAA | | AAUUGUAAA | |
| S287- | AUUAUAUCACCAUUAAGG | 287 | GCAGUUGCCUUAAUGGUG | 577 |
| AS577 | CAACUGC | | AUAUAAUUG | |
| S288- | ACUGCUACACCCUGCUUU | 288 | AGAAUACAAAGCAGGGUG | 578 |
| AS578 | GUAUUCT | | UAGCAGUUG | |
| S289- | CUGCUACACCCUGCUUUG | 289 | CAGAAUACAAAGCAGGGU | 579 |
| AS579 | UAUUCTG | | GUAGCAGUU | |
| S290- | UGCUACACCCUGCUUUGU | 290 | CCAGAAUACAAAGCAGGG | 580 |
| AS580 | AUUCUGG | | UGUAGCAGU | |
| S581- | UUCAUAAACAAUGAAUGG | 581 | UGCCAUUCAUUGUUUAUG | 591 |
| AS591 | CAGCAGCCGAAAGGCUGC | | AAGG | |
| S582- | UCAUAAACAAUGAAUGGC | 582 | UUGCCAUUCAUUGUUUAU | 592 |
| AS592 | AAGCAGCCGAAAGGCUGC | | GAGG | |
| S583- | GAAACGUGGUUGUGAUGA | 583 | CUUCAUCACAACCACGUU | 593 |
| AS593 | AGGCAGCCGAAAGGCUGC | | UCGG | |
| S584- | GUUGUGAUGAAGGUAGCU | 584 | UCAGCUACCUUCAUCACA | 594 |
| AS594 | GAGCAGCCGAAAGGCUGC | | ACGG | |
| S585- | GGUGGAUGAAACUCAGUU | 585 | UAAACUGAGUUUCAUCCA | 595 |
| AS595 | UAGCAGCCGAAAGGCUGC | | CCGG | |
| S586- | CAGUUUAAGAAGAUCCUC | 586 | CCGAGGAUCUUCUUAAAC | 596 |
| AS596 | GGGCAGCCGAAAGGCUGC | | UGGG | |
| S587- | UUUAAGAAGAUCCUCGGC | 587 | UAGCCGAGGAUCUUCUUA | 597 |
| AS597 | UAGCAGCCGAAAGGCUGC | | AAGG | |
| S588- | GUUCUAGAAUUUGAAUUG | 588 | AUCAAUUCAAAUUCUAGA | 598 |
| AS598 | AUGCAGCCGAAAGGCUGC | | ACGG | |
| S589- | CCUUUUAAACGACAACAA | 589 | UAUUGUUGUCGUUUAAAA | 599 |
| AS599 | UAGCAGCCGAAAGGCUGC | | GGGG | |
| S590- | AUGAUUUUUAAAAAAUAG | 590 | AUCUAUUUUUUAAAAAUC | 600 |
| AS600 | AUGCAGCCGAAAGGCUGC | | AUGG | |
| S608- | GAAACUCAGUUUAGCAGC | 608 | UAAACUGAGUUUCAUCCA | 595 |
| AS595 | CGAAAGGCUGC | | CCGG | |
| S609- | GGUGGAUGAAACUCAGUU | 609 | UAAACUGAGUUUCAUCCA | 595 |
| AS595 | UA | | CCGG | |

What is claimed is:

1. An oligonucleotide comprising an antisense strand and a sense strand,
wherein the antisense strand is 21 to 27 nucleotides in length and has a region of complementarity to ALDH2,
wherein the sense strand comprises at its 3′-end a stem-loop set forth as: S₁-L-S₂, wherein S₁ is complementary to S₂, and wherein L is a tetraloop and comprises a sequence set forth as GAAA, wherein the GAAA sequence comprises a structure selected from the group consisting of:
(i) each of the A in GAAA sequence is conjugated to a GalNAc moiety, and the G in the GAAA sequence comprises a 2′-O-methyl modification;
(ii) each of the A in GAAA sequence is conjugated to a GalNAc moiety, and the G in the GAAA sequence comprises a 2′-OH;
(iii) each of the nucleotide in the GAAA sequence comprises a 2′-O-methyl modification;
(iv) each of the A in the GAAA sequence comprises a 2′-OH and the G in the GAAA sequence comprises a 2′-O-methyl modification;
(v) each of the A in the GAAA sequence comprises a 2′-O-methoxyethyl modification and the G in the GAAA sequence comprises a 2′-O-methyl modification; and
(vi) each of the A in the GAAA sequence comprises a 2′-adem modification and the G in the GAAA sequence comprises a 2′-O-methyl modification,
and wherein the antisense strand and the sense strand form a duplex structure of at least 12 nucleotides in length but are not covalently linked.

2. The oligonucleotide of claim 1, wherein the antisense strand comprises a sequence set forth in any one of SEQ ID NOs: 591-600.

3. The oligonucleotide of claim 1 or 2, wherein the sense strand comprises a sequence set forth in any one of SEQ ID NOs: 581-590.

4. A pharmaceutical composition comprising an oligonucleotide of any one of claims 1 to 3, and a pharmaceutically acceptable carrier.

5. A method of reducing expression of ALDH2 in a subject, the method comprising administering to the cerebrospinal fluid of the subject an oligonucleotide comprising an antisense strand of 15 to 30 nucleotides in length, wherein the antisense strand has a region of complementarity to a target sequence of ALDH2 as set forth in any one of SEQ ID NOs: 601-607, wherein the region of complementarity is at least 12 contiguous nucleotides in length.

6. The method of claim 5, wherein the region of complementarity is fully complementary to the target sequence of ALDH2.

7. The method of claim 5 or 6, wherein the antisense strand is 19 to 27 nucleotides in length.

8. The method of any one of claims 5 to 7, wherein the region of complementarity to ALDH2 is at least 13 contiguous nucleotides in length

9. The method of any one of claims 5 to 8, wherein the antisense strand comprises a sequence as set forth in any one of SEQ ID NOs: 591-600.

10. The method of any one of claims 5 to 8, wherein the antisense strand consists of a sequence as set forth in any one of SEQ ID NOs: 591-600.

11. The method of any one of claims 5 to 10, wherein the oligonucleotide comprises at least one modified nucleotide.

12. The method of claim 11, wherein the modified nucleotide comprises a 2′-modification.

13. The method of claim 12, wherein the 2′-modification is a modification selected from: 2′-aminoethyl, 2′-fluoro, 2′-O-methyl, 2′-O-methoxyethyl, 2′-adem, 2′-aminodiethoxymethanol, and 2′-deoxy-2′-fluoro-β-d-arabinonucleic acid.

14. The method of any one of claims 11 to 13, wherein all of the nucleotides of the oligonucleotide are modified.

15. The method of any one of claims 5 to 14, wherein the oligonucleotide comprises at least one modified internucleotide linkage.

16. The method of claim 15, wherein the at least one modified internucleotide linkage is a phosphorothioate linkage.

17. The method of any one of claims 5 to 16, wherein the antisense strand comprises a phosphate analog at the 4′-carbon of the sugar of the 5′-nucleotide.

18. The method of claim 17, wherein the phosphate analog is oxymethylphosphonate, vinylphosphonate, or malonylphosphonate.

19. A method of reducing expression of ALDH2 in a subject, the method comprising administering to the cerebrospinal fluid of the subject an oligonucleotide comprising an antisense strand of 15 to 30 nucleotides in length, and a sense strand of 15 to 40 nucleotides in length, wherein the sense strand forms a duplex region with the antisense strand, and wherein the antisense strand has a region of complementarity to a target sequence of ALDH2 as set forth in any one of SEQ ID NOs: 601-607, wherein the region of complementarity is at least 12 contiguous nucleotides in length.

20. The method of claim 19, wherein the sense strand is 19 to 40 nucleotides in length.

21. The method of claim 19 or 20, wherein the duplex region is at least 12 nucleotides in length.

22. The method of any one of claims 19 to 21, wherein the region of complementarity to ALDH2 is at least 13 contiguous nucleotides in length.

23. The method of claim 19 or 22, wherein the antisense strand is 19 to 27 nucleotides in length.

24. The method of any one of claims 19 to 23, wherein the antisense strand comprises a sequence as set forth in any one of SEQ ID NOs: 591-600.

25. The method of any one of claims 19 to 24, wherein the sense strand comprises a sequence as set forth in any one of SEQ ID NOs: 581-590, 608, and 609.

26. The method of any one of claims 19 to 23, wherein the antisense strand consists of a sequence as set forth in any one of SEQ ID NOs: 591-600.

27. The method of any one of claims 19 to 23 and 26, wherein the sense strand consists of a sequence as set forth in any one of SEQ ID NOs: 581-590, 608, and 609.

28. The method of any one of claims 19 to 27, wherein the sense strand comprises at its 3′-end a stem-loop sequence set forth as: S₁-L-S₂, wherein S₁ is complementary to S₂, and wherein L forms a loop between S₁ and S₂ of 3 to 5 nucleotides in length.

29. The method of claim 28, wherein L is a tetraloop.

30. The method of claim 28 or 29, wherein L is 4 nucleotides in length.

31. The method of any one of claims 28 to 30, wherein L comprises a sequence set forth as GAAA.

32. The method of claim 31, wherein at least one nucleotide in the GAAA sequence is conjugated to a GalNAc moiety.

33. The method of claim 32, wherein each of the A in GAAA sequence is conjugated to a GalNAc moiety.

34. The method of any one of claims 19 to 33, wherein the antisense strand and the sense strand are not covalently linked.

35. The method of any one of claims 19 to 34, wherein the oligonucleotide comprises at least one modified nucleotide.

36. The method of claim 35, wherein the modified nucleotide comprises a 2′-modification.

37. The method of claim 36, wherein the 2′-modification is a modification selected from: 2′-aminoethyl, 2′-fluoro, 2′-O-methyl, 2′-O-methoxyethyl, 2′-adem, 2′-aminodiethoxymethanol, and 2′-deoxy-2′-fluoro-β-d-arabinonucleic acid.

38. The method of any one of claims 35 to 37, wherein all of the nucleotides of the oligonucleotide are modified.

39. The method of any one of claims 19 to 38, wherein the oligonucleotide comprises at least one modified internucleotide linkage.

40. The method of claim 39, wherein the at least one modified internucleotide linkage is a phosphorothioate linkage.

41. The method of any one of claims 19 to 40, wherein the antisense strand comprises a phosphate analog at the 4′-carbon of the sugar of the 5′-nucleotide.

42. The method of claim 41, wherein the phosphate analog is oxymethylphosphonate, vinylphosphonate, or malonylphosphonate.

43. The method of any one of claims 35 to 42, wherein the G in the GAAA sequence of claim 31 comprises a 2′-O-methyl modification.

44. The method of any one of claims 35 to 42, wherein the G in the GAAA sequence of claim 31 comprises a 2′-OH.

45. The method of any one of claims 35 to 42, wherein each of the nucleotides in the GAAA sequence of claim 31 comprises a 2′-O-methyl modification.

46. The method of any one of claims 35 to 42, wherein for the GAAA sequence of claim 31, each of the A in the GAAA sequence comprises a 2′-OH and the G in the GAAA sequence comprises a 2′-O-methyl modification.

47. The method of any one of claims 35 to 42, wherein for the GAAA sequence of claim 31, each of the A in the GAAA sequence comprises a 2′-O-methoxyethyl modification and the G in the GAAA sequence comprises a 2′-O-methyl modification.

48. The method of any one of claims 35 to 42, wherein for the GAAA sequence of claim 31, each of the A in the GAAA sequence comprises a 2′-adem modification and the G in the GAAA sequence comprises a 2′-O-methyl modification.

49. The method of any one of claims 5 to 48, wherein the oligonucleotide is administered intrathecally, intraventricularly, intracavitary, or interstitially.

50. The method of any one of claims 5 to 49, wherein the oligonucleotide is administered via injection or infusion.

51. The method of any one of claims 5 to 50, wherein the subject has a neurological disorder.

52. The method of claim 51, wherein the neurological disorder is selected from: neurodegenerative diseases, cognitive disorders, and anxiety disorders.

53. A method of reducing expression of ALDH2 in a subject, the method comprising administering to the cerebrospinal fluid of the subject an oligonucleotide comprising an antisense strand and a sense strand,
wherein the antisense strand is 21 to 27 nucleotides in length and has a region of complementarity to ALDH2,
wherein the sense strand comprises at its 3′-end a stem-loop set forth as: S₁-L-S₂, wherein S₁ is complementary to S₂, and wherein L forms a loop between S₁ and S₂ of 3 to 5 nucleotides in length,
and wherein the antisense strand and the sense strand form a duplex structure of at least 12 nucleotides in length but are not covalently linked.

54. A method of reducing expression of ALDH2 in a subject, the method comprising administering to the cerebrospinal fluid of the subject an oligonucleotide comprising an antisense strand and a sense strand that are not covalently linked,
wherein the antisense strand comprises a sequence as set forth in SEQ ID NO: 595 and the sense strand comprises a sequence as set forth in SEQ ID NO: 585,
wherein the sense strand comprises at its 3′-end a stem-loop set forth as: S₁-L-S₂, wherein S₁ is complementary to S₂, and wherein L is a tetraloop comprising a sequence set forth as GAAA, and wherein the GAAA sequence comprises a structure selected from the group consisting of:
(i) each of the A in GAAA sequence is conjugated to a GalNAc moiety, and the G in the GAAA sequence comprises a 2′-O-methyl modification;
(ii) each of the A in GAAA sequence is conjugated to a GalNAc moiety, and the G in the GAAA sequence comprises a 2′-OH;
(iii) each of the nucleotide in the GAAA sequence comprises a 2′-O-methyl modification;
(iv) each of the A in the GAAA sequence comprises a 2′-OH and the G in the GAAA sequence comprises a 2′-O-methyl modification;
(v) each of the A in the GAAA sequence comprises a 2′-O-methoxyethyl modification and the G in the GAAA sequence comprises a 2′-O-methyl modification; and
(vi) each of the A in the GAAA sequence comprises a 2′-adem modification and the G in the GAAA sequence comprises a 2′-O-methyl modification.

55. A method of reducing expression of ALDH2 in a subject, the method comprising administering to the cerebrospinal fluid of the subject an oligonucleotide comprising an antisense strand and a sense strand that are not covalently linked,
wherein the antisense strand comprises a sequence as set forth in SEQ ID NO: 595 and the sense strand comprises a sequence as set forth in SEQ ID NO: 609.

56. The method of any one of claims 5 to 55, wherein the oligonucleotide reduces expression of ALDH2 that is detectable in somatosensory cortex, hippocampus, frontal cortex, striatum, hypothalamus, cerebellum, and/or spinal cord.

57. A method of treating a neurological disorder associated with ALDH2 expression, the method comprising administering to the cerebrospinal fluid of a subject in need thereof an oligonucleotide comprising an antisense strand of 15 to 30 nucleotides in length, wherein the antisense strand has a region of complementarity to a target sequence of ALDH2 as set forth in any one of SEQ ID NOs: 601-607, wherein the region of complementarity is at least 12 contiguous nucleotides in length.

58. A method treating a neurological disorder associated with ALDH2 expression, the method comprising administering to the cerebrospinal fluid of a subject in need thereof an oligonucleotide comprising an antisense strand and a sense strand,
wherein the antisense strand is 21 to 27 nucleotides in length and has a region of complementarity to ALDH2,
wherein the sense strand comprises at its 3′-end a stem-loop set forth as: S₁-L-S₂, wherein S₁ is complementary to S₂, and wherein L forms a loop between S₁ and S₂ of 3 to 5 nucleotides in length,
and wherein the antisense strand and the sense strand form a duplex structure of at least 12 nucleotides in length but are not covalently linked.

59. The method of claim 57 or 58, wherein the neurological disorder is a neurodegenerative disease.

60. The method of claim 59, wherein the neurological disorder is an anxiety disorder.

61. The method of any one of claims 57 to 60, wherein the oligonucleotide is administered intrathecally, intraventricularly, intracavitary, or interstitially.

62. The method of any one of claims 57 to 61, wherein the oligonucleotide is administered via injection or infusion.

63. The method of any one of claims 57 to 62, wherein the oligonucleotide reduces expression of ALDH2 that is detectable in somatosensory cortex, hippocampus, frontal cortex, striatum, hypothalamus, cerebellum, and/or spinal cord.

64. A method of reducing expression of a target gene in a subject, the method comprising administering an oligonucleotide to the cerebrospinal fluid of the subject, wherein the oligonucleotide comprises an antisense strand and a sense strand,
wherein the antisense strand is 21 to 27 nucleotides in length and has a region of complementarity to the target gene,
wherein the sense strand comprises at its 3′-end a stem-loop set forth as: S₁-L-S₂, wherein S₁ is complementary to S₂, and wherein L forms a loop between S₁ and S₂ of 3 to 5 nucleotides in length,
and wherein the antisense strand and the sense strand form a duplex structure of at least 12 nucleotides in length but are not covalently linked.

65. The method of claim 64, wherein L is a tetraloop.

66. The method of claim 65, wherein L is 4 nucleotides in length.

67. The method of any one of claims 64 to 66, wherein L comprises a sequence set forth as GAAA.

68. The method of claim 67, wherein the GAAA sequence comprises a structure selected from the following:
(i) each of the A in GAAA sequence is conjugated to a GalNAc moiety;
(ii) the G in the GAAA sequence comprises a 2′-O-methyl modification;
(iii) the G in the GAAA sequence comprises a 2′-OH;
(iv) each of the nucleotide in the GAAA sequence comprises a 2′-O-methyl modification;
(v) each of the A in the GAAA sequence comprises a 2′-OH and the G in the GAAA sequence comprises a 2′-O-methyl modification;
(vi) each of the A in the GAAA sequence comprises a 2′-O-methoxyethyl modification and the G in the GAAA sequence comprises a 2′-O-methyl modification; and
(vii) each of the A in the GAAA sequence comprises a 2′-adem and the G in the GAAA sequence comprises a 2′-O-methyl modification.

69. A method of reducing expression of a target gene of interest in a subject, the method comprising administering to the cerebrospinal fluid of the subject an oligonucleotide comprising an antisense strand of 15 to 30 nucleotides in length, wherein the antisense strand has a region of complementarity to a target sequence of the gene of interest that is expressed in the CNS, wherein the region of complementarity is at least 12 contiguous nucleotides in length.

70. The method of any one of claims 64 to 69, wherein the target gene is selected from the group consisting of ALDH2, Ataxin-1, Ataxin-3, APP, BACE1, DYT1, and SOD1.

71. The method of claim 64 to 70, wherein the oligonucleotide reduces expression of the target gene in somatosensory cortex, hippocampus, frontal cortex, striatum, hypothalamus, cerebellum, and/or spinal cord.

72. The method of any one of claims 64 to 71, wherein the oligonucleotide further comprises elements that are degraded by nucleases outside the CNS such that said nucleotide is no longer capable of reducing expression of a gene of interest in a subject in tissues outside the CNS.

73. The method of claim 72, wherein the oligonucleotide further comprises modifications such that it cannot easily exit the CNS.


SEQUENCE LISTING (612 sequences)

SEQ ID NO: 1 - RNA, 25 nt, Artificial Sequence
  Synthetic Polynucleotide
gaggucuucugcaaccagauuuuca

SEQ ID NO: 2 - DNA, 25 bp, Artificial Sequence
  Synthetic Polynucleotide
aggucuucugcaaccagauuuucat

SEQ ID NO: 3 - RNA, 25 nt, Artificial Sequence
  Synthetic Polynucleotide
gucuucugcaaccagauuuucauaa

SEQ ID NO: 4 - RNA, 25 nt, Artificial Sequence
  Synthetic Polynucleotide
cuucugcaaccagauuuucauaaac

SEQ ID NO: 5 - RNA, 25 nt, Artificial Sequence
  Synthetic Polynucleotide
uucugcaaccagauuuucauaaaca

SEQ ID NO: 6 - RNA, 25 nt, Artificial Sequence
  Synthetic Polynucleotide
ucugcaaccagauuuucauaaacaa

SEQ ID NO: 7 - DNA, 25 bp, Artificial Sequence
  Synthetic Polynucleotide
cugcaaccagauuuucauaaacaat

SEQ ID NO: 8 - DNA, 25 bp, Artificial Sequence
  Synthetic Polynucleotide
ugcaaccagauuuucauaaacaatg

SEQ ID NO: 9 - RNA, 25 nt, Artificial Sequence
  Synthetic Polynucleotide
gcaaccagauuuucauaaacaauga

SEQ ID NO: 10 - RNA, 25 nt, Artificial Sequence
  Synthetic Polynucleotide
caaccagauuuucauaaacaaugaa

SEQ ID NO: 11 - DNA, 25 bp, Artificial Sequence
  Synthetic Polynucleotide
aaccagauuuucauaaacaaugaat

SEQ ID NO: 12 - DNA, 25 bp, Artificial Sequence
  Synthetic Polynucleotide
accagauuuucauaaacaaugaatg

SEQ ID NO: 13 - RNA, 25 nt, Artificial Sequence
  Synthetic Polynucleotide
ccagauuuucauaaacaaugaaugg

SEQ ID NO: 14 - RNA, 25 nt, Artificial Sequence
  Synthetic Polynucleotide
cagauuuucauaaacaaugaauggc

SEQ ID NO: 15 - RNA, 25 nt, Artificial Sequence
  Synthetic Polynucleotide
uucauaaacaaugaauggcaugauu

SEQ ID NO: 16 - RNA, 25 nt, Artificial Sequence
  Synthetic Polynucleotide
ucauaaacaaugaauggcaugauuc

SEQ ID NO: 17 - RNA, 25 nt, Artificial Sequence
  Synthetic Polynucleotide
agauuuucauaaacaaugaauggca

SEQ ID NO: 18 - RNA, 25 nt, Artificial Sequence
  Synthetic Polynucleotide
gauuuucauaaacaaugaauggcac

SEQ ID NO: 19 - RNA, 25 nt, Artificial Sequence
  Synthetic Polynucleotide
gccgucagcaggaaaacauucccca

SEQ ID NO: 20 - RNA, 25 nt, Artificial Sequence
  Synthetic Polynucleotide
ccgucagcaggaaaacauuccccac

SEQ ID NO: 21 - RNA, 25 nt, Artificial Sequence
  Synthetic Polynucleotide
ggccuuggagacccuggacaauggc

SEQ ID NO: 22 - RNA, 25 nt, Artificial Sequence
  Synthetic Polynucleotide
gccuuggagacccuggacaauggca

SEQ ID NO: 23 - RNA, 25 nt, Artificial Sequence
  Synthetic Polynucleotide
ccuuggagacccuggacaauggcaa

SEQ ID NO: 24 - RNA, 25 nt, Artificial Sequence
  Synthetic Polynucleotide
uaccugguggauuuggacauggucc

SEQ ID NO: 25 - DNA, 25 bp, Artificial Sequence
  Synthetic Polynucleotide
accugguggauuuggacauggucct

SEQ ID NO: 26 - DNA, 25 bp, Artificial Sequence
  Synthetic Polynucleotide
ccugguggauuuggacauggucctc

SEQ ID NO: 27 - RNA, 25 nt, Artificial Sequence
  Synthetic Polynucleotide
cugguggauuuggacaugguccuca

SEQ ID NO: 28 - RNA, 25 nt, Artificial Sequence
  Synthetic Polynucleotide
ugguggauuuggacaugguccucaa

SEQ ID NO: 29 - RNA, 25 nt, Artificial Sequence
  Synthetic Polynucleotide
gguggauuuggacaugguccucaaa

SEQ ID NO: 30 - DNA, 25 bp, Artificial Sequence
  Synthetic Polynucleotide
guggauuuggacaugguccucaaat

SEQ ID NO: 31 - DNA, 25 bp, Artificial Sequence
  Synthetic Polynucleotide
uggauuuggacaugguccucaaatg

SEQ ID NO: 32 - DNA, 25 bp, Artificial Sequence
  Synthetic Polynucleotide
gauuuggacaugguccucaaaugtc

SEQ ID NO: 33 - RNA, 25 nt, Artificial Sequence
  Synthetic Polynucleotide
uucccgcuccugaugcaagcaugga

SEQ ID NO: 34 - RNA, 25 nt, Artificial Sequence
  Synthetic Polynucleotide
ucccgcuccugaugcaagcauggaa

SEQ ID NO: 35 - RNA, 25 nt, Artificial Sequence
  Synthetic Polynucleotide
cccgcuccugaugcaagcauggaag

SEQ ID NO: 36 - RNA, 25 nt, Artificial Sequence
  Synthetic Polynucleotide
ccgcuccugaugcaagcauggaagc

SEQ ID NO: 37 - DNA, 25 bp, Artificial Sequence
  Synthetic Polynucleotide
cgcuccugaugcaagcauggaagct

SEQ ID NO: 38 - DNA, 25 bp, Artificial Sequence
  Synthetic Polynucleotide
gcuccugaugcaagcauggaagctg

SEQ ID NO: 39 - RNA, 25 nt, Artificial Sequence
  Synthetic Polynucleotide
cuccugaugcaagcauggaagcugg

SEQ ID NO: 40 - RNA, 25 nt, Artificial Sequence
  Synthetic Polynucleotide
uccugaugcaagcauggaagcuggg

SEQ ID NO: 41 - RNA, 25 nt, Artificial Sequence
  Synthetic Polynucleotide
aacuggaaacgugguugugaugaag

SEQ ID NO: 42 - RNA, 25 nt, Artificial Sequence
  Synthetic Polynucleotide
acuggaaacgugguugugaugaagg

SEQ ID NO: 43 - DNA, 25 bp, Artificial Sequence
  Synthetic Polynucleotide
cuggaaacgugguugugaugaaggt

SEQ ID NO: 44 - DNA, 25 bp, Artificial Sequence
  Synthetic Polynucleotide
uggaaacgugguugugaugaaggta

SEQ ID NO: 45 - RNA, 25 nt, Artificial Sequence
  Synthetic Polynucleotide
ggaaacgugguugugaugaagguag

SEQ ID NO: 46 - RNA, 25 nt, Artificial Sequence
  Synthetic Polynucleotide
gaaacgugguugugaugaagguagc

SEQ ID NO: 47 - DNA, 25 bp, Artificial Sequence
  Synthetic Polynucleotide
aacgugguugugaugaagguagctg

SEQ ID NO: 48 - RNA, 25 nt, Artificial Sequence
  Synthetic Polynucleotide
acgugguugugaugaagguagcuga

SEQ ID NO: 49 - RNA, 25 nt, Artificial Sequence
  Synthetic Polynucleotide
cgugguugugaugaagguagcugag

SEQ ID NO: 50 - RNA, 25 nt, Artificial Sequence
  Synthetic Polynucleotide
guugugaugaagguagcugagcaga

SEQ ID NO: 51 - RNA, 25 nt, Artificial Sequence
  Synthetic Polynucleotide
gugaugaagguagcugagcagacac

SEQ ID NO: 52 - RNA, 25 nt, Artificial Sequence
  Synthetic Polynucleotide
aggauguggacaaaguggcauucac

SEQ ID NO: 53 - RNA, 25 nt, Artificial Sequence
  Synthetic Polynucleotide
gggagcagcaaccucaagagaguga

SEQ ID NO: 54 - RNA, 25 nt, Artificial Sequence
  Synthetic Polynucleotide
ggagcagcaaccucaagagagugac

SEQ ID NO: 55 - RNA, 25 nt, Artificial Sequence
  Synthetic Polynucleotide
gagcagcaaccucaagagagugacc

SEQ ID NO: 56 - DNA, 25 bp, Artificial Sequence
  Synthetic Polynucleotide
agcagcaaccucaagagagugacct

SEQ ID NO: 57 - DNA, 25 bp, Artificial Sequence
  Synthetic Polynucleotide
gcagcaaccucaagagagugacctt

SEQ ID NO: 58 - DNA, 25 bp, Artificial Sequence
  Synthetic Polynucleotide
gcccuguucuucaaccagggccagt

SEQ ID NO: 59 - DNA, 25 bp, Artificial Sequence
  Synthetic Polynucleotide
cccuguucuucaaccagggccagtg

SEQ ID NO: 60 - RNA, 25 nt, Artificial Sequence
  Synthetic Polynucleotide
ccuguucuucaaccagggccagugc

SEQ ID NO: 61 - DNA, 25 bp, Artificial Sequence
  Synthetic Polynucleotide
cuguucuucaaccagggccagugct

SEQ ID NO: 62 - DNA, 25 bp, Artificial Sequence
  Synthetic Polynucleotide
uguucuucaaccagggccagugctg

SEQ ID NO: 63 - RNA, 25 nt, Artificial Sequence
  Synthetic Polynucleotide
guucuucaaccagggccagugcugc

SEQ ID NO: 64 - DNA, 25 bp, Artificial Sequence
  Synthetic Polynucleotide
uucuucaaccagggccagugcugct

SEQ ID NO: 65 - DNA, 25 bp, Artificial Sequence
  Synthetic Polynucleotide
cuucaaccagggccagugcugcugt

SEQ ID NO: 66 - DNA, 25 bp, Artificial Sequence
  Synthetic Polynucleotide
uucaaccagggccagugcugcugtg

SEQ ID NO: 67 - RNA, 25 nt, Artificial Sequence
  Synthetic Polynucleotide
caaccagggccagugcugcugugcc

SEQ ID NO: 68 - RNA, 25 nt, Artificial Sequence
  Synthetic Polynucleotide
ggcucccggaccuucgugcaggagg

SEQ ID NO: 69 - RNA, 25 nt, Artificial Sequence
  Synthetic Polynucleotide
gcucccggaccuucgugcaggagga

SEQ ID NO: 70 - RNA, 25 nt, Artificial Sequence
  Synthetic Polynucleotide
cucccggaccuucgugcaggaggac

SEQ ID NO: 71 - RNA, 25 nt, Artificial Sequence
  Synthetic Polynucleotide
ucccggaccuucgugcaggaggaca

SEQ ID NO: 72 - DNA, 25 bp, Artificial Sequence
  Synthetic Polynucleotide
cccggaccuucgugcaggaggacat

SEQ ID NO: 73 - DNA, 25 bp, Artificial Sequence
  Synthetic Polynucleotide
ccggaccuucgugcaggaggacatc

SEQ ID NO: 74 - DNA, 25 bp, Artificial Sequence
  Synthetic Polynucleotide
ggaggacaucuaugaugaguuugtg

SEQ ID NO: 75 - RNA, 25 nt, Artificial Sequence
  Synthetic Polynucleotide
cgggccaagucucggguggucggga

SEQ ID NO: 76 - RNA, 25 nt, Artificial Sequence
  Synthetic Polynucleotide
gggccaagucucggguggucgggaa

SEQ ID NO: 77 - RNA, 25 nt, Artificial Sequence
  Synthetic Polynucleotide
gcagguggaugaaacucaguuuaag

SEQ ID NO: 78 - RNA, 25 nt, Artificial Sequence
  Synthetic Polynucleotide
cagguggaugaaacucaguuuaaga

SEQ ID NO: 79 - RNA, 25 nt, Artificial Sequence
  Synthetic Polynucleotide
agguggaugaaacucaguuuaagaa

SEQ ID NO: 80 - RNA, 25 nt, Artificial Sequence
  Synthetic Polynucleotide
gguggaugaaacucaguuuaagaag

SEQ ID NO: 81 - RNA, 25 nt, Artificial Sequence
  Synthetic Polynucleotide
guggaugaaacucaguuuaagaaga

SEQ ID NO: 82 - DNA, 25 bp, Artificial Sequence
  Synthetic Polynucleotide
uggaugaaacucaguuuaagaagat

SEQ ID NO: 83 - DNA, 25 bp, Artificial Sequence
  Synthetic Polynucleotide
ggaugaaacucaguuuaagaagatc

SEQ ID NO: 84 - RNA, 25 nt, Artificial Sequence
  Synthetic Polynucleotide
gaugaaacucaguuuaagaagaucc

SEQ ID NO: 85 - DNA, 25 bp, Artificial Sequence
  Synthetic Polynucleotide
augaaacucaguuuaagaagaucct

SEQ ID NO: 86 - DNA, 25 bp, Artificial Sequence
  Synthetic Polynucleotide
ugaaacucaguuuaagaagaucctc

SEQ ID NO: 87 - RNA, 25 nt, Artificial Sequence
  Synthetic Polynucleotide
gaaacucaguuuaagaagauccucg

SEQ ID NO: 88 - RNA, 25 nt, Artificial Sequence
  Synthetic Polynucleotide
aaacucaguuuaagaagauccucgg

SEQ ID NO: 89 - RNA, 25 nt, Artificial Sequence
  Synthetic Polynucleotide
aacucaguuuaagaagauccucggc

SEQ ID NO: 90 - DNA, 25 bp, Artificial Sequence
  Synthetic Polynucleotide
acucaguuuaagaagauccucggct

SEQ ID NO: 91 - DNA, 25 bp, Artificial Sequence
  Synthetic Polynucleotide
cucaguuuaagaagauccucggcta

SEQ ID NO: 92 - RNA, 25 nt, Artificial Sequence
  Synthetic Polynucleotide
ucaguuuaagaagauccucggcuac

SEQ ID NO: 93 - RNA, 25 nt, Artificial Sequence
  Synthetic Polynucleotide
caguuuaagaagauccucggcuaca

SEQ ID NO: 94 - DNA, 25 bp, Artificial Sequence
  Synthetic Polynucleotide
aguuuaagaagauccucggcuacat

SEQ ID NO: 95 - DNA, 25 bp, Artificial Sequence
  Synthetic Polynucleotide
guuuaagaagauccucggcuacatc

SEQ ID NO: 96 - RNA, 25 nt, Artificial Sequence
  Synthetic Polynucleotide
uuuaagaagauccucggcuacauca

SEQ ID NO: 97 - RNA, 25 nt, Artificial Sequence
  Synthetic Polynucleotide
uuaagaagauccucggcuacaucaa

SEQ ID NO: 98 - RNA, 25 nt, Artificial Sequence
  Synthetic Polynucleotide
uaagaagauccucggcuacaucaac

SEQ ID NO: 99 - RNA, 25 nt, Artificial Sequence
  Synthetic Polynucleotide
aagaagauccucggcuacaucaaca

SEQ ID NO: 100 - RNA, 25 nt, Artificial Sequence
  Synthetic Polynucleotide
agaagauccucggcuacaucaacac

SEQ ID NO: 101 - RNA, 25 nt, Artificial Sequence
  Synthetic Polynucleotide
gaagauccucggcuacaucaacacg

SEQ ID NO: 102 - RNA, 25 nt, Artificial Sequence
  Synthetic Polynucleotide
aagauccucggcuacaucaacacgg

SEQ ID NO: 103 - RNA, 25 nt, Artificial Sequence
  Synthetic Polynucleotide
agauccucggcuacaucaacacggg

SEQ ID NO: 104 - DNA, 25 bp, Artificial Sequence
  Synthetic Polynucleotide
ugcugcugaccgugguuacuucatc

SEQ ID NO: 105 - RNA, 25 nt, Artificial Sequence
  Synthetic Polynucleotide
gcugcugaccgugguuacuucaucc

SEQ ID NO: 106 - RNA, 25 nt, Artificial Sequence
  Synthetic Polynucleotide
cugcugaccgugguuacuucaucca

SEQ ID NO: 107 - RNA, 25 nt, Artificial Sequence
  Synthetic Polynucleotide
gcugaccgugguuacuucauccagc

SEQ ID NO: 108 - RNA, 25 nt, Artificial Sequence
  Synthetic Polynucleotide
ccagugaugcagauccugaaguuca

SEQ ID NO: 109 - RNA, 25 nt, Artificial Sequence
  Synthetic Polynucleotide
agugaugcagauccugaaguucaag

SEQ ID NO: 110 - RNA, 25 nt, Artificial Sequence
  Synthetic Polynucleotide
gugaugcagauccugaaguucaaga

SEQ ID NO: 111 - RNA, 25 nt, Artificial Sequence
  Synthetic Polynucleotide
ugaugcagauccugaaguucaagac

SEQ ID NO: 112 - RNA, 25 nt, Artificial Sequence
  Synthetic Polynucleotide
gaugcagauccugaaguucaagacc

SEQ ID NO: 113 - RNA, 25 nt, Artificial Sequence
  Synthetic Polynucleotide
augcagauccugaaguucaagacca

SEQ ID NO: 114 - DNA, 25 bp, Artificial Sequence
  Synthetic Polynucleotide
gcagauccugaaguucaagaccata

SEQ ID NO: 115 - RNA, 25 nt, Artificial Sequence
  Synthetic Polynucleotide
cagauccugaaguucaagaccauag

SEQ ID NO: 116 - RNA, 25 nt, Artificial Sequence
  Synthetic Polynucleotide
agauccugaaguucaagaccauaga

SEQ ID NO: 117 - RNA, 25 nt, Artificial Sequence
  Synthetic Polynucleotide
gauccugaaguucaagaccauagag

SEQ ID NO: 118 - RNA, 25 nt, Artificial Sequence
  Synthetic Polynucleotide
uccugaaguucaagaccauagagga

SEQ ID NO: 119 - DNA, 25 bp, Artificial Sequence
  Synthetic Polynucleotide
aaguucaagaccauagaggaggutg

SEQ ID NO: 120 - RNA, 25 nt, Artificial Sequence
  Synthetic Polynucleotide
gcugucuucacaaaggauuuggaca

SEQ ID NO: 121 - RNA, 25 nt, Artificial Sequence
  Synthetic Polynucleotide
gucuucacaaaggauuuggacaagg

SEQ ID NO: 122 - DNA, 25 bp, Artificial Sequence
  Synthetic Polynucleotide
gcaggcauacacugaagugaaaact

SEQ ID NO: 123 - DNA, 25 bp, Artificial Sequence
  Synthetic Polynucleotide
caggcauacacugaagugaaaactg

SEQ ID NO: 124 - DNA, 25 bp, Artificial Sequence
  Synthetic Polynucleotide
aggcauacacugaagugaaaacugt

SEQ ID NO: 125 - DNA, 25 bp, Artificial Sequence
  Synthetic Polynucleotide
ggcauacacugaagugaaaacugtc

SEQ ID NO: 126 - RNA, 25 nt, Artificial Sequence
  Synthetic Polynucleotide
gcauacacugaagugaaaacuguca

SEQ ID NO: 127 - RNA, 25 nt, Artificial Sequence
  Synthetic Polynucleotide
auacacugaagugaaaacugucaca

SEQ ID NO: 128 - RNA, 25 nt, Artificial Sequence
  Synthetic Polynucleotide
uacacugaagugaaaacugucacag

SEQ ID NO: 129 - RNA, 25 nt, Artificial Sequence
  Synthetic Polynucleotide
cugaagugaaaacugucacagucaa

SEQ ID NO: 130 - DNA, 25 bp, Artificial Sequence
  Synthetic Polynucleotide
gucaaagugccucagaagaacucat

SEQ ID NO: 131 - RNA, 25 nt, Artificial Sequence
  Synthetic Polynucleotide
caaagugccucagaagaacucauaa

SEQ ID NO: 132 - RNA, 25 nt, Artificial Sequence
  Synthetic Polynucleotide
aagugccucagaagaacucauaaga

SEQ ID NO: 133 - RNA, 25 nt, Artificial Sequence
  Synthetic Polynucleotide
agugccucagaagaacucauaagaa

SEQ ID NO: 134 - DNA, 25 bp, Artificial Sequence
  Synthetic Polynucleotide
gugccucagaagaacucauaagaat

SEQ ID NO: 135 - DNA, 25 bp, Artificial Sequence
  Synthetic Polynucleotide
ugccucagaagaacucauaagaatc

SEQ ID NO: 136 - DNA, 25 bp, Artificial Sequence
  Synthetic Polynucleotide
ccucagaagaacucauaagaaucat

SEQ ID NO: 137 - DNA, 25 bp, Artificial Sequence
  Synthetic Polynucleotide
cucagaagaacucauaagaaucatg

SEQ ID NO: 138 - RNA, 25 nt, Artificial Sequence
  Synthetic Polynucleotide
ucagaagaacucauaagaaucaugc

SEQ ID NO: 139 - RNA, 25 nt, Artificial Sequence
  Synthetic Polynucleotide
cagaagaacucauaagaaucaugca

SEQ ID NO: 140 - RNA, 25 nt, Artificial Sequence
  Synthetic Polynucleotide
agaagaacucauaagaaucaugcaa

SEQ ID NO: 141 - RNA, 25 nt, Artificial Sequence
  Synthetic Polynucleotide
gaagaacucauaagaaucaugcaag

SEQ ID NO: 142 - RNA, 25 nt, Artificial Sequence
  Synthetic Polynucleotide
aagaacucauaagaaucaugcaagc

SEQ ID NO: 143 - DNA, 25 bp, Artificial Sequence
  Synthetic Polynucleotide
gaacucauaagaaucaugcaagctt

SEQ ID NO: 144 - DNA, 25 bp, Artificial Sequence
  Synthetic Polynucleotide
aacucauaagaaucaugcaagcutc

SEQ ID NO: 145 - RNA, 25 nt, Artificial Sequence
  Synthetic Polynucleotide
cccucagccauugauggaaaguuca

SEQ ID NO: 146 - RNA, 25 nt, Artificial Sequence
  Synthetic Polynucleotide
ccucagccauugauggaaaguucag

SEQ ID NO: 147 - RNA, 25 nt, Artificial Sequence
  Synthetic Polynucleotide
ucagccauugauggaaaguucagca

SEQ ID NO: 148 - RNA, 25 nt, Artificial Sequence
  Synthetic Polynucleotide
cagccauugauggaaaguucagcaa

SEQ ID NO: 149 - RNA, 25 nt, Artificial Sequence
  Synthetic Polynucleotide
agccauugauggaaaguucagcaag

SEQ ID NO: 150 - RNA, 25 nt, Artificial Sequence
  Synthetic Polynucleotide
gccauugauggaaaguucagcaaga

SEQ ID NO: 151 - DNA, 25 bp, Artificial Sequence
  Synthetic Polynucleotide
ccauugauggaaaguucagcaagat

SEQ ID NO: 152 - DNA, 25 bp, Artificial Sequence
  Synthetic Polynucleotide
cauugauggaaaguucagcaagatc

SEQ ID NO: 153 - RNA, 25 nt, Artificial Sequence
  Synthetic Polynucleotide
auugauggaaaguucagcaagauca

SEQ ID NO: 154 - RNA, 25 nt, Artificial Sequence
  Synthetic Polynucleotide
uugauggaaaguucagcaagaucag

SEQ ID NO: 155 - RNA, 25 nt, Artificial Sequence
  Synthetic Polynucleotide
ugauggaaaguucagcaagaucagc

SEQ ID NO: 156 - RNA, 25 nt, Artificial Sequence
  Synthetic Polynucleotide
gauggaaaguucagcaagaucagca

SEQ ID NO: 157 - RNA, 25 nt, Artificial Sequence
  Synthetic Polynucleotide
auggaaaguucagcaagaucagcaa

SEQ ID NO: 158 - RNA, 25 nt, Artificial Sequence
  Synthetic Polynucleotide
uggaaaguucagcaagaucagcaac

SEQ ID NO: 159 - RNA, 25 nt, Artificial Sequence
  Synthetic Polynucleotide
ggaaaguucagcaagaucagcaaca

SEQ ID NO: 160 - RNA, 25 nt, Artificial Sequence
  Synthetic Polynucleotide
gaaaguucagcaagaucagcaacaa

SEQ ID NO: 161 - RNA, 25 nt, Artificial Sequence
  Synthetic Polynucleotide
aaaguucagcaagaucagcaacaaa

SEQ ID NO: 162 - RNA, 25 nt, Artificial Sequence
  Synthetic Polynucleotide
aaguucagcaagaucagcaacaaaa

SEQ ID NO: 163 - DNA, 25 bp, Artificial Sequence
  Synthetic Polynucleotide
aucagcaacaaaaccaagaaaaatg

SEQ ID NO: 164 - DNA, 25 bp, Artificial Sequence
  Synthetic Polynucleotide
cagcaacaaaaccaagaaaaaugat

SEQ ID NO: 165 - DNA, 25 bp, Artificial Sequence
  Synthetic Polynucleotide
agcaacaaaaccaagaaaaaugatc

SEQ ID NO: 166 - DNA, 25 bp, Artificial Sequence
  Synthetic Polynucleotide
acaaaaccaagaaaaaugauccutg

SEQ ID NO: 167 - RNA, 25 nt, Artificial Sequence
  Synthetic Polynucleotide
caaaaccaagaaaaaugauccuugc

SEQ ID NO: 168 - RNA, 25 nt, Artificial Sequence
  Synthetic Polynucleotide
agaaaaaugauccuugcgugcugaa

SEQ ID NO: 169 - DNA, 25 bp, Artificial Sequence
  Synthetic Polynucleotide
aaaaaugauccuugcgugcugaata

SEQ ID NO: 170 - DNA, 25 bp, Artificial Sequence
  Synthetic Polynucleotide
aaaaugauccuugcgugcugaauat

SEQ ID NO: 171 - DNA, 25 bp, Artificial Sequence
  Synthetic Polynucleotide
aaaugauccuugcgugcugaauatc

SEQ ID NO: 172 - DNA, 25 bp, Artificial Sequence
  Synthetic Polynucleotide
aaugauccuugcgugcugaauauct

SEQ ID NO: 173 - DNA, 25 bp, Artificial Sequence
  Synthetic Polynucleotide
augauccuugcgugcugaauauctg

SEQ ID NO: 174 - RNA, 25 nt, Artificial Sequence
  Synthetic Polynucleotide
ugauccuugcgugcugaauaucuga

SEQ ID NO: 175 - RNA, 25 nt, Artificial Sequence
  Synthetic Polynucleotide
gauccuugcgugcugaauaucugaa

SEQ ID NO: 176 - RNA, 25 nt, Artificial Sequence
  Synthetic Polynucleotide
uccuugcgugcugaauaucugaaaa

SEQ ID NO: 177 - RNA, 25 nt, Artificial Sequence
  Synthetic Polynucleotide
ccuugcgugcugaauaucugaaaag

SEQ ID NO: 178 - RNA, 25 nt, Artificial Sequence
  Synthetic Polynucleotide
cuugcgugcugaauaucugaaaaga

SEQ ID NO: 179 - RNA, 25 nt, Artificial Sequence
  Synthetic Polynucleotide
uugcgugcugaauaucugaaaagag

SEQ ID NO: 180 - RNA, 25 nt, Artificial Sequence
  Synthetic Polynucleotide
ugcgugcugaauaucugaaaagaga

SEQ ID NO: 181 - RNA, 25 nt, Artificial Sequence
  Synthetic Polynucleotide
gcgugcugaauaucugaaaagagaa

SEQ ID NO: 182 - RNA, 25 nt, Artificial Sequence
  Synthetic Polynucleotide
cgugcugaauaucugaaaagagaaa

SEQ ID NO: 183 - DNA, 25 bp, Artificial Sequence
  Synthetic Polynucleotide
gugcugaauaucugaaaagagaaat

SEQ ID NO: 184 - DNA, 25 bp, Artificial Sequence
  Synthetic Polynucleotide
ugcugaauaucugaaaagagaaatt

SEQ ID NO: 185 - DNA, 25 bp, Artificial Sequence
  Synthetic Polynucleotide
gcugaauaucugaaaagagaaautt

SEQ ID NO: 186 - DNA, 25 bp, Artificial Sequence
  Synthetic Polynucleotide
cugaauaucugaaaagagaaauutt

SEQ ID NO: 187 - DNA, 25 bp, Artificial Sequence
  Synthetic Polynucleotide
ugaauaucugaaaagagaaauuutt

SEQ ID NO: 188 - DNA, 25 bp, Artificial Sequence
  Synthetic Polynucleotide
gaauaucugaaaagagaaauuuutc

SEQ ID NO: 189 - RNA, 25 nt, Artificial Sequence
  Synthetic Polynucleotide
aauaucugaaaagagaaauuuuucc

SEQ ID NO: 190 - DNA, 25 bp, Artificial Sequence
  Synthetic Polynucleotide
auaucugaaaagagaaauuuuucct

SEQ ID NO: 191 - RNA, 25 nt, Artificial Sequence
  Synthetic Polynucleotide
aucugaaaagagaaauuuuuccuac

SEQ ID NO: 192 - RNA, 25 nt, Artificial Sequence
  Synthetic Polynucleotide
gaaaagagaaauuuuuccuacaaaa

SEQ ID NO: 193 - DNA, 25 bp, Artificial Sequence
  Synthetic Polynucleotide
aaaagagaaauuuuuccuacaaaat

SEQ ID NO: 194 - DNA, 25 bp, Artificial Sequence
  Synthetic Polynucleotide
agagaaauuuuuccuacaaaauctc

SEQ ID NO: 195 - DNA, 25 bp, Artificial Sequence
  Synthetic Polynucleotide
gagaaauuuuuccuacaaaaucuct

SEQ ID NO: 196 - DNA, 25 bp, Artificial Sequence
  Synthetic Polynucleotide
agaaauuuuuccuacaaaaucuctt

SEQ ID NO: 197 - DNA, 25 bp, Artificial Sequence
  Synthetic Polynucleotide
cuugggucaagaaaguucuagaatt

SEQ ID NO: 198 - RNA, 25 nt, Artificial Sequence
  Synthetic Polynucleotide
gggucaagaaaguucuagaauuuga

SEQ ID NO: 199 - RNA, 25 nt, Artificial Sequence
  Synthetic Polynucleotide
ggucaagaaaguucuagaauuugaa

SEQ ID NO: 200 - DNA, 25 bp, Artificial Sequence
  Synthetic Polynucleotide
gucaagaaaguucuagaauuugaat

SEQ ID NO: 201 - DNA, 25 bp, Artificial Sequence
  Synthetic Polynucleotide
ucaagaaaguucuagaauuugaatt

SEQ ID NO: 202 - DNA, 25 bp, Artificial Sequence
  Synthetic Polynucleotide
caagaaaguucuagaauuugaautg

SEQ ID NO: 203 - RNA, 25 nt, Artificial Sequence
  Synthetic Polynucleotide
aagaaaguucuagaauuugaauuga

SEQ ID NO: 204 - DNA, 25 bp, Artificial Sequence
  Synthetic Polynucleotide
agaaaguucuagaauuugaauugat

SEQ ID NO: 205 - DNA, 25 bp, Artificial Sequence
  Synthetic Polynucleotide
gaaaguucuagaauuugaauugata

SEQ ID NO: 206 - RNA, 25 nt, Artificial Sequence
  Synthetic Polynucleotide
aaaguucuagaauuugaauugauaa

SEQ ID NO: 207 - RNA, 25 nt, Artificial Sequence
  Synthetic Polynucleotide
aaguucuagaauuugaauugauaaa

SEQ ID NO: 208 - RNA, 25 nt, Artificial Sequence
  Synthetic Polynucleotide
aguucuagaauuugaauugauaaac

SEQ ID NO: 209 - RNA, 25 nt, Artificial Sequence
  Synthetic Polynucleotide
guucuagaauuugaauugauaaaca

SEQ ID NO: 210 - DNA, 25 bp, Artificial Sequence
  Synthetic Polynucleotide
uucuagaauuugaauugauaaacat

SEQ ID NO: 211 - DNA, 25 bp, Artificial Sequence
  Synthetic Polynucleotide
ucuagaauuugaauugauaaacatg

SEQ ID NO: 212 - RNA, 25 nt, Artificial Sequence
  Synthetic Polynucleotide
cuagaauuugaauugauaaacaugg

SEQ ID NO: 213 - DNA, 25 bp, Artificial Sequence
  Synthetic Polynucleotide
uagaauuugaauugauaaacauggt

SEQ ID NO: 214 - DNA, 25 bp, Artificial Sequence
  Synthetic Polynucleotide
agaauuugaauugauaaacauggtg

SEQ ID NO: 215 - RNA, 25 nt, Artificial Sequence
  Synthetic Polynucleotide
gaauuugaauugauaaacauggugg

SEQ ID NO: 216 - RNA, 25 nt, Artificial Sequence
  Synthetic Polynucleotide
uaagaguauaugaggaaccuuuuaa

SEQ ID NO: 217 - RNA, 25 nt, Artificial Sequence
  Synthetic Polynucleotide
aagaguauaugaggaaccuuuuaaa

SEQ ID NO: 218 - RNA, 25 nt, Artificial Sequence
  Synthetic Polynucleotide
agaguauaugaggaaccuuuuaaac

SEQ ID NO: 219 - RNA, 25 nt, Artificial Sequence
  Synthetic Polynucleotide
gaguauaugaggaaccuuuuaaacg

SEQ ID NO: 220 - RNA, 25 nt, Artificial Sequence
  Synthetic Polynucleotide
aguauaugaggaaccuuuuaaacga

SEQ ID NO: 221 - RNA, 25 nt, Artificial Sequence
  Synthetic Polynucleotide
guauaugaggaaccuuuuaaacgac

SEQ ID NO: 222 - RNA, 25 nt, Artificial Sequence
  Synthetic Polynucleotide
uauaugaggaaccuuuuaaacgaca

SEQ ID NO: 223 - RNA, 25 nt, Artificial Sequence
  Synthetic Polynucleotide
augaggaaccuuuuaaacgacaaca

SEQ ID NO: 224 - DNA, 25 bp, Artificial Sequence
  Synthetic Polynucleotide
gaggaaccuuuuaaacgacaacaat

SEQ ID NO: 225 - DNA, 25 bp, Artificial Sequence
  Synthetic Polynucleotide
aggaaccuuuuaaacgacaacaata

SEQ ID NO: 226 - DNA, 25 bp, Artificial Sequence
  Synthetic Polynucleotide
gaaccuuuuaaacgacaacaauact

SEQ ID NO: 227 - DNA, 25 bp, Artificial Sequence
  Synthetic Polynucleotide
aaccuuuuaaacgacaacaauactg

SEQ ID NO: 228 - RNA, 25 nt, Artificial Sequence
  Synthetic Polynucleotide
accuuuuaaacgacaacaauacugc

SEQ ID NO: 229 - DNA, 25 bp, Artificial Sequence
  Synthetic Polynucleotide
ccuuuuaaacgacaacaauacugct

SEQ ID NO: 230 - DNA, 25 bp, Artificial Sequence
  Synthetic Polynucleotide
cuuuuaaacgacaacaauacugcta

SEQ ID NO: 231 - DNA, 25 bp, Artificial Sequence
  Synthetic Polynucleotide
uaaacgacaacaauacugcuagctt

SEQ ID NO: 232 - DNA, 25 bp, Artificial Sequence
  Synthetic Polynucleotide
aaacgacaacaauacugcuagcutt

SEQ ID NO: 233 - DNA, 25 bp, Artificial Sequence
  Synthetic Polynucleotide
aacgacaacaauacugcuagcuutc

SEQ ID NO: 234 - RNA, 25 nt, Artificial Sequence
  Synthetic Polynucleotide
cgacaacaauacugcuagcuuucag

SEQ ID NO: 235 - RNA, 25 nt, Artificial Sequence
  Synthetic Polynucleotide
gacaacaauacugcuagcuuucagg

SEQ ID NO: 236 - RNA, 25 nt, Artificial Sequence
  Synthetic Polynucleotide
acaacaauacugcuagcuuucagga

SEQ ID NO: 237 - DNA, 25 bp, Artificial Sequence
  Synthetic Polynucleotide
caacaauacugcuagcuuucaggat

SEQ ID NO: 238 - DNA, 25 bp, Artificial Sequence
  Synthetic Polynucleotide
aacaauacugcuagcuuucaggatg

SEQ ID NO: 239 - RNA, 25 nt, Artificial Sequence
  Synthetic Polynucleotide
acaauacugcuagcuuucaggauga

SEQ ID NO: 240 - DNA, 25 bp, Artificial Sequence
  Synthetic Polynucleotide
caauacugcuagcuuucaggaugat

SEQ ID NO: 241 - DNA, 25 bp, Artificial Sequence
  Synthetic Polynucleotide
aauacugcuagcuuucaggaugatt

SEQ ID NO: 242 - DNA, 25 bp, Artificial Sequence
  Synthetic Polynucleotide
auacugcuagcuuucaggaugautt

SEQ ID NO: 243 - DNA, 25 bp, Artificial Sequence
  Synthetic Polynucleotide
uacugcuagcuuucaggaugauutt

SEQ ID NO: 244 - DNA, 25 bp, Artificial Sequence
  Synthetic Polynucleotide
acugcuagcuuucaggaugauuutt

SEQ ID NO: 245 - DNA, 25 bp, Artificial Sequence
  Synthetic Polynucleotide
cugcuagcuuucaggaugauuuuta

SEQ ID NO: 246 - RNA, 25 nt, Artificial Sequence
  Synthetic Polynucleotide
ugcuagcuuucaggaugauuuuuaa

SEQ ID NO: 247 - RNA, 25 nt, Artificial Sequence
  Synthetic Polynucleotide
gcuagcuuucaggaugauuuuuaaa

SEQ ID NO: 248 - RNA, 25 nt, Artificial Sequence
  Synthetic Polynucleotide
cuagcuuucaggaugauuuuuaaaa

SEQ ID NO: 249 - RNA, 25 nt, Artificial Sequence
  Synthetic Polynucleotide
agcuuucaggaugauuuuuaaaaaa

SEQ ID NO: 250 - DNA, 25 bp, Artificial Sequence
  Synthetic Polynucleotide
gcuuucaggaugauuuuuaaaaaat

SEQ ID NO: 251 - DNA, 25 bp, Artificial Sequence
  Synthetic Polynucleotide
cuuucaggaugauuuuuaaaaaata

SEQ ID NO: 252 - RNA, 25 nt, Artificial Sequence
  Synthetic Polynucleotide
uuucaggaugauuuuuaaaaaauag

SEQ ID NO: 253 - RNA, 25 nt, Artificial Sequence
  Synthetic Polynucleotide
uucaggaugauuuuuaaaaaauaga

SEQ ID NO: 254 - DNA, 25 bp, Artificial Sequence
  Synthetic Polynucleotide
ucaggaugauuuuuaaaaaauagat

SEQ ID NO: 255 - DNA, 25 bp, Artificial Sequence
  Synthetic Polynucleotide
caggaugauuuuuaaaaaauagatt

SEQ ID NO: 256 - DNA, 25 bp, Artificial Sequence
  Synthetic Polynucleotide
aggaugauuuuuaaaaaauagautc

SEQ ID NO: 257 - RNA, 25 nt, Artificial Sequence
  Synthetic Polynucleotide
ggaugauuuuuaaaaaauagauuca

SEQ ID NO: 258 - RNA, 25 nt, Artificial Sequence
  Synthetic Polynucleotide
gaugauuuuuaaaaaauagauucaa

SEQ ID NO: 259 - RNA, 25 nt, Artificial Sequence
  Synthetic Polynucleotide
augauuuuuaaaaaauagauucaaa

SEQ ID NO: 260 - DNA, 25 bp, Artificial Sequence
  Synthetic Polynucleotide
ugauuuuuaaaaaauagauucaaat

SEQ ID NO: 261 - DNA, 25 bp, Artificial Sequence
  Synthetic Polynucleotide
gauuuuuaaaaaauagauucaaatg

SEQ ID NO: 262 - DNA, 25 bp, Artificial Sequence
  Synthetic Polynucleotide
auuuuuaaaaaauagauucaaaugt

SEQ ID NO: 263 - DNA, 25 bp, Artificial Sequence
  Synthetic Polynucleotide
uuuuuaaaaaauagauucaaaugtg

SEQ ID NO: 264 - DNA, 25 bp, Artificial Sequence
  Synthetic Polynucleotide
aaacgcuuccuauaacucgaguuta

SEQ ID NO: 265 - DNA, 25 bp, Artificial Sequence
  Synthetic Polynucleotide
uauaggggaagaaaaagcuauugtt

SEQ ID NO: 266 - DNA, 25 bp, Artificial Sequence
  Synthetic Polynucleotide
auaggggaagaaaaagcuauugutt

SEQ ID NO: 267 - RNA, 25 nt, Artificial Sequence
  Synthetic Polynucleotide
ggggaagaaaaagcuauuguuuaca

SEQ ID NO: 268 - RNA, 25 nt, Artificial Sequence
  Synthetic Polynucleotide
gggaagaaaaagcuauuguuuacaa

SEQ ID NO: 269 - DNA, 25 bp, Artificial Sequence
  Synthetic Polynucleotide
ggaagaaaaagcuauuguuuacaat

SEQ ID NO: 270 - DNA, 25 bp, Artificial Sequence
  Synthetic Polynucleotide
gaagaaaaagcuauuguuuacaatt

SEQ ID NO: 271 - DNA, 25 bp, Artificial Sequence
  Synthetic Polynucleotide
aagaaaaagcuauuguuuacaauta

SEQ ID NO: 272 - DNA, 25 bp, Artificial Sequence
  Synthetic Polynucleotide
agaaaaagcuauuguuuacaauuat

SEQ ID NO: 273 - DNA, 25 bp, Artificial Sequence
  Synthetic Polynucleotide
gaaaaagcuauuguuuacaauuata

SEQ ID NO: 274 - DNA, 25 bp, Artificial Sequence
  Synthetic Polynucleotide
aaaaagcuauuguuuacaauuauat

SEQ ID NO: 275 - DNA, 25 bp, Artificial Sequence
  Synthetic Polynucleotide
aaaagcuauuguuuacaauuauatc

SEQ ID NO: 276 - RNA, 25 nt, Artificial Sequence
  Synthetic Polynucleotide
aaagcuauuguuuacaauuauauca

SEQ ID NO: 277 - RNA, 25 nt, Artificial Sequence
  Synthetic Polynucleotide
aagcuauuguuuacaauuauaucac

SEQ ID NO: 278 - RNA, 25 nt, Artificial Sequence
  Synthetic Polynucleotide
agcuauuguuuacaauuauaucacc

SEQ ID NO: 279 - RNA, 25 nt, Artificial Sequence
  Synthetic Polynucleotide
gcuauuguuuacaauuauaucacca

SEQ ID NO: 280 - DNA, 25 bp, Artificial Sequence
  Synthetic Polynucleotide
cuauuguuuacaauuauaucaccat

SEQ ID NO: 281 - DNA, 25 bp, Artificial Sequence
  Synthetic Polynucleotide
uauuguuuacaauuauaucaccatt

SEQ ID NO: 282 - DNA, 25 bp, Artificial Sequence
  Synthetic Polynucleotide
auuguuuacaauuauaucaccauta

SEQ ID NO: 283 - RNA, 25 nt, Artificial Sequence
  Synthetic Polynucleotide
uuguuuacaauuauaucaccauuaa

SEQ ID NO: 284 - RNA, 25 nt, Artificial Sequence
  Synthetic Polynucleotide
uguuuacaauuauaucaccauuaag

SEQ ID NO: 285 - RNA, 25 nt, Artificial Sequence
  Synthetic Polynucleotide
guuuacaauuauaucaccauuaagg

SEQ ID NO: 286 - RNA, 25 nt, Artificial Sequence
  Synthetic Polynucleotide
uacaauuauaucaccauuaaggcaa

SEQ ID NO: 287 - RNA, 25 nt, Artificial Sequence
  Synthetic Polynucleotide
auuauaucaccauuaaggcaacugc

SEQ ID NO: 288 - DNA, 25 bp, Artificial Sequence
  Synthetic Polynucleotide
acugcuacacccugcuuuguauuct

SEQ ID NO: 289 - DNA, 25 bp, Artificial Sequence
  Synthetic Polynucleotide
cugcuacacccugcuuuguauuctg

SEQ ID NO: 290 - RNA, 25 nt, Artificial Sequence
  Synthetic Polynucleotide
ugcuacacccugcuuuguauucugg

SEQ ID NO: 291 - RNA, 27 nt, Artificial Sequence
  Synthetic Polynucleotide
ugaaaaucugguugcagaagaccucgg

SEQ ID NO: 292 - RNA, 27 nt, Artificial Sequence
  Synthetic Polynucleotide
augaaaaucugguugcagaagaccucg

SEQ ID NO: 293 - RNA, 27 nt, Artificial Sequence
  Synthetic Polynucleotide
uuaugaaaaucugguugcagaagaccu

SEQ ID NO: 294 - RNA, 27 nt, Artificial Sequence
  Synthetic Polynucleotide
guuuaugaaaaucugguugcagaagac

SEQ ID NO: 295 - RNA, 27 nt, Artificial Sequence
  Synthetic Polynucleotide
uguuuaugaaaaucugguugcagaaga

SEQ ID NO: 296 - RNA, 27 nt, Artificial Sequence
  Synthetic Polynucleotide
uuguuuaugaaaaucugguugcagaag

SEQ ID NO: 297 - RNA, 27 nt, Artificial Sequence
  Synthetic Polynucleotide
auuguuuaugaaaaucugguugcagaa

SEQ ID NO: 298 - RNA, 27 nt, Artificial Sequence
  Synthetic Polynucleotide
cauuguuuaugaaaaucugguugcaga

SEQ ID NO: 299 - RNA, 27 nt, Artificial Sequence
  Synthetic Polynucleotide
ucauuguuuaugaaaaucugguugcag

SEQ ID NO: 300 - RNA, 27 nt, Artificial Sequence
  Synthetic Polynucleotide
uucauuguuuaugaaaaucugguugca

SEQ ID NO: 301 - RNA, 27 nt, Artificial Sequence
  Synthetic Polynucleotide
auucauuguuuaugaaaaucugguugc

SEQ ID NO: 302 - RNA, 27 nt, Artificial Sequence
  Synthetic Polynucleotide
cauucauuguuuaugaaaaucugguug

SEQ ID NO: 303 - RNA, 27 nt, Artificial Sequence
  Synthetic Polynucleotide
ccauucauuguuuaugaaaaucugguu

SEQ ID NO: 304 - RNA, 27 nt, Artificial Sequence
  Synthetic Polynucleotide
gccauucauuguuuaugaaaaucuggu

SEQ ID NO: 305 - RNA, 27 nt, Artificial Sequence
  Synthetic Polynucleotide
aaucaugccauucauuguuuaugaaga

SEQ ID NO: 306 - RNA, 27 nt, Artificial Sequence
  Synthetic Polynucleotide
gaaucaugccauucauuguuuaugaag

SEQ ID NO: 307 - RNA, 27 nt, Artificial Sequence
  Synthetic Polynucleotide
ugccauucauuguuuaugaaaaucugg

SEQ ID NO: 308 - RNA, 27 nt, Artificial Sequence
  Synthetic Polynucleotide
gugccauucauuguuuaugaaaaucug

SEQ ID NO: 309 - RNA, 27 nt, Artificial Sequence
  Synthetic Polynucleotide
uggggaauguuuuccugcugacggcau

SEQ ID NO: 310 - RNA, 27 nt, Artificial Sequence
  Synthetic Polynucleotide
guggggaauguuuuccugcugacggca

SEQ ID NO: 311 - RNA, 27 nt, Artificial Sequence
  Synthetic Polynucleotide
gccauuguccagggucuccaaggccgc

SEQ ID NO: 312 - RNA, 27 nt, Artificial Sequence
  Synthetic Polynucleotide
ugccauuguccagggucuccaaggccg

SEQ ID NO: 313 - RNA, 27 nt, Artificial Sequence
  Synthetic Polynucleotide
uugccauuguccagggucuccaaggcc

SEQ ID NO: 314 - RNA, 27 nt, Artificial Sequence
  Synthetic Polynucleotide
ggaccauguccaaauccaccagguagg

SEQ ID NO: 315 - RNA, 27 nt, Artificial Sequence
  Synthetic Polynucleotide
aggaccauguccaaauccaccagguag

SEQ ID NO: 316 - RNA, 27 nt, Artificial Sequence
  Synthetic Polynucleotide
gaggaccauguccaaauccaccaggua

SEQ ID NO: 317 - RNA, 27 nt, Artificial Sequence
  Synthetic Polynucleotide
ugaggaccauguccaaauccaccaggu

SEQ ID NO: 318 - RNA, 27 nt, Artificial Sequence
  Synthetic Polynucleotide
uugaggaccauguccaaauccaccagg

SEQ ID NO: 319 - RNA, 27 nt, Artificial Sequence
  Synthetic Polynucleotide
uuugaggaccauguccaaauccaccag

SEQ ID NO: 320 - RNA, 27 nt, Artificial Sequence
  Synthetic Polynucleotide
auuugaggaccauguccaaauccacca

SEQ ID NO: 321 - RNA, 27 nt, Artificial Sequence
  Synthetic Polynucleotide
cauuugaggaccauguccaaauccacc

SEQ ID NO: 322 - RNA, 27 nt, Artificial Sequence
  Synthetic Polynucleotide
gacauuugaggaccauguccaaaucca

SEQ ID NO: 323 - RNA, 27 nt, Artificial Sequence
  Synthetic Polynucleotide
uccaugcuugcaucaggagcgggaaau

SEQ ID NO: 324 - RNA, 27 nt, Artificial Sequence
  Synthetic Polynucleotide
uuccaugcuugcaucaggagcgggaaa

SEQ ID NO: 325 - RNA, 27 nt, Artificial Sequence
  Synthetic Polynucleotide
cuuccaugcuugcaucaggagcgggaa

SEQ ID NO: 326 - RNA, 27 nt, Artificial Sequence
  Synthetic Polynucleotide
gcuuccaugcuugcaucaggagcggga

SEQ ID NO: 327 - RNA, 27 nt, Artificial Sequence
  Synthetic Polynucleotide
agcuuccaugcuugcaucaggagcggg

SEQ ID NO: 328 - RNA, 27 nt, Artificial Sequence
  Synthetic Polynucleotide
cagcuuccaugcuugcaucaggagcgg

SEQ ID NO: 329 - RNA, 27 nt, Artificial Sequence
  Synthetic Polynucleotide
ccagcuuccaugcuugcaucaggagcg

SEQ ID NO: 330 - RNA, 27 nt, Artificial Sequence
  Synthetic Polynucleotide
cccagcuuccaugcuugcaucaggagc

SEQ ID NO: 331 - RNA, 27 nt, Artificial Sequence
  Synthetic Polynucleotide
cuucaucacaaccacguuuccaguugc

SEQ ID NO: 332 - RNA, 27 nt, Artificial Sequence
  Synthetic Polynucleotide
ccuucaucacaaccacguuuccaguug

SEQ ID NO: 333 - RNA, 27 nt, Artificial Sequence
  Synthetic Polynucleotide
accuucaucacaaccacguuuccaguu

SEQ ID NO: 334 - RNA, 27 nt, Artificial Sequence
  Synthetic Polynucleotide
uaccuucaucacaaccacguuuccagu

SEQ ID NO: 335 - RNA, 27 nt, Artificial Sequence
  Synthetic Polynucleotide
cuaccuucaucacaaccacguuuccag

SEQ ID NO: 336 - RNA, 27 nt, Artificial Sequence
  Synthetic Polynucleotide
gcuaccuucaucacaaccacguuucca

SEQ ID NO: 337 - RNA, 27 nt, Artificial Sequence
  Synthetic Polynucleotide
cagcuaccuucaucacaaccacguuuc

SEQ ID NO: 338 - RNA, 27 nt, Artificial Sequence
  Synthetic Polynucleotide
ucagcuaccuucaucacaaccacguuu

SEQ ID NO: 339 - RNA, 27 nt, Artificial Sequence
  Synthetic Polynucleotide
cucagcuaccuucaucacaaccacguu

SEQ ID NO: 340 - RNA, 27 nt, Artificial Sequence
  Synthetic Polynucleotide
ucugcucagcuaccuucaucacaacca

SEQ ID NO: 341 - RNA, 27 nt, Artificial Sequence
  Synthetic Polynucleotide
gugucugcucagcuaccuucaucacaa

SEQ ID NO: 342 - RNA, 27 nt, Artificial Sequence
  Synthetic Polynucleotide
gugaaugccacuuuguccacauccuca

SEQ ID NO: 343 - RNA, 27 nt, Artificial Sequence
  Synthetic Polynucleotide
ucacucucuugagguugcugcucccag

SEQ ID NO: 344 - RNA, 27 nt, Artificial Sequence
  Synthetic Polynucleotide
gucacucucuugagguugcugcuccca

SEQ ID NO: 345 - RNA, 27 nt, Artificial Sequence
  Synthetic Polynucleotide
ggucacucucuugagguugcugcuccc

SEQ ID NO: 346 - RNA, 27 nt, Artificial Sequence
  Synthetic Polynucleotide
aggucacucucuugagguugcugcucc

SEQ ID NO: 347 - RNA, 27 nt, Artificial Sequence
  Synthetic Polynucleotide
aaggucacucucuugagguugcugcuc

SEQ ID NO: 348 - RNA, 27 nt, Artificial Sequence
  Synthetic Polynucleotide
acuggcccugguugaagaacagggcga

SEQ ID NO: 349 - RNA, 27 nt, Artificial Sequence
  Synthetic Polynucleotide
cacuggcccugguugaagaacagggcg

SEQ ID NO: 350 - RNA, 27 nt, Artificial Sequence
  Synthetic Polynucleotide
gcacuggcccugguugaagaacagggc

SEQ ID NO: 351 - RNA, 27 nt, Artificial Sequence
  Synthetic Polynucleotide
agcacuggcccugguugaagaacaggg

SEQ ID NO: 352 - RNA, 27 nt, Artificial Sequence
  Synthetic Polynucleotide
cagcacuggcccugguugaagaacagg

SEQ ID NO: 353 - RNA, 27 nt, Artificial Sequence
  Synthetic Polynucleotide
gcagcacuggcccugguugaagaacag

SEQ ID NO: 354 - RNA, 27 nt, Artificial Sequence
  Synthetic Polynucleotide
agcagcacuggcccugguugaagaaca

SEQ ID NO: 355 - RNA, 27 nt, Artificial Sequence
  Synthetic Polynucleotide
acagcagcacuggcccugguugaagaa

SEQ ID NO: 356 - RNA, 27 nt, Artificial Sequence
  Synthetic Polynucleotide
cacagcagcacuggcccugguugaaga

SEQ ID NO: 357 - RNA, 27 nt, Artificial Sequence
  Synthetic Polynucleotide
ggcacagcagcacuggcccugguugaa

SEQ ID NO: 358 - RNA, 27 nt, Artificial Sequence
  Synthetic Polynucleotide
ccuccugcacgaagguccgggagccgg

SEQ ID NO: 359 - RNA, 27 nt, Artificial Sequence
  Synthetic Polynucleotide
uccuccugcacgaagguccgggagccg

SEQ ID NO: 360 - RNA, 27 nt, Artificial Sequence
  Synthetic Polynucleotide
guccuccugcacgaagguccgggagcc

SEQ ID NO: 361 - RNA, 27 nt, Artificial Sequence
  Synthetic Polynucleotide
uguccuccugcacgaagguccgggagc

SEQ ID NO: 362 - RNA, 27 nt, Artificial Sequence
  Synthetic Polynucleotide
auguccuccugcacgaagguccgggag

SEQ ID NO: 363 - RNA, 27 nt, Artificial Sequence
  Synthetic Polynucleotide
gauguccuccugcacgaagguccggga

SEQ ID NO: 364 - RNA, 27 nt, Artificial Sequence
  Synthetic Polynucleotide
cacaaacucaucauagauguccuccug

SEQ ID NO: 365 - RNA, 27 nt, Artificial Sequence
  Synthetic Polynucleotide
ucccgaccacccgagacuuggcccggg

SEQ ID NO: 366 - RNA, 27 nt, Artificial Sequence
  Synthetic Polynucleotide
uucccgaccacccgagacuuggcccgg

SEQ ID NO: 367 - RNA, 27 nt, Artificial Sequence
  Synthetic Polynucleotide
cuuaaacugaguuucauccaccugcgg

SEQ ID NO: 368 - RNA, 27 nt, Artificial Sequence
  Synthetic Polynucleotide
ucuuaaacugaguuucauccaccugcg

SEQ ID NO: 369 - RNA, 27 nt, Artificial Sequence
  Synthetic Polynucleotide
uucuuaaacugaguuucauccaccugc

SEQ ID NO: 370 - RNA, 27 nt, Artificial Sequence
  Synthetic Polynucleotide
cuucuuaaacugaguuucauccaccug

SEQ ID NO: 371 - RNA, 27 nt, Artificial Sequence
  Synthetic Polynucleotide
ucuucuuaaacugaguuucauccaccu

SEQ ID NO: 372 - RNA, 27 nt, Artificial Sequence
  Synthetic Polynucleotide
aucuucuuaaacugaguuucauccacc

SEQ ID NO: 373 - RNA, 27 nt, Artificial Sequence
  Synthetic Polynucleotide
gaucuucuuaaacugaguuucauccac

SEQ ID NO: 374 - RNA, 27 nt, Artificial Sequence
  Synthetic Polynucleotide
ggaucuucuuaaacugaguuucaucca

SEQ ID NO: 375 - RNA, 27 nt, Artificial Sequence
  Synthetic Polynucleotide
aggaucuucuuaaacugaguuucaucc

SEQ ID NO: 376 - RNA, 27 nt, Artificial Sequence
  Synthetic Polynucleotide
gaggaucuucuuaaacugaguuucauc

SEQ ID NO: 377 - RNA, 27 nt, Artificial Sequence
  Synthetic Polynucleotide
cgaggaucuucuuaaacugaguuucau

SEQ ID NO: 378 - RNA, 27 nt, Artificial Sequence
  Synthetic Polynucleotide
ccgaggaucuucuuaaacugaguuuca

SEQ ID NO: 379 - RNA, 27 nt, Artificial Sequence
  Synthetic Polynucleotide
gccgaggaucuucuuaaacugaguuuc

SEQ ID NO: 380 - RNA, 27 nt, Artificial Sequence
  Synthetic Polynucleotide
agccgaggaucuucuuaaacugaguuu

SEQ ID NO: 381 - RNA, 27 nt, Artificial Sequence
  Synthetic Polynucleotide
uagccgaggaucuucuuaaacugaguu

SEQ ID NO: 382 - RNA, 27 nt, Artificial Sequence
  Synthetic Polynucleotide
guagccgaggaucuucuuaaacugagu

SEQ ID NO: 383 - RNA, 27 nt, Artificial Sequence
  Synthetic Polynucleotide
uguagccgaggaucuucuuaaacugag

SEQ ID NO: 384 - RNA, 27 nt, Artificial Sequence
  Synthetic Polynucleotide
auguagccgaggaucuucuuaaacuga

SEQ ID NO: 385 - RNA, 27 nt, Artificial Sequence
  Synthetic Polynucleotide
gauguagccgaggaucuucuuaaacug

SEQ ID NO: 386 - RNA, 27 nt, Artificial Sequence
  Synthetic Polynucleotide
ugauguagccgaggaucuucuuaaacu

SEQ ID NO: 387 - RNA, 27 nt, Artificial Sequence
  Synthetic Polynucleotide
uugauguagccgaggaucuucuuaaac

SEQ ID NO: 388 - RNA, 27 nt, Artificial Sequence
  Synthetic Polynucleotide
guugauguagccgaggaucuucuuaaa

SEQ ID NO: 389 - RNA, 27 nt, Artificial Sequence
  Synthetic Polynucleotide
uguugauguagccgaggaucuucuuaa

SEQ ID NO: 390 - RNA, 27 nt, Artificial Sequence
  Synthetic Polynucleotide
guguugauguagccgaggaucuucuua

SEQ ID NO: 391 - RNA, 27 nt, Artificial Sequence
  Synthetic Polynucleotide
cguguugauguagccgaggaucuucuu

SEQ ID NO: 392 - RNA, 27 nt, Artificial Sequence
  Synthetic Polynucleotide
ccguguugauguagccgaggaucuucu

SEQ ID NO: 393 - RNA, 27 nt, Artificial Sequence
  Synthetic Polynucleotide
cccguguugauguagccgaggaucuuc

SEQ ID NO: 394 - RNA, 27 nt, Artificial Sequence
  Synthetic Polynucleotide
gaugaaguaaccacggucagcagcaau

SEQ ID NO: 395 - RNA, 27 nt, Artificial Sequence
  Synthetic Polynucleotide
ggaugaaguaaccacggucagcagcaa

SEQ ID NO: 396 - RNA, 27 nt, Artificial Sequence
  Synthetic Polynucleotide
uggaugaaguaaccacggucagcagca

SEQ ID NO: 397 - RNA, 27 nt, Artificial Sequence
  Synthetic Polynucleotide
gcuggaugaaguaaccacggucagcag

SEQ ID NO: 398 - RNA, 27 nt, Artificial Sequence
  Synthetic Polynucleotide
ugaacuucaggaucugcaucacuggcc

SEQ ID NO: 399 - RNA, 27 nt, Artificial Sequence
  Synthetic Polynucleotide
cuugaacuucaggaucugcaucacugg

SEQ ID NO: 400 - RNA, 27 nt, Artificial Sequence
  Synthetic Polynucleotide
ucuugaacuucaggaucugcaucacug

SEQ ID NO: 401 - RNA, 27 nt, Artificial Sequence
  Synthetic Polynucleotide
gucuugaacuucaggaucugcaucacu

SEQ ID NO: 402 - RNA, 27 nt, Artificial Sequence
  Synthetic Polynucleotide
ggucuugaacuucaggaucugcaucac

SEQ ID NO: 403 - RNA, 27 nt, Artificial Sequence
  Synthetic Polynucleotide
uggucuugaacuucaggaucugcauca

SEQ ID NO: 404 - RNA, 27 nt, Artificial Sequence
  Synthetic Polynucleotide
uauggucuugaacuucaggaucugcau

SEQ ID NO: 405 - RNA, 27 nt, Artificial Sequence
  Synthetic Polynucleotide
cuauggucuugaacuucaggaucugca

SEQ ID NO: 406 - RNA, 27 nt, Artificial Sequence
  Synthetic Polynucleotide
ucuauggucuugaacuucaggaucugc

SEQ ID NO: 407 - RNA, 27 nt, Artificial Sequence
  Synthetic Polynucleotide
cucuauggucuugaacuucaggaucug

SEQ ID NO: 408 - RNA, 27 nt, Artificial Sequence
  Synthetic Polynucleotide
uccucuauggucuugaacuucaggauc

SEQ ID NO: 409 - RNA, 27 nt, Artificial Sequence
  Synthetic Polynucleotide
caaccuccucuauggucuugaacuuca

SEQ ID NO: 410 - RNA, 27 nt, Artificial Sequence
  Synthetic Polynucleotide
uguccaaauccuuugugaagacagcug

SEQ ID NO: 411 - RNA, 27 nt, Artificial Sequence
  Synthetic Polynucleotide
ccuuguccaaauccuuugugaagacag

SEQ ID NO: 412 - RNA, 27 nt, Artificial Sequence
  Synthetic Polynucleotide
aguuuucacuucaguguaugccugcag

SEQ ID NO: 413 - RNA, 27 nt, Artificial Sequence
  Synthetic Polynucleotide
caguuuucacuucaguguaugccugca

SEQ ID NO: 414 - RNA, 27 nt, Artificial Sequence
  Synthetic Polynucleotide
acaguuuucacuucaguguaugccugc

SEQ ID NO: 415 - RNA, 27 nt, Artificial Sequence
  Synthetic Polynucleotide
gacaguuuucacuucaguguaugccug

SEQ ID NO: 416 - RNA, 27 nt, Artificial Sequence
  Synthetic Polynucleotide
ugacaguuuucacuucaguguaugccu

SEQ ID NO: 417 - RNA, 27 nt, Artificial Sequence
  Synthetic Polynucleotide
ugugacaguuuucacuucaguguaugc

SEQ ID NO: 418 - RNA, 27 nt, Artificial Sequence
  Synthetic Polynucleotide
cugugacaguuuucacuucaguguaug

SEQ ID NO: 419 - RNA, 27 nt, Artificial Sequence
  Synthetic Polynucleotide
uugacugugacaguuuucacuucagug

SEQ ID NO: 420 - RNA, 27 nt, Artificial Sequence
  Synthetic Polynucleotide
augaguucuucugaggcacuuugacug

SEQ ID NO: 421 - RNA, 27 nt, Artificial Sequence
  Synthetic Polynucleotide
uuaugaguucuucugaggcacuuugac

SEQ ID NO: 422 - RNA, 27 nt, Artificial Sequence
  Synthetic Polynucleotide
ucuuaugaguucuucugaggcacuuug

SEQ ID NO: 423 - RNA, 27 nt, Artificial Sequence
  Synthetic Polynucleotide
uucuuaugaguucuucugaggcacuuu

SEQ ID NO: 424 - RNA, 27 nt, Artificial Sequence
  Synthetic Polynucleotide
auucuuaugaguucuucugaggcacuu

SEQ ID NO: 425 - RNA, 27 nt, Artificial Sequence
  Synthetic Polynucleotide
gauucuuaugaguucuucugaggcacu

SEQ ID NO: 426 - RNA, 27 nt, Artificial Sequence
  Synthetic Polynucleotide
augauucuuaugaguucuucugaggca

SEQ ID NO: 427 - RNA, 27 nt, Artificial Sequence
  Synthetic Polynucleotide
caugauucuuaugaguucuucugaggc

SEQ ID NO: 428 - RNA, 27 nt, Artificial Sequence
  Synthetic Polynucleotide
gcaugauucuuaugaguucuucugagg

SEQ ID NO: 429 - RNA, 27 nt, Artificial Sequence
  Synthetic Polynucleotide
ugcaugauucuuaugaguucuucugag

SEQ ID NO: 430 - RNA, 27 nt, Artificial Sequence
  Synthetic Polynucleotide
uugcaugauucuuaugaguucuucuga

SEQ ID NO: 431 - RNA, 27 nt, Artificial Sequence
  Synthetic Polynucleotide
cuugcaugauucuuaugaguucuucug

SEQ ID NO: 432 - RNA, 27 nt, Artificial Sequence
  Synthetic Polynucleotide
gcuugcaugauucuuaugaguucuucu

SEQ ID NO: 433 - RNA, 27 nt, Artificial Sequence
  Synthetic Polynucleotide
aagcuugcaugauucuuaugaguucuu

SEQ ID NO: 434 - RNA, 27 nt, Artificial Sequence
  Synthetic Polynucleotide
gaagcuugcaugauucuuaugaguucu

SEQ ID NO: 435 - RNA, 27 nt, Artificial Sequence
  Synthetic Polynucleotide
ugaacuuuccaucaauggcugagggag

SEQ ID NO: 436 - RNA, 27 nt, Artificial Sequence
  Synthetic Polynucleotide
cugaacuuuccaucaauggcugaggga

SEQ ID NO: 437 - RNA, 27 nt, Artificial Sequence
  Synthetic Polynucleotide
ugcugaacuuuccaucaauggcugagg

SEQ ID NO: 438 - RNA, 27 nt, Artificial Sequence
  Synthetic Polynucleotide
uugcugaacuuuccaucaauggcugag

SEQ ID NO: 439 - RNA, 27 nt, Artificial Sequence
  Synthetic Polynucleotide
cuugcugaacuuuccaucaauggcuga

SEQ ID NO: 440 - RNA, 27 nt, Artificial Sequence
  Synthetic Polynucleotide
ucuugcugaacuuuccaucaauggcug

SEQ ID NO: 441 - RNA, 27 nt, Artificial Sequence
  Synthetic Polynucleotide
aucuugcugaacuuuccaucaauggcu

SEQ ID NO: 442 - RNA, 27 nt, Artificial Sequence
  Synthetic Polynucleotide
gaucuugcugaacuuuccaucaauggc

SEQ ID NO: 443 - RNA, 27 nt, Artificial Sequence
  Synthetic Polynucleotide
ugaucuugcugaacuuuccaucaaugg

SEQ ID NO: 444 - RNA, 27 nt, Artificial Sequence
  Synthetic Polynucleotide
cugaucuugcugaacuuuccaucaaug

SEQ ID NO: 445 - RNA, 27 nt, Artificial Sequence
  Synthetic Polynucleotide
gcugaucuugcugaacuuuccaucaau

SEQ ID NO: 446 - RNA, 27 nt, Artificial Sequence
  Synthetic Polynucleotide
ugcugaucuugcugaacuuuccaucaa

SEQ ID NO: 447 - RNA, 27 nt, Artificial Sequence
  Synthetic Polynucleotide
uugcugaucuugcugaacuuuccauca

SEQ ID NO: 448 - RNA, 27 nt, Artificial Sequence
  Synthetic Polynucleotide
guugcugaucuugcugaacuuuccauc

SEQ ID NO: 449 - RNA, 27 nt, Artificial Sequence
  Synthetic Polynucleotide
uguugcugaucuugcugaacuuuccau

SEQ ID NO: 450 - RNA, 27 nt, Artificial Sequence
  Synthetic Polynucleotide
uuguugcugaucuugcugaacuuucca

SEQ ID NO: 451 - RNA, 27 nt, Artificial Sequence
  Synthetic Polynucleotide
uuuguugcugaucuugcugaacuuucc

SEQ ID NO: 452 - RNA, 27 nt, Artificial Sequence
  Synthetic Polynucleotide
uuuuguugcugaucuugcugaacuuuc

SEQ ID NO: 453 - RNA, 27 nt, Artificial Sequence
  Synthetic Polynucleotide
cauuuuucuugguuuuguugcugaucu

SEQ ID NO: 454 - RNA, 27 nt, Artificial Sequence
  Synthetic Polynucleotide
aucauuuuucuugguuuuguugcugau

SEQ ID NO: 455 - RNA, 27 nt, Artificial Sequence
  Synthetic Polynucleotide
gaucauuuuucuugguuuuguugcuga

SEQ ID NO: 456 - RNA, 27 nt, Artificial Sequence
  Synthetic Polynucleotide
caaggaucauuuuucuugguuuuguug

SEQ ID NO: 457 - RNA, 27 nt, Artificial Sequence
  Synthetic Polynucleotide
gcaaggaucauuuuucuugguuuuguu

SEQ ID NO: 458 - RNA, 27 nt, Artificial Sequence
  Synthetic Polynucleotide
uucagcacgcaaggaucauuuuucuug

SEQ ID NO: 459 - RNA, 27 nt, Artificial Sequence
  Synthetic Polynucleotide
uauucagcacgcaaggaucauuuuucu

SEQ ID NO: 460 - RNA, 27 nt, Artificial Sequence
  Synthetic Polynucleotide
auauucagcacgcaaggaucauuuuuc

SEQ ID NO: 461 - RNA, 27 nt, Artificial Sequence
  Synthetic Polynucleotide
gauauucagcacgcaaggaucauuuuu

SEQ ID NO: 462 - RNA, 27 nt, Artificial Sequence
  Synthetic Polynucleotide
agauauucagcacgcaaggaucauuuu

SEQ ID NO: 463 - RNA, 27 nt, Artificial Sequence
  Synthetic Polynucleotide
cagauauucagcacgcaaggaucauuu

SEQ ID NO: 464 - RNA, 27 nt, Artificial Sequence
  Synthetic Polynucleotide
ucagauauucagcacgcaaggaucauu

SEQ ID NO: 465 - RNA, 27 nt, Artificial Sequence
  Synthetic Polynucleotide
uucagauauucagcacgcaaggaucau

SEQ ID NO: 466 - RNA, 27 nt, Artificial Sequence
  Synthetic Polynucleotide
uuuucagauauucagcacgcaaggauc

SEQ ID NO: 467 - RNA, 27 nt, Artificial Sequence
  Synthetic Polynucleotide
cuuuucagauauucagcacgcaaggau

SEQ ID NO: 468 - RNA, 27 nt, Artificial Sequence
  Synthetic Polynucleotide
ucuuuucagauauucagcacgcaagga

SEQ ID NO: 469 - RNA, 27 nt, Artificial Sequence
  Synthetic Polynucleotide
cucuuuucagauauucagcacgcaagg

SEQ ID NO: 470 - RNA, 27 nt, Artificial Sequence
  Synthetic Polynucleotide
ucucuuuucagauauucagcacgcaag

SEQ ID NO: 471 - RNA, 27 nt, Artificial Sequence
  Synthetic Polynucleotide
uucucuuuucagauauucagcacgcaa

SEQ ID NO: 472 - RNA, 27 nt, Artificial Sequence
  Synthetic Polynucleotide
uuucucuuuucagauauucagcacgca

SEQ ID NO: 473 - RNA, 27 nt, Artificial Sequence
  Synthetic Polynucleotide
auuucucuuuucagauauucagcacgc

SEQ ID NO: 474 - RNA, 27 nt, Artificial Sequence
  Synthetic Polynucleotide
aauuucucuuuucagauauucagcacg

SEQ ID NO: 475 - RNA, 27 nt, Artificial Sequence
  Synthetic Polynucleotide
aaauuucucuuuucagauauucagcac

SEQ ID NO: 476 - RNA, 27 nt, Artificial Sequence
  Synthetic Polynucleotide
aaaauuucucuuuucagauauucagca

SEQ ID NO: 477 - RNA, 27 nt, Artificial Sequence
  Synthetic Polynucleotide
aaaaauuucucuuuucagauauucagc

SEQ ID NO: 478 - RNA, 27 nt, Artificial Sequence
  Synthetic Polynucleotide
gaaaaauuucucuuuucagauauucag

SEQ ID NO: 479 - RNA, 27 nt, Artificial Sequence
  Synthetic Polynucleotide
ggaaaaauuucucuuuucagauauuca

SEQ ID NO: 480 - RNA, 27 nt, Artificial Sequence
  Synthetic Polynucleotide
aggaaaaauuucucuuuucagauauuc

SEQ ID NO: 481 - RNA, 27 nt, Artificial Sequence
  Synthetic Polynucleotide
guaggaaaaauuucucuuuucagauau

SEQ ID NO: 482 - RNA, 27 nt, Artificial Sequence
  Synthetic Polynucleotide
uuuuguaggaaaaauuucucuuuucag

SEQ ID NO: 483 - RNA, 27 nt, Artificial Sequence
  Synthetic Polynucleotide
auuuuguaggaaaaauuucucuuuuca

SEQ ID NO: 484 - RNA, 27 nt, Artificial Sequence
  Synthetic Polynucleotide
gagauuuuguaggaaaaauuucucuuu

SEQ ID NO: 485 - RNA, 27 nt, Artificial Sequence
  Synthetic Polynucleotide
agagauuuuguaggaaaaauuucucuu

SEQ ID NO: 486 - RNA, 27 nt, Artificial Sequence
  Synthetic Polynucleotide
aagagauuuuguaggaaaaauuucucu

SEQ ID NO: 487 - RNA, 27 nt, Artificial Sequence
  Synthetic Polynucleotide
aauucuagaacuuucuugacccaagag

SEQ ID NO: 488 - RNA, 27 nt, Artificial Sequence
  Synthetic Polynucleotide
ucaaauucuagaacuuucuugacccaa

SEQ ID NO: 489 - RNA, 27 nt, Artificial Sequence
  Synthetic Polynucleotide
uucaaauucuagaacuuucuugaccca

SEQ ID NO: 490 - RNA, 27 nt, Artificial Sequence
  Synthetic Polynucleotide
auucaaauucuagaacuuucuugaccc

SEQ ID NO: 491 - RNA, 27 nt, Artificial Sequence
  Synthetic Polynucleotide
aauucaaauucuagaacuuucuugacc

SEQ ID NO: 492 - RNA, 27 nt, Artificial Sequence
  Synthetic Polynucleotide
caauucaaauucuagaacuuucuugac

SEQ ID NO: 493 - RNA, 27 nt, Artificial Sequence
  Synthetic Polynucleotide
ucaauucaaauucuagaacuuucuuga

SEQ ID NO: 494 - RNA, 27 nt, Artificial Sequence
  Synthetic Polynucleotide
aucaauucaaauucuagaacuuucuug

SEQ ID NO: 495 - RNA, 27 nt, Artificial Sequence
  Synthetic Polynucleotide
uaucaauucaaauucuagaacuuucuu

SEQ ID NO: 496 - RNA, 27 nt, Artificial Sequence
  Synthetic Polynucleotide
uuaucaauucaaauucuagaacuuucu

SEQ ID NO: 497 - RNA, 27 nt, Artificial Sequence
  Synthetic Polynucleotide
uuuaucaauucaaauucuagaacuuuc

SEQ ID NO: 498 - RNA, 27 nt, Artificial Sequence
  Synthetic Polynucleotide
guuuaucaauucaaauucuagaacuuu

SEQ ID NO: 499 - RNA, 27 nt, Artificial Sequence
  Synthetic Polynucleotide
uguuuaucaauucaaauucuagaacuu

SEQ ID NO: 500 - RNA, 27 nt, Artificial Sequence
  Synthetic Polynucleotide
auguuuaucaauucaaauucuagaacu

SEQ ID NO: 501 - RNA, 27 nt, Artificial Sequence
  Synthetic Polynucleotide
cauguuuaucaauucaaauucuagaac

SEQ ID NO: 502 - RNA, 27 nt, Artificial Sequence
  Synthetic Polynucleotide
ccauguuuaucaauucaaauucuagaa

SEQ ID NO: 503 - RNA, 27 nt, Artificial Sequence
  Synthetic Polynucleotide
accauguuuaucaauucaaauucuaga

SEQ ID NO: 504 - RNA, 27 nt, Artificial Sequence
  Synthetic Polynucleotide
caccauguuuaucaauucaaauucuag

SEQ ID NO: 505 - RNA, 27 nt, Artificial Sequence
  Synthetic Polynucleotide
ccaccauguuuaucaauucaaauucua

SEQ ID NO: 506 - RNA, 27 nt, Artificial Sequence
  Synthetic Polynucleotide
uuaaaagguuccucauauacucuuacc

SEQ ID NO: 507 - RNA, 27 nt, Artificial Sequence
  Synthetic Polynucleotide
uuuaaaagguuccucauauacucuuac

SEQ ID NO: 508 - RNA, 27 nt, Artificial Sequence
  Synthetic Polynucleotide
guuuaaaagguuccucauauacucuua

SEQ ID NO: 509 - RNA, 27 nt, Artificial Sequence
  Synthetic Polynucleotide
cguuuaaaagguuccucauauacucuu

SEQ ID NO: 510 - RNA, 27 nt, Artificial Sequence
  Synthetic Polynucleotide
ucguuuaaaagguuccucauauacucu

SEQ ID NO: 511 - RNA, 27 nt, Artificial Sequence
  Synthetic Polynucleotide
gucguuuaaaagguuccucauauacuc

SEQ ID NO: 512 - RNA, 27 nt, Artificial Sequence
  Synthetic Polynucleotide
ugucguuuaaaagguuccucauauacu

SEQ ID NO: 513 - RNA, 27 nt, Artificial Sequence
  Synthetic Polynucleotide
uguugucguuuaaaagguuccucauau

SEQ ID NO: 514 - RNA, 27 nt, Artificial Sequence
  Synthetic Polynucleotide
auuguugucguuuaaaagguuccucau

SEQ ID NO: 515 - RNA, 27 nt, Artificial Sequence
  Synthetic Polynucleotide
uauuguugucguuuaaaagguuccuca

SEQ ID NO: 516 - RNA, 27 nt, Artificial Sequence
  Synthetic Polynucleotide
aguauuguugucguuuaaaagguuccu

SEQ ID NO: 517 - RNA, 27 nt, Artificial Sequence
  Synthetic Polynucleotide
caguauuguugucguuuaaaagguucc

SEQ ID NO: 518 - RNA, 27 nt, Artificial Sequence
  Synthetic Polynucleotide
gcaguauuguugucguuuaaaagguuc

SEQ ID NO: 519 - RNA, 27 nt, Artificial Sequence
  Synthetic Polynucleotide
agcaguauuguugucguuuaaaagguu

SEQ ID NO: 520 - RNA, 27 nt, Artificial Sequence
  Synthetic Polynucleotide
uagcaguauuguugucguuuaaaaggu

SEQ ID NO: 521 - RNA, 27 nt, Artificial Sequence
  Synthetic Polynucleotide
aagcuagcaguauuguugucguuuaaa

SEQ ID NO: 522 - RNA, 27 nt, Artificial Sequence
  Synthetic Polynucleotide
aaagcuagcaguauuguugucguuuaa

SEQ ID NO: 523 - RNA, 27 nt, Artificial Sequence
  Synthetic Polynucleotide
gaaagcuagcaguauuguugucguuua

SEQ ID NO: 524 - RNA, 27 nt, Artificial Sequence
  Synthetic Polynucleotide
cugaaagcuagcaguauuguugucguu

SEQ ID NO: 525 - RNA, 27 nt, Artificial Sequence
  Synthetic Polynucleotide
ccugaaagcuagcaguauuguugucgu

SEQ ID NO: 526 - RNA, 27 nt, Artificial Sequence
  Synthetic Polynucleotide
uccugaaagcuagcaguauuguugucg

SEQ ID NO: 527 - RNA, 27 nt, Artificial Sequence
  Synthetic Polynucleotide
auccugaaagcuagcaguauuguuguc

SEQ ID NO: 528 - RNA, 27 nt, Artificial Sequence
  Synthetic Polynucleotide
cauccugaaagcuagcaguauuguugu

SEQ ID NO: 529 - RNA, 27 nt, Artificial Sequence
  Synthetic Polynucleotide
ucauccugaaagcuagcaguauuguug

SEQ ID NO: 530 - RNA, 27 nt, Artificial Sequence
  Synthetic Polynucleotide
aucauccugaaagcuagcaguauuguu

SEQ ID NO: 531 - RNA, 27 nt, Artificial Sequence
  Synthetic Polynucleotide
aaucauccugaaagcuagcaguauugu

SEQ ID NO: 532 - RNA, 27 nt, Artificial Sequence
  Synthetic Polynucleotide
aaaucauccugaaagcuagcaguauug

SEQ ID NO: 533 - RNA, 27 nt, Artificial Sequence
  Synthetic Polynucleotide
aaaaucauccugaaagcuagcaguauu

SEQ ID NO: 534 - RNA, 27 nt, Artificial Sequence
  Synthetic Polynucleotide
aaaaaucauccugaaagcuagcaguau

SEQ ID NO: 535 - RNA, 27 nt, Artificial Sequence
  Synthetic Polynucleotide
uaaaaaucauccugaaagcuagcagua

SEQ ID NO: 536 - RNA, 27 nt, Artificial Sequence
  Synthetic Polynucleotide
uuaaaaaucauccugaaagcuagcagu

SEQ ID NO: 537 - RNA, 27 nt, Artificial Sequence
  Synthetic Polynucleotide
uuuaaaaaucauccugaaagcuagcag

SEQ ID NO: 538 - RNA, 27 nt, Artificial Sequence
  Synthetic Polynucleotide
uuuuaaaaaucauccugaaagcuagca

SEQ ID NO: 539 - RNA, 27 nt, Artificial Sequence
  Synthetic Polynucleotide
uuuuuuaaaaaucauccugaaagcuag

SEQ ID NO: 540 - RNA, 27 nt, Artificial Sequence
  Synthetic Polynucleotide
auuuuuuaaaaaucauccugaaagcua

SEQ ID NO: 541 - RNA, 27 nt, Artificial Sequence
  Synthetic Polynucleotide
uauuuuuuaaaaaucauccugaaagcu

SEQ ID NO: 542 - RNA, 27 nt, Artificial Sequence
  Synthetic Polynucleotide
cuauuuuuuaaaaaucauccugaaagc

SEQ ID NO: 543 - RNA, 27 nt, Artificial Sequence
  Synthetic Polynucleotide
ucuauuuuuuaaaaaucauccugaaag

SEQ ID NO: 544 - RNA, 27 nt, Artificial Sequence
  Synthetic Polynucleotide
aucuauuuuuuaaaaaucauccugaaa

SEQ ID NO: 545 - RNA, 27 nt, Artificial Sequence
  Synthetic Polynucleotide
aaucuauuuuuuaaaaaucauccugaa

SEQ ID NO: 546 - RNA, 27 nt, Artificial Sequence
  Synthetic Polynucleotide
gaaucuauuuuuuaaaaaucauccuga

SEQ ID NO: 547 - RNA, 27 nt, Artificial Sequence
  Synthetic Polynucleotide
ugaaucuauuuuuuaaaaaucauccug

SEQ ID NO: 548 - RNA, 27 nt, Artificial Sequence
  Synthetic Polynucleotide
uugaaucuauuuuuuaaaaaucauccu

SEQ ID NO: 549 - RNA, 27 nt, Artificial Sequence
  Synthetic Polynucleotide
uuugaaucuauuuuuuaaaaaucaucc

SEQ ID NO: 550 - RNA, 27 nt, Artificial Sequence
  Synthetic Polynucleotide
auuugaaucuauuuuuuaaaaaucauc

SEQ ID NO: 551 - RNA, 27 nt, Artificial Sequence
  Synthetic Polynucleotide
cauuugaaucuauuuuuuaaaaaucau

SEQ ID NO: 552 - RNA, 27 nt, Artificial Sequence
  Synthetic Polynucleotide
acauuugaaucuauuuuuuaaaaauca

SEQ ID NO: 553 - RNA, 27 nt, Artificial Sequence
  Synthetic Polynucleotide
cacauuugaaucuauuuuuuaaaaauc

SEQ ID NO: 554 - RNA, 27 nt, Artificial Sequence
  Synthetic Polynucleotide
uaaacucgaguuauaggaagcguuuca

SEQ ID NO: 555 - RNA, 27 nt, Artificial Sequence
  Synthetic Polynucleotide
aacaauagcuuuuucuuccccuauaaa

SEQ ID NO: 556 - RNA, 27 nt, Artificial Sequence
  Synthetic Polynucleotide
aaacaauagcuuuuucuuccccuauaa

SEQ ID NO: 557 - RNA, 27 nt, Artificial Sequence
  Synthetic Polynucleotide
uguaaacaauagcuuuuucuuccccua

SEQ ID NO: 558 - RNA, 27 nt, Artificial Sequence
  Synthetic Polynucleotide
uuguaaacaauagcuuuuucuuccccu

SEQ ID NO: 559 - RNA, 27 nt, Artificial Sequence
  Synthetic Polynucleotide
auuguaaacaauagcuuuuucuucccc

SEQ ID NO: 560 - RNA, 27 nt, Artificial Sequence
  Synthetic Polynucleotide
aauuguaaacaauagcuuuuucuuccc

SEQ ID NO: 561 - RNA, 27 nt, Artificial Sequence
  Synthetic Polynucleotide
uaauuguaaacaauagcuuuuucuucc

SEQ ID NO: 562 - RNA, 27 nt, Artificial Sequence
  Synthetic Polynucleotide
auaauuguaaacaauagcuuuuucuuc

SEQ ID NO: 563 - RNA, 27 nt, Artificial Sequence
  Synthetic Polynucleotide
uauaauuguaaacaauagcuuuuucuu

SEQ ID NO: 564 - RNA, 27 nt, Artificial Sequence
  Synthetic Polynucleotide
auauaauuguaaacaauagcuuuuucu

SEQ ID NO: 565 - RNA, 27 nt, Artificial Sequence
  Synthetic Polynucleotide
gauauaauuguaaacaauagcuuuuuc

SEQ ID NO: 566 - RNA, 27 nt, Artificial Sequence
  Synthetic Polynucleotide
ugauauaauuguaaacaauagcuuuuu

SEQ ID NO: 567 - RNA, 27 nt, Artificial Sequence
  Synthetic Polynucleotide
gugauauaauuguaaacaauagcuuuu

SEQ ID NO: 568 - RNA, 27 nt, Artificial Sequence
  Synthetic Polynucleotide
ggugauauaauuguaaacaauagcuuu

SEQ ID NO: 569 - RNA, 27 nt, Artificial Sequence
  Synthetic Polynucleotide
uggugauauaauuguaaacaauagcuu

SEQ ID NO: 570 - RNA, 27 nt, Artificial Sequence
  Synthetic Polynucleotide
auggugauauaauuguaaacaauagcu

SEQ ID NO: 571 - RNA, 27 nt, Artificial Sequence
  Synthetic Polynucleotide
aauggugauauaauuguaaacaauagc

SEQ ID NO: 572 - RNA, 27 nt, Artificial Sequence
  Synthetic Polynucleotide
uaauggugauauaauuguaaacaauag

SEQ ID NO: 573 - RNA, 27 nt, Artificial Sequence
  Synthetic Polynucleotide
uuaauggugauauaauuguaaacaaua

SEQ ID NO: 574 - RNA, 27 nt, Artificial Sequence
  Synthetic Polynucleotide
cuuaauggugauauaauuguaaacaau

SEQ ID NO: 575 - RNA, 27 nt, Artificial Sequence
  Synthetic Polynucleotide
ccuuaauggugauauaauuguaaacaa

SEQ ID NO: 576 - RNA, 27 nt, Artificial Sequence
  Synthetic Polynucleotide
uugccuuaauggugauauaauuguaaa

SEQ ID NO: 577 - RNA, 27 nt, Artificial Sequence
  Synthetic Polynucleotide
gcaguugccuuaauggugauauaauug

SEQ ID NO: 578 - RNA, 27 nt, Artificial Sequence
  Synthetic Polynucleotide
agaauacaaagcaggguguagcaguug

SEQ ID NO: 579 - RNA, 27 nt, Artificial Sequence
  Synthetic Polynucleotide
cagaauacaaagcaggguguagcaguu

SEQ ID NO: 580 - RNA, 27 nt, Artificial Sequence
  Synthetic Polynucleotide
ccagaauacaaagcaggguguagcagu

SEQ ID NO: 581 - RNA, 36 nt, Artificial Sequence
  Synthetic Polynucleotide
uucauaaacaaugaauggcagcagccgaaaggcugc

SEQ ID NO: 582 - RNA, 36 nt, Artificial Sequence
  Synthetic Polynucleotide
ucauaaacaaugaauggcaagcagccgaaaggcugc

SEQ ID NO: 583 - RNA, 36 nt, Artificial Sequence
  Synthetic Polynucleotide
gaaacgugguugugaugaaggcagccgaaaggcugc

SEQ ID NO: 584 - RNA, 36 nt, Artificial Sequence
  Synthetic Polynucleotide
guugugaugaagguagcugagcagccgaaaggcugc

SEQ ID NO: 585 - RNA, 36 nt, Artificial Sequence
  Synthetic Polynucleotide
gguggaugaaacucaguuuagcagccgaaaggcugc

SEQ ID NO: 586 - RNA, 36 nt, Artificial Sequence
  Synthetic Polynucleotide
caguuuaagaagauccucgggcagccgaaaggcugc

SEQ ID NO: 587 - RNA, 36 nt, Artificial Sequence
  Synthetic Polynucleotide
uuuaagaagauccucggcuagcagccgaaaggcugc

SEQ ID NO: 588 - RNA, 36 nt, Artificial Sequence
  Synthetic Polynucleotide
guucuagaauuugaauugaugcagccgaaaggcugc

SEQ ID NO: 589 - RNA, 36 nt, Artificial Sequence
  Synthetic Polynucleotide
ccuuuuaaacgacaacaauagcagccgaaaggcugc

SEQ ID NO: 590 - RNA, 36 nt, Artificial Sequence
  Synthetic Polynucleotide
augauuuuuaaaaaauagaugcagccgaaaggcugc

SEQ ID NO: 591 - RNA, 22 nt, Artificial Sequence
  Synthetic Polynucleotide
ugccauucauuguuuaugaagg

SEQ ID NO: 592 - RNA, 22 nt, Artificial Sequence
  Synthetic Polynucleotide
uugccauucauuguuuaugagg

SEQ ID NO: 593 - RNA, 22 nt, Artificial Sequence
  Synthetic Polynucleotide
cuucaucacaaccacguuucgg

SEQ ID NO: 594 - RNA, 22 nt, Artificial Sequence
  Synthetic Polynucleotide
ucagcuaccuucaucacaacgg

SEQ ID NO: 595 - RNA, 22 nt, Artificial Sequence
  Synthetic Polynucleotide
uaaacugaguuucauccaccgg

SEQ ID NO: 596 - RNA, 22 nt, Artificial Sequence
  Synthetic Polynucleotide
ccgaggaucuucuuaaacuggg

SEQ ID NO: 597 - RNA, 22 nt, Artificial Sequence
  Synthetic Polynucleotide
uagccgaggaucuucuuaaagg

SEQ ID NO: 598 - RNA, 22 nt, Artificial Sequence
  Synthetic Polynucleotide
aucaauucaaauucuagaacgg

SEQ ID NO: 599 - RNA, 22 nt, Artificial Sequence
  Synthetic Polynucleotide
uauuguugucguuuaaaagggg

SEQ ID NO: 600 - RNA, 22 nt, Artificial Sequence
  Synthetic Polynucleotide
aucuauuuuuuaaaaaucaugg

SEQ ID NO: 601 - DNA, 93 bp, Homo sapiens
aaccagcagcccgaggtcttctgcaaccagattttcataaacaatgaatggcacgatgccgtcagcaggaaaacattccccaccgtcaatccg

SEQ ID NO: 602 - DNA, 101 bp, Homo sapiens
acctacctggcggccttggagaccctggacaatggcaagccctatgtcatctcctacctggtggatttggacatggtcctcaaatgtctccggtattatgc

SEQ ID NO: 603 - DNA, 51 bp, Homo sapiens
ccgtggaatttcccgctcctgatgcaagcatggaagctgggcccagccttg

SEQ ID NO: 604 - DNA, 59 bp, Homo sapiens
gccttggcaactggaaacgtggttgtgatgaaggtagctgagcagacacccctcaccgc

SEQ ID NO: 605 - DNA, 71 bp, Homo sapiens
gagcaggggccgcaggtggatgaaactcagtttaagaagatcctcggctacatcaacacggggaagcaaga

SEQ ID NO: 606 - DNA, 52 bp, Homo sapiens
tctcttgggtcaagaaagttctagaatttgaattgataaacatggtgggttg

SEQ ID NO: 607 - DNA, 93 bp, Homo sapiens
tgagggtaagagtatatgaggaaccttttaaacgacaacaatactgctagctttcaggatgatttttaaaaaatagattcaaatgtgttatcc

SEQ ID NO: 608 - DNA, 29 bp, Artificial Sequence
  Synthetic Polynucleotide
gaaacucaguuuagcagccgaaaggcugc

SEQ ID NO: 609 - DNA, 20 bp, Artificial Sequence
  Synthetic Polynucleotide
gguggaugaaacucaguuua

SEQ ID NO: 610 - DNA, 2076 bp, Homo sapiens
attggctgccgcgcggggcggggagcggggtcggctcagtggccctgagaccctagctctgctctcggtccgctcgctgtccgctagcccgctgcgatgttgcgcgctgccgcccgcttcgggccccgcctgggccgccgcctcttgtcagccgccgccacccaggccgtgcctgcccccaaccagcagcccgaggtcttctgcaaccagattttcataaacaatgaatggcacgatgccgtcagcaggaaaacattccccaccgtcaatccgtccactggagaggtcatctgtcaggtagctgaaggggacaaggaagatgtggacaaggcagtgaaggccgcccgggccgccttccagctgggctcaccttggcgccgcatggacgcatcacacaggggccggctgctgaaccgcctggccgatctgatcgagcgggaccggacctacctggcggccttggagaccctggacaatggcaagccctatgtcatctcctacctggtggatttggacatggtcctcaaatgtctccggtattatgccggctgggctgataagtaccacgggaaaaccatccccattgacggagacttcttcagctacacacgccatgaacctgtgggggtgtgcgggcagatcattccgtggaatttcccgctcctgatgcaagcatggaagctgggcccagccttggcaactggaaacgtggttgtgatgaaggtagctgagcagacacccctcaccgccctctatgtggccaacctgatcaaggaggctggctttccccctggtgtggtcaacattgtgcctggatttggccccacggctggggccgccattgcctcccatgaggatgtggacaaagtggcattcacaggctccactgagattggccgcgtaatccaggttgctgctgggagcagcaacctcaagagagtgaccttggagctgggggggaagagccccaacatcatcatgtcagatgccgatatggattgggccgtggaacaggcccacttcgccctgttcttcaaccagggccagtgctgctgtgccggctcccggaccttcgtgcaggaggacatctatgatgagtttgtggagcggagcgttgcccgggccaagtctcgggtggtcgggaacccctttgatagcaagaccgagcaggggccgcaggtggatgaaactcagtttaagaagatcctcggctacatcaacacggggaagcaagagggggcgaagctgctgtgtggtgggggcattgctgctgaccgtggttacttcatccagcccactgtgtttggagatgtgcaggatggcatgaccatcgccaaggaggagatcttcgggccagtgatgcagatcctgaagttcaagaccatagaggaggttgttgggagagccaacaattccacgtacgggctggccgcagctgtcttcacaaaggatttggacaaggccaattacctgtcccaggccctccaggcgggcactgtgtgggtcaactgctatgatgtgtttggagcccagtcaccctttggtggctacaagatgtcggggagtggccgggagttgggcgagtacgggctgcaggcatacactgaagtgaaaactgtcacagtcaaagtgcctcagaagaactcataagaatcatgcaagcttcctccctcagccattgatggaaagttcagcaagatcagcaacaaaaccaagaaaaatgatccttgcgtgctgaatatctgaaaagagaaatttttcctacaaaatctcttgggtcaagaaagttctagaatttgaattgataaacatggtgggttggctgagggtaagagtatatgaggaaccttttaaacgacaacaatactgctagctttcaggatgatttttaaaaaatagattcaaatgtgttatcctctctctgaaacgcttcctataactcgagtttataggggaagaaaaagctattgtttacaattatatcaccattaaggcaactgctacaccctgctttgtattctgggctaagattcattaaaaactagctgctcttaacttacaaaaaaaaaaaaaaaa

SEQ ID NO: 611 - DNA, 2124 bp, Macaca fascicularis
ccctgccctccgtccgctcgcagcccgctatcctgctgccatgttgcgtgctgccgcccgcttcgggccccgcctgggcctccgcttcttgtcagccgccgccacccaggccgtgcccgcccccaaccagcagcccgaggtcttctgcaacaaggacttcagctcccagaagccaggacttggctgttggacttccagtgctggagtggccagggctgtcagtgaaagcttatggaatgcgtgggtgaatgaatctcaacttttacaaaacaaaatcttcataaacaatgaatggcacaatgccgtcagcaggaaaacattccccacagtcaatccgtccactggagaggtcatctgccaggtagctgaaggggacaaggaagatgtggacaaggcagtgaaggccgcccgggccgccttccagctgggctcaccttggcgtcgcatggacgcgtcacacaggggccggctgctgaaccggctggctgatctgatcgagcgggaccggacctacctggcggccttggagactctggacaatggcaaaccctatgtcacctcctacctggtggatttggacatggtcctcaaatgtctccggtattatgccggctgggctgataagtaccacgggaaaaccattcccattgacggagacttcttcagctacacccgccatgaacctgtgggggtgtgcgggcagatcattccgtggaatttcccactcctgatgcaagcatggaagctgggcccagccttggcgactggaaacgtggttgtgatgaaggtagctgagcagacacccctcactgccctctatgtggccaacctgatcaaggaggccggctttccccctggtgtggtcaacattgttcctggatttggccccacagccggggccgccatcgcctcccatgaggatgtggacaaagtggcattcacaggctccaccgagattggccgcctcatccaggttgctgccgggagcagcaatctcaagagagtgaccttggagctggggggaaagagccccaacatcatcatgtcagatgccgacatggactgggccgtggagcaggcccacttcgccctgttcttcaaccagggccaatgctgctgtgctggctcccggaccttcgtgcaggaggacatctatgacgagtttgtggagcggagcgttgcccgggccaagtctcgggtggtcgggaacccctttgacagcaagaccgagcaggggccgcaggtggatgaaactcagtttaagaagatcctcggctacatcaacactgggaaacaagagggggcgaagctgctgtgtggtgggggcattgctgctgaccgtggttacttcatccagcccaccgtgtttggagatgtgcaggatggcatgaccatcgccaaggaggagatcttcgggccagtgatgcagatcctgaagttcaagaccatagaggaagttgttgggagagccaacaattccacgtacgggctggccgcagctgtcttcacaaaggatttggacaaggccaattacctgtcccaggccctccaggcgggcaccgtgtgggtcaactgctatgatgtgtttggagcccagtcaccctttggcggctacaagatgtcgggcagtggccgggagctgggcgagtacggcctgcaggcatacactgaagtgaaaactatcacagtcaaagtgcctcagaagaactcataagaaccatgtgggctttctccctcagccattgatggaaagttcagcaagatcagcgacaaaaccaagaaaaatgatccttgcgtgctgaatatctgaaaagagaaattcttcctacaaaatctcttgggtcaagaaagttctagaatttgaattgataaacatggtgggttggctgagggtaagagtctatgagaaaccttttaaatgacaacaatactgctagctttcagggtgcatttttaaaaaatagattcaaatgtcttatcctctctctgaaacgcctcctgtaacttgagtttataggggaagaaaaagccattgtttacaattatatcagcatcaaggcaactgctacaccctgctttgtattctgggctaagattcattaaaaacaagctgctctcaactta

SEQ ID NO: 612 - DNA, 3883 bp, Mus musculus
attctcttcgccgccatatctgcacagatgtgagccttaggcgccagccaccctgctaggagcgcacaccactctggctaggctttctcagggttctgcaaactccatctctgacttggctttgggagccaggggtcgcgccccttaggccgtgaggggctgggactccctgaccacgcccccgtgtctccgcctcccattggcggctgcagggggcggaggcgaggacttgttcttcaacgctgcagtcgccctccgatcggcaaggcttctctcggctccgttcggctcggctcgcccatttcagttcagttcgggtcagttaagctccgctcagttcagcatgctgcgcgccgcactcaccactgtccgccgcggaccgcgcctgagccgcctgttgtccgccgccgccaccagcgcggtgccagcccccaaccatcagcctgaggtcttctgcaaccagatcttcattaacaatgagtggcacgacgccgtcagcaggaaaacatttcccaccgtcaacccttccacaggggaggtcatctgccaggtggccgaagggaacaaggaggacgtagacaaggcagtgaaggctgctcgtgcagccttccagctgggctcgccctggcgccgcatggatgcatctgaccggggccggctgttgtaccgattggcggatctcattgaacgggaccggacctacctagcggccttggagaccctggacaacggcaagccttatgtcatctcgtacctggtggatttggacatggtcctgaaatgtctccgctattacgctggctgggctgacaagtaccatgggaaaaccattcccatcgacggcgacttcttcagctatacccgccatgagcctgtgggcgtgtgtggacagatcattccgtggaacttcccgctcctgatgcaagcatggaaactgggcccagccctggcaaccgggaacgtggtggtgatgaaggtggccgagcagacaccgctcaccgcgctctacgtggccaacttgatcaaggaggcaggctttccccctggcgtggtcaatatcgttcccggattcggccctaccgccggggctgccatcgcatcccatgagggtgtggacaaagtggcgttcacaggctccacggaggttggtcacctaatccaggtggccgccgggagcagcaacctcaagagagtaaccctggagctggggggaaagagtcccaacatcatcatgtccgacgctgacatggactgggctgtggagcaggcccactttgccctgttcttcaaccagggccagtgctgctgcgcaggctcccggaccttcgtgcaggagaatgtgtatgacgaattcgtggaacgcagcgtggctcgggccaagtctcgggtggtggggaaccccttcgacagccggacggagcaggggcctcaggtggatgaaactcagtttaagaagatcctcggctacatcaaatcgggacaacaagaaggggcgaagctgctgtgtggtgggggcgctgccgcggaccgtggctactttatccagcccaccgtgttcggggacgtaaaagacggcatgaccattgccaaggaggagatctttggaccagtgatgcaaatcctcaaattcaagaccatcgaggaggttgtggggcgggccaatgattctaagtatgggctggcagccgccgtcttcacaaaggacctggataaagccaattacctgtcccaagctctgcaggctggcactgtgtggatcaactgctacgatgtgtttggggcccagtctccatttgggggctataagatgtcagggagtggcagggagctgggcgagtatggcctgcaggcgtacacagaagtgaagacggttactgtcaaagtgccacagaagaactcgtaaagcggcatgcctgcttcctcagcccgcacccgaaaacccaacaagatatactgagaaaaaccgccacacacactgcgcctccaaagagaaaccccttcaccaaagtgtcttgggtcaagaaagaattttataaacagggcggggctggtgggggggaaagctcctgataaactgggtaggggatgaagctcaatgcagaccgatcacgcgtccagatgtgcaggatgctgccttcaacctgcagtccctaagcagcaaatgagcaataaaaatcagcagatcaaagccacggggtcagttctctaagacgtaaattctgagtcttatctctgttgcattccgtaactctctgctttgggaggagacaaggccgtccttagaattgaattagctctgtggaacactagcgccttggtgtttactggtcagaagtcatgaaaggcaggacccctctctattcctggatacacgggacgccaggatgtccccactatttggtgatatcatgtatatctcattaccctcatccccatcttggtacctgggatcttggttctcacaagtaattctaggctgacgaggagggatgtaagctaaagggaggggagtcactattcttggctgcagctacattttggaatttcacactggcctatctcacaggccagaggaggtagtgacacccgtttgatcctttccaagggtgagccaggttaggtttcaaccaagggacctcacggctcggcatcagttgcatgctgctaactaacctggagtcgattcagccaaaagacagtttccagaagtggctctgttctcccaatctgttctgcggcctctttgaggcacagagcagagcagagccatgtcatgtgcacagtgcaaggtctgcctctcgaactaccagaaccaagagagattggaggtattagtgacagtggcctgatggaggtggcaggtgatggcccgtgaaggtagattttcagggaataagagaatgtggcagtgactggcagctgcaatgcagactctggttcaggctatggtcactgcagatgcctggatgtgtgacagatgtgatgacattgctgaaagacagagggacccactcccagtggaaggccaagagagctcttgagcagggctccagttacgtgagctttctccctcttgtaccagaaggttccattcctagaggttttagctacctgtgacctctgccctgtgtataaatggggttactgtgactatcccaaccctgccctgtcctaaagtaccttcaagggccaccttgtgcccaacctgtagattattccctatacagaaatgctcatgatgtttggcataaaaaaaaaattaagccagcaattagcaatagtcattgtgagaagttacacaactcttggtgccgctacgcttgtgttttggggtgttgagttagctctgcatgggtgctgcaatactcaatcctggaagcccaggagccaagatggttgctcgtagctgataggtgggcaaatatgccacgtgggtacaacggctgcaggcatgatgctcacatcgggcacagggccgcaccgatgcatgcagcctggaccagtgtgtgcttattttcagaatttccgtttatactcttatgctatgcatgacgattagctgcctggtacctgccctgagcagggataagcaggtcaagtccaaggtccacaggagctgggaagcttaggggcgaaggtcacagatcttactcacctagtgagtgaacaaggcgtggagagcaagctgccatcacaggcacaagaaacggacggtgagcttagctttagaactagccagtcagaggcagagctgagggtagaaggctgatgaagccctgaagttgtccttcgacctccatatacacatccctgtatgtgcatgcgcactcaatgaaataaataagtaaatacaatttttaaagatcaaaaaaaaaaaaaaaaa